SEQUENCE LISTING (57 sequences)

SEQ ID NO: 1 - DNA, 774 bp, Human
agggcaagttaagggaatagtggaatgaaggttcatttttcattctcacaaactaatgaaaccctgcttatcttaaaccaacctgctcactggagcagggaggacaggaccagcataaaaggcagggcagagtcgactgttgcttacactttcttctgacataacagtgttcactagcaacctcaaacagacaccatggtgcatctgactcctgaggagaagactgctgtcaatgccctgtggggcaaagtgaacgtggatgcagttggtggtgaggccctgggcagattactggtggtctacccttggacccagaggttctttgagtcctttggggatctgtcctctcctgatgctgttatgggcaaccctaaggtgaaggctcatggcaagaaggtgctaggtgcctttagtgatggcctggctcacctggacaacctcaagggcactttttctcagctgagtgagctgcactgtgacaagctgcacgtggatcctgagaacttcaggctcttgggcaatgtgctggtgtgtgtgctggcccgcaactttggcaaggaattcaccccacaaatgcaggctgcctatcagaaggtggtggctggtgtggctaatgccctggctcacaagtaccattgagatcctggactgtttcctgataaccataagaagaccctatttccctagattctattttctgaacttgggaacacaatgcctacttcaagggtatggcttctgcctaataaagaatgttcagctcaacttcctgat

SEQ ID NO: 2 - DNA, 4953 bp, Human
gaacgagtgggaacgtagctggtcgcagagggcaccagcggctgcaggacttcaccaagggaccctgaggctcgtgagcagggacccgcggtgcgggttatgctgggggctcagatcaccgtagacaactggacactcaggaccacgccatggaggagctgcaggatgattatgaagacatgatggaggagaatctggagcaggaggaatatgaagacccagacatccccgagtcccagatggaggagccggcagctcacgacaccgaggcaacagccacagactaccacaccacatcacacccgggtacccacaaggtctatgtggagctgcaggagctggtgatggacgaaaagaaccaggagctgagatggatggaggcggcgcgctgggtgcaactggaggagaacctgggggagaatggggcctggggccgcccgcacctctctcacctcaccttctggagcctcctagagctgcgtagagtcttcaccaagggtactgtcctcctagacctgcaagagacctccctggctggagtggccaaccaactgctagacaggtttatctttgaagaccagatccggcctcaggaccgagaggagctgctccgggccctgctgcttaaacacagccacgctggagagctggaggccctggggggtgtgaagcctgcagtcctgacacgctctggggatccttcacagcctctgctcccccaacactcctcactggagacacagctcttctgtgagcagggagatgggggcacagaagggcactcaccatctggaattctggaaaagattcccccggattcagaggccacgttggtgctagtgggccgcgccgacttcctggagcagccggtgctgggcttcgtgaggctgcaggaggcagcggagctggaggcggtggagctgccggtgcctatacgcttcctctttgtgttgctgggacctgaggccccccacatcgattacacccagcttggccgggctgctgccaccctcatgtcagagagggtgttccgcatagatgcctacatggctcagagccgaggggagctgctgcactccctagagggcttcctggactgcagcctagtgctgcctcccaccgatgccccctccgagcaggcactgctcagtctggtgcctgtgcagagggagctacttcgaaggcgctatcagtccagccctgccaagccagactccagcttctacaagggcctagacttaaatgggggcccagatgaccctctgcagcagacaggccagctcttcgggggcctggtgcgtgatatccggcgccgctacccctattacctgagtgacatcacagatgcattcagcccccaggtcctggctgccgtcatcttcatctactttgctgcactgtcacccgccatcaccttcggcggcctcctgggagaaaagacccggaaccagatgggagtgtcggagctgctgatctccactgcagtgcagggcattctcttcgccctgctgggggctcagcccctgcttgtggtcggcttctcaggacccctgctggtgtttgaggaagccttcttctcgttctgcgagaccaacggtctagagtacatcgtgggccgcgtgtggatcggcttctggctcatcctgctggtggtgttggtggtggccttcgagggtagcttcctggtccgcttcatctcccgctatacccaggagatcttctccttcctcatttccctcatcttcatctatgagactttctccaagctgatcaagatcttccaggaccacccactacagaagacttataactacaacgtgttgatggtgcccaaacctcagggccccctgcccaacacagccctcctctcccttgtgctcatggccggtaccttcttctttgccatgatgctgcgcaagttcaagaacagctcctatttccctggcaagctgcgtcgggtcatcggggacttcggggtccccatctccatcctgatcatggtcctggtggatttcttcattcaggatacctacacccagaaactctcggtgcctgatggcttcaaggtgtccaactcctcagcccggggctgggtcatccacccactgggcttgcgttccgagtttcccatctggatgatgtttgcctccgccctgcctgctctgctggtcttcatcctcatattcctggagtctcagatcaccacgctgattgtcagcaaacctgagcgcaagatggtcaagggctccggcttccacctggacctgctgctggtagtaggcatgggtggggtggccgccctctttgggatgccctggctcagtgccaccaccgtgcgttccgtcacccatgccaacgccctcactgtcatgggcaaagccagcaccccaggggctgcagcccagatccaggaggtcaaagagcagcggatcagtggactcctggtcgctgtgcttgtgggcctgtccatcctcatggagcccatcctgtcccgcatccccctggctgtactgtttggcatcttcctctacatgggggtcacgtcgctcagcggcatccagctctttgaccgcatcttgcttctgttcaagccacccaagtatcacccagatgtgccctacgtcaagcgggtgaagacctggcgcatgcacttattcacgggcatccagatcatctgcctggcagtgctgtgggtggtgaagtccacgccggcctccctggccctgcccttcgtcctcatcctcactgtgccgctgcggcgcgtcctgctgccgctcatcttcaggaacgtggagcttcagtgtctggatgctgatgatgccaaggcaacctttgatgaggaggaaggtcgggatgaatacgacgaagtggccatgcctgtgtgaggggcgggcccaggccctagaccctcccccaccattccacatccccaccttccaaggaaaagcagaagttcatgggcacctcatggactccaggatcctcctggagcagcagctgaggccccagggctgtgggtggggaaggaaggcgtgtccaggagaccttccacaaagggtagcctggcttttctggctggggatggccgatggggcccacattagggggtttgttgcacagtccctcctgttgccacactttcactggggatcccgtgctggaagacttagatctgagccctccctcttcccagcacaggcaggggtagaagcaaaggcaggaggtgggtgagcgggtggggtgcttgctgtgtgaccttgggcaagtcccttgacctttccagcctatatttcctcttctgtaaaatgggtatattgatgataatacccacattacaggatggttactgaggaccaaagatacatgtaaaatagggctttgtaaactccacagggactgttctatagcagtcatcatttgtctttgaacgtacccaaggtcacatagctgggatttgaactgagccgtgcagctgggatttgaaccaggccttctgatttcaaggtccgagctctgtcctctgtcagtcatgcgtccactttcccttcccctgtgactcctcccttccccactctgctcccagcccctaccttgagaccctcttctctgggcccagagagaggcgtcctggtgaggacaaggtacaggcaaggatgatccagggattgggcctgggactcaggcctcctaagtgtttggttcctccctccaaacactcattagttcactcattcattcattccacaaacatttactgagggccccggaatcagtggactccgaggggactgagacaagccctgccctggggtgggggtggggggcaaggtacagttgattctacatttggatagggagtgggggagggtgggaaggtaggggcgggagagtgagggggtttgtaatttattaattgcgtattttctaagagttttcaacatagtttggcttcacacacaacttcaggcccctcatttgagagccattatcctcaactccatctaaactgaatcttggggagaacccagatctgaccaattggggtaggagacagcaggctctccaagaacatgggcaaatttatttttttataaaacaaaaagataaaaagagttgaaagacgtgaaagtggtgagagatggaggaaacagaatcaggaagtggtagaaaagagaggaggtggctgggcgcagtggctcacgtttgtaatcccagcactttgggaggccaagttgggcggatcatttgaggtcaggagtttgagaccagcctggccaacatggtgaaaccccgttactactaaaaatacaaaaattagctgggtgtctcgtggcaggcacctgtaatcccagctacttagaaggctgaggcaagagaatcacctgaacccaggaggtggaggttgcagtgagccaagattgcaccactgcactccagcctgggcaacagagcgagaccctgtctcaaaaaaaaaaaaaaaaaaaaaaaaaaaacggaaggaaacatcagccttgggggccacagactcaacatgtgtgtgtggtggggttccagcccaacatagagtaacattatttgtacctcccaggctagctcagtccatgggaggctctcctgtccctgaaagctgacacccacctttcaccacttcgcccatgctacagttcagtttcctcgtctgtaaaatggggatgataatggtacctaccttgcagtgttgttataaggattaaaggagacagtgcaagaaaaggccttggttggtgaagagcccaacctcggaggggagctgctgggatcctccttatcttgactgggatgtccctgtctccccctccccttgctccttgaacatggccaaggaaagtgaaaaacaaaaattattcactctgctagcacccttccccttgatgcctgggaataggttttgccaataaacgtatctgtgttgga

SEQ ID NO: 3 - DNA, 2666 bp, Human
aaaatgcctcccctgcctatcagctgatgatggccgcaggaaggtgggcctggaagataacagctagcaggctaaggtcagacactgacacttgcagttgtctttggtagtttttttgcactaacttcaggaaccagctcatgatctcaggatgtatggaaaaataatctttgtattactattgtcagaaattgtgagcatatcagcattaagtaccactgaggtggcaatgcacacttcaacttcttcttcagtcacaaagagttacatctcatcacagacaaatgatacgcacaaacgggacacatatgcagccactcctagagctcatgaagtttcagaaatttctgttagaactgtttaccctccagaagaggaaaccgagataacactcattatttttggggtgatggctggtgttattggaacgatcctcttaatttcttacggtattcgccgactgataaagaaaagcccatctgatgtaaaacctctcccctcacctgacacagacgtgcctttaagttctgttgaaatagaaaatccagagacaagtgatcaatgagaatctgttcaccaaaccaaatgtggaaagaacacaaagaagacataagacttcagtcaagtgaaaaattaacatgtggactggacactccaataaattatatacctgcctaagttgtacaatttcagaatgcaattttcattataatgagttccagtgactcaatgatggggaaaaaaatctctgctcattaatatttcaagataaagaacaaatgtttccttgaatgcttgcttttgtgtgttagcataatttttagaattgtttgagaattctgatccaaaactttagttgaattcatctacgtttgtttaatattaacttaacctattctattgtattataatgatgattctgtcaaatgaaaggcttgaaatacctagatgaagtttagattttcttcctattgtaaacttttgagtctggtttcattgttttaaataaattaaggggacactaaagtcctatcattcatttccttcattgctgaacaggcaagatataatattacatgaatgattactatattttgttcacactaataaagcttatgctcagaaatgccatacacacacacacacacacacacaaacacacacatttatcatttaatgcataaatcaacacaaaaggttttcccattaatatgaaatattacatatatataagtgccatatttaaaataatttgtctaacagtagaactgtgtcggagcactcactgaagcttgcattccactgaaagagttatttgtgtaagtagagtatccggagaaggaaaagaacttacgacctttctttataacagaaactcaactctaaattcaacaagatgtgcaaaccggacatgcaggtgaatattttaataggttactataaggttctcaattaaattctttaatctgtccagtcccagtttctcttattaataaaactttggaaattgctttaaaccatttaaaggaaatttctagatatagaaactaaggactgtgactatacagctgtcactcatttgtagtaaaacttaaaaagcaaaaacaaaaaacaaaaaagaccttcctgtgatactttatttccgaactaataaaaatctatatgactttttattattgtgtgataaccaagtaaatgttttctattttgcatattttcaggcatggtaacagaaatttaccttttaataaattaaaaaatctaaattttaacctacttgtatgttcggagagtgtttttgtactatattgactacttaaaatagagaatgagactaagaagggaacatttctgttgatacatgttttttaaaagtaattttaagagcattattaggttaattaatccaattaatgacccaaatgccaaggtaattttaaatttacatttttaataaaagcaacatgttgaaacaagagagggtgagattaacctttttgctaaagtaatttacaagtcaaagacaggaagagatcagagtgaatgtgccttcttaaccagagctacagaatttagtgaataattaaagtacaaactgctttgacctccttgaacttttccaagcaatttctctgtacttctatatatgaatgtcttagccaattttctgctactataacagaatacgacagactgggtaatttaaaaagaaaagaaatttattttcttcctagttctggaggctgggaaggcgaagggcatggcactgacatctgccttgtaactgatgagaaccttcttactgcatgataacaaagcagcaaggcaagcaaaagcgtaagatgaagagagaggaaatgaagccaaacacatcctttcatcagaagcccattccctctataaggcgttattacatttatgagaatggagtcctcatgacctaatcgtgaccttaaaggcccctcccaacactgttacaatggcaattaaatttcaacaaaggttccagaggtgacattcgaatcagcaatgaaattttcatagttaaatttggtattcgtgggggaagaaatgaccatttcccttgtatttttataattaaatcagcaaaatattgtaataaagaaatctttcctgtgaagataccatgaccccaaaaaaaaaaa

SEQ ID NO: 4 - DNA, 573 bp, Human
ctccattccattatacctttgagtatataaaacagctacaatattccagggccagtcacttgccatttctcataacagcgtcagagagaaagaactgactgaaacgtttgagatgaagaaagttctcctcctgatcacagccatcttggcagtggctgttggtttcccagtctctcaagaccaggaacgagaaaaaagaagtatcagtgacagcgatgaattagcttcagggttttttgtgttcccttacccatatccatttcgcccacttccaccaattccatttccaagatttccatggtttagacgtaattttcctattccaatacctgaatctgcccctacaactccccttcctagcgaaaagtaaacaagaaggaaaagtcacgataaacctggtcacctgaaattgaaattgagccacttccttgaagaatcaaaattcctgttaataaaagaaaaacaaatgtaattgaaatagcacacagcattctctagtcaatatctttagtgatcttctttaataaacttgaaagcaaagattttggtttcttaatttccacaaaaaaaaaa

SEQ ID NO: 5 - DNA, 601 bp, Human
gggagatttcaacgtgtttaaatacatcagccatctaggaaaggacatctcttgagacttcacttcagcttcactgacttctggattctcctcttgagtaaaaggactcagccaactatgaagttttttgtttttgctttaatcttggctctcatgctttccatgactggagctgattcacatgcaaagagacatcatgggtataaaagaaaattccatgaaaagcatcattcacatcgaggctatagatcaaattatctgtatgacaattgatatcttcagtaatcacggggcatgattatggaggtttgactggcaaattcgctttggactcgtgtattctcatttgtcataccgcatcacactaccactgctttttgaagaattatcataaggcaatgcagaataaaagaaataccatgatttagtgaattctgtgtttcaggatacttcccttcctaattatcatttgattagatacttgcaatttaaatgttaagctgttttcactgctgtttctgagtaatagaaattcattcctctccaaaagcaataaaattcaagcacattattatgtgaaaaaaaaaaaaaaaaaaaaaaa

SEQ ID NO: 6 - DNA, 578 bp, Human
gagtgtttaaatacattggccctctagggtagcacatcatctcttgaagcttcacttcaacttcactacttctgtagtctcatcttgagtaaaagagaacccagccaactatgaagttccttgtctttgccttcatcttggctctcatggtttccatgattggagctgattcatctgaagagtatgggtatggcccttatcagccagttccagaacaaccactatacccacaaccataccaaccacaataccaacaatataccttttaatatcatcagtaactgcaggacatgattattgaggcttgattggcaaatacgacttctacatccatattctcatctttcataccatatcacactactaccactttttgaagaatcatcaaagagcaatgcaaatgaaaaacactataatttactgtatactctttgtttcaggatacttgccttttcaattgtcacttgatgatataattgcaatttaaactgttaagctgtgttcagtactgtttctgaataatagaaatcacttctctaaaagcaataaatttcaagcacatttttacataaaaaaaa

SEQ ID NO: 7 - DNA, 426 bp, Human
gactcacagcccacagagttccacctgctcacaggttggctggctcagccaaggtggtgccctgctctgagcattcaggccaagcccatcctgcaccatggccaggtacagatgctgtcgcagccagagccggagcagatattaccgccagagacaaagaagtcgcagacgaaggaggcggagctgccagacacggaggagagccatgaggtgctgccgccccaggtacagaccgcgatgtagaagacactaattgcacaaaatagcacatccaccaaactcctgcctgagaatgttaccagacttcaagatcctcttgccacatcttgaaaatgccaccatccaataaaaatcaggagcctgctaaggaacaatgccgcctgtcaataaatgttgaaaagtcatcccaaaaaaaaaaaaaaaaaa

SEQ ID NO: 8 - DNA, 415 bp, Human
gcccctcattttggcagaacttaccatgtcgaccagccgcaaattaaagagtcatggcatgaggaggagcaagagccgatctcctcacaagggagtcaagagaggtggcagcaaaagaaaataccgtaagggcaacctgaaaagtaggaaacggggcgatgacgccaatcgcaattaccgctcccacttgtgagcccccagcgggctctgccctggtgcgcttcacacagcaccaagcagcaacaagaacagcagaaggggaactgccaaggagacctgatgttagatcaaagccagagaggagcctatggaatgtggatcaaatgccagttgtgacgaaatgaggaatgtatatgttggctgtttttccccaacatctcaataaaactttgaaagcagaaaaaaaaaaaaaaaa

SEQ ID NO: 9 - DNA, 727 bp, Human
agaccagaccaacagtaacaccaagggcaggtgggcaggcctccgccctcctcccctactccagggcccactgcagcctcagcccaggagccaccagatctcccaacaccatggtccgataccgcgtgaggagcctgagcgaacgctcgcacgaggtgtacaggcagcagttgcatgggcaagagcaaggacaccacggccaagaggagcaagggctgagcccggagcacgtcgaggtctacgagaggacccatggccagtctcactataggcgcagacactgctctcgaaggaggctgcaccggatccacaggcggcagcatcgctcctgcagaaggcgcaaaagacgctcctgcaggcaccggaggaggcatcgcagagagtccctaggtgaccccctcaaccagaactttctttcccaaaaggctgcagaaccaggaagagaacatgcagaaggcactaagcttcctgggcccctcacccccagctggaaattaagaaaaagtcgcccgaaacaccaagtgaggccatagcaattcccctacatcaaatgctcaagcccccagctggaagttaagagaaagtcacctgcccaagaaacaccgagtgaggccatagcaactcccctacatcaaatgctcaagccctgagttgccgccgagaagcccacaagatctgagtgaaatgagcaaaagtcacctgcccaataaagcttgacaagacactc

SEQ ID NO: 10 - DNA, 2892 bp, Human
agccccaaactcaccacctggccgtggacacctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtggggtgagtcatccctactcccaacatctggaggggaaagggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtcccacccctacctctagtaaatttaagtccacctcacgttctggcatcacttggcctttctggatgctggacacctgaagcttggaactcacctggccgaagctcgagcctcctgagtcctactgacctgtgctttctggtgtggagtccagggctgctaggaaaaggaatgggcagacacaggtgtatgccaatgtttctgaaatgggtataatttcgtcctctccttcggaacactggctgtctctgaagacttctcgctcagtttcagtgaggacacacacaaagacgtgggtgaccatgttgtttgtggggtgcagagatgggaggggtggggcccaccctggaagagtggacagtgacacaaggtggacactctctacagatcactgaggataagctggagccacaatgcatgaggcacacacacagcaaggatgacgctgtaaacatagcccacgctgtcctgggggcactgggaagcctagataaggccgtgagcagaaagaaggggaggatcctcctatgttgttgaaggagggactagggggagaaactgaaagctgattaattacaggaggtttgttcaggtcccccaaaccaccgtcagatttgatgatttcctagcaggacttacagaaataaagagctatcatgctgtggtttattatggtttgttacattgataggatacatactgaaatcagcaaacaaaacagatgtatagattagagtgtggagaaaacagaggaaaacttgcagttacgaagactggcaacttggctttactaagttttcagactggcaggaagtcaaacctattaggctgaggaccttgtggagtgtagctgatccagctgatagaggaactagccaggtgggggcctttccctttggatggggggcatatctgacagttattctctccaagtggagacttacggacagcatataattctccctgcaaggatgtatgataatatgtacaaagtaattccaactgaggaagctcacctgatccttagtgtccagggtttttactgggggtctgtaggacgagtatggagtacttgaataattgacctgaagtcctcagacctgaggttccctagagttcaaacagatacagcatggtccagagtcccagatgtacaaaaacagggattcatcacaaatcccatctttagcatgaagggtctggcatggcccaaggccccaagtatatcaaggcacttgggcagaacatgccaaggaatcaaatgtcatctcccaggagttattcaagggtgagccctttacttgggatgtacaggctttgagcagtgcagggctgctgagtcaaccttttattgtacaggggatgagggaaagggagaggatgaggaagcccccctggggatttggtttggtcttgtgatcaggtggtctatggggctatccctacaaagaagaatccagaaataggggcacattgaggaatgatactgagcccaaagagcattcaatcattgttttatttgccttcttttcacaccattggtgagggagggattaccaccctggggttatgaagatggttgaacaccccacacatagcaccggagatatgagatcaacagtttcttagccatagagattcacagcccagagcaggaggacgctgcacaccatgcaggatgacatgggggatgcgctcgggattggtgtgaagaagcaaggactgttagaggcaggctttatagtaacaagacggtggggcaaactctgatttccgtgggggaatgtcatggtcttgctttactaagttttgagactggcaggtagtgaaactcattaggctgagaaccttgtggaatgcagctgacccagctgatagaggaagtagccaggtgggagcctttcccagtgggtgtgggacatatctggcaagattttgtggcactcctggttacagatactggggcagcaaataaaactgaatcttgttttcagaccttaaaaaaaaaaaaaaaaaaaaa

SEQ ID NO: 11 - DNA, 503 bp, Human
gtacctgtctataaggagtcctgcttatcacaatgaatgttctcctgggcagcgttgtgatctttgccaccttcgtgactttatgcaatgcatcatgctatttcatacctaatgagggagttccaggagattcaaccaggaaatgcatggatctcaaaggaaacaaacacccaataaactcggagtggcagactgacaactgtgagacatgcacttgctacgaaacagaaatttcatgttgcacccttgtttctacacctgtgggttatgacaaagacaactgccaaagaatcttcaagaaggaggactgcaagtatatcgtggtggagaagaaggacccaaaaaagacctgttctgtcagtgaatggataatctaatgtgcttctagtaggcacagggctcccaggccaggcctcattctcctctggcctctaatagtcaatgattgtgtagccatgcctatcagtaaaaagatttttgagcaaacacttgaaaaaaaaaaa

SEQ ID NO: 12 - DNA, 3027 bp, Human
ggaccgactgtgtggaagcaccaggcatcagagatagagtcttccctggcattgcaggagagaatctgaagggatgatggatgcatcaaaagagctgcaagttctccacattgacttcttgaatcaggacaacgccgtttctcaccacacatgggagttccaaacgagcagtcctgtgttccggcgaggacaggtgtttcacctgcggctggtgctgaaccagcccctacaatcctaccaccaactgaaactggaattcagcacagggccgaatcctagcatcgccaaacacaccctggtggtgctcgacccgaggacgccctcagaccactacaactggcaggcaacccttcaaaatgagtctggcaaagaggtcacagtggctgtcaccagttcccccaatgccatcctgggcaagtaccaactaaacgtgaaaactggaaaccacatccttaagtctgaagaaaacatcctataccttctcttcaacccatggtgtaaagaggacatggttttcatgcctgatgaggacgagcgcaaagagtacatcctcaatgacacgggctgccattacgtgggggctgccagaagtatcaaatgcaaaccctggaactttggtcagtttgagaaaaatgtcctggactgctgcatttccctgctgactgagagctccctcaagcccacagataggagggaccccgtgctggtgtgcagggccatgtgtgctatgatgagctttgagaaaggccagggcgtgctcattgggaattggactggggactacgaaggtggcacagccccatacaagtggacaggcagtgccccgatcctgcagcagtactacaacacgaagcaggctgtgtgctttggccagtgctgggtgtttgctgggatcctgactacagtgctgagagcgttgggcatcccagcacgcagtgtgacaggcttcgattcagctcacgacacagaaaggaacctcacggtggacacctatgtgaatgagaatggcgagaaaatcaccagtatgacccacgactctgtctggaatttccatgtgtggacggatgcctggatgaagcgaccggatctgcccaagggctacgacggctggcaggctgtggacgcaacgccgcaggagcgaagccagggtgtcttctgctgtgggccatcaccactgaccgccatccgcaaaggtgacatctttattgtctatgacaccagattcgtcttctcagaagtgaatggtgacaggctcatctggttggtgaagatggtgaatgggcaggaggagttacacgtaatttcaatggagaccacaagcatcgggaaaaacatcagcaccaaggcagtgggccaagacaggcggagagatatcacctatgagtacaagtatccagaaggctcctctgaggagaggcaggtcatggatcatgccttcctccttctcagttctgagagggagcacagacgacctgtaaaagagaactttcttcacatgtcggtacaatcagatgatgtgctgctgggaaactctgttaatttcaccgtgattcttaaaaggaagaccgctgccctacagaatgtcaacatcttgggctcctttgaactacagttgtacactggcaagaagatggcaaaactgtgtgacctcaataagacctcgcagatccaaggtcaagtatcagaagtgactctgaccttggactccaagacctacatcaacagcctggctatattagatgatgagccagttatcagaggtttcatcattgcggaaattgtggagtctaaggaaatcatggcctctgaagtattcacgtctttccagtaccctgagttctctatagagttgcctaacacaggcagaattggccagctacttgtctgcaattgtatcttcaagaataccctggccatccctttgactgacgtcaagttctctttggaaagcctgggcatctcctcactacagacctctgaccatgggacggtgcagcctggtgagaccatccaatcccaaataaaatgcaccccaataaaaactggacccaagaaatttatcgtcaagttaagttccaaacaagtgaaagagattaatgctcagaagattgttctcatcaccaagtagccttgtctgatgctgtggagccttagttgagatttcagcatttcctaccttgtgcttagctttcagattatggatgattaaatttgatgacttatatgagggcagattcaagagccagcaggtcaaaaaggccaacacaaccataagcagccagacccacaaggccaggtcctgtgctatcacagggtcacctcttttacagttagaaacaccagccgaggccacagaatcccatccctttcctgagtcatggcctcaaaaatcagggccaccattgtctcaattcaaatccatagatttcgaagccacagagtctctccctggagcagcagactatgggcagcccagtgctgccacctgctgacgacccttgagaagctgccatatcttcaggccatgggttcaccagccctgaaggcacctgtcaactggagtgctctctcagcactgggatgggcctgatagaagtgcattctcctcctattgcctccattctcctctctctatccctgaaatccaggaagtccctctcctggtgctccaagcagtttgaagcccaatctgcaaggacatttctcaagggccatgtggttttgcagacaaccctgtcctcaggcctgaactcaccatagagacccatgtcagcaaacggtgaccagcaaatcctcttcccttattctaaagctgccccttgggagactccagggagaaggcattgcttcctccctggtgtgaactctttctttggtattccatccactatcctggcaactcaaggctgcttctgttaactgaagcctgctccttcttgttctgccctccagagatttgctcaaatgatcaataagctttaaattaaactctacttcaaaaaaaaaaaaaaaaaaaaaaaaaaaaaa

SEQ ID NO: 13 - DNA, 1777 bp, Human
agaagcccagtagacaaagaaggtaagggcagtgagaatgatgcatcttgcattccttgtgctgttgtgtctgccagtctgctctgcctatcctctgagtggggcagcaaaagaggaggactccaacaaggatcttgcccagcaatacctagaaaagtactacaacctcgaaaaggatgtgaaacagtttagaagaaaggacagtaatctcattgttaaaaaaatccaaggaatgcagaagttccttgggttggaggtgacagggaagctagacactgacactctggaggtgatgcgcaagcccaggtgtggagttcctgacgttggtcacttcagctcctttcctggcatgccgaagtggaggaaaacccaccttacatacaggattgtgaattatacaccagatttgccaagagatgctgttgattctgccattgagaaagctctgaaagtctgggaagaggtgactccactcacattctccaggctgtatgaaggagaggctgatataatgatctcttttgcagttaaagaacatggagacttttactcttttgatggcccaggacacagtttggctcatgcctacccacctggacctgggctttatggagatattcactttgatgatgatgaaaaatggacagaagatgcatcaggcaccaatttattcctcgttgctgctcatgaacttggccactccctggggctctttcactcagccaacactgaagctttgatgtacccactctacaactcattcacagagctcgcccagttccgcctttcgcaagatgatgtgaatggcattcagtctctctacggacctccccctgcctctactgaggaacccctggtgcccacaaaatctgttccttcgggatctgagatgccagccaagtgtgatcctgctttgtccttcgatgccatcagcactctgaggggagaatatctgttctttaaagacagatatttttggcgaagatcccactggaaccctgaacctgaatttcatttgatttctgcattttggccctctcttccatcatatttggatgctgcatatgaagttaacagcagggacaccgtttttatttttaaaggaaatgagttctgggccatcagaggaaatgaggtacaagcaggttatccaagaggcatccataccctgggttttcctccaaccataaggaaaattgatgcagctgtttctgacaaggaaaagaagaaaacatacttctttgcagcggacaaatactggagatttgatgaaaatagccagtccatggagcaaggcttccctagactaatagctgatgactttccaggagttgagcctaaggttgatgctgtattacaggcatttggatttttctacttcttcagtggatcatcacagtttgagtttgaccccaatgccaggatggtgacacacatattaaagagtaacagctggttacattgctaggcgagatagggggaagacagatatgggtgtttttaataaatctaataattattcatctaatgtattatgagccaaaatggttaatttttcctgcatgttctgtgactgaagaagatgagccttgcagatatctgcatgtgtcatgaagaatgtttctggaattcttcacttgcttttgaattgcactgaacagaattaagaaatactcatgtgcaataggtgagagaatgtattttcatagatgtgttattacttcctcaataaaaagttttattttgggcctgttccttaaaaaaaaaaaaaaaaaaa

SEQ ID NO: 14 - DNA, 3897 bp, Human
cagtttgcaaaagccagaggtgcaagaagcagcgactgcagcagcagcagcagcagcggcggtggcagcagcagcagcagcggcggcagcagcagcagcagcggaggcaccggtggcagcagcagcatcaccagcaacaacaacaaaaaaaaatcctcatcaaatcctcacctaagctttcagtgtatccagatccacatcttcactcaagccaggagagggaaagaggaaaggggggcaggaaaaaaaaaaaacccaacaacttagcggaaacttctcagagaatgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataaccagggagaggttattcacaacctcaccaaactagtatcattttaggggtgttgacacaccagttttgagtgtactgtgcctggtttgatttttttaaagtagttcctattttctatcccccttaaagaaaattgcatgaaactaggcttctgtaatcaatatcccaacattctgcaatggcagcattcccaccaacaaaatccatgtgaccattctgcctctcctcaggagaaagtaccctcttttaccaacttcctctgccatgtttttcccctgctcccctgagaccacccccaaacacaaaacattcatgtaactctccagccattgtaatttgaagatgtggatccctttagaacggttgccccagtagagttagctgataaggaaactttatttaaatgcatgtcttaaatgctcataaagatgttaaatggaattcgtgttatgaatctgtgctggccatggacgaatatgaatgtcacatttgaattcttgatctctaatgagctagtgtcttatggtcttgatcctccaatgtctaattttctttccgacacatttaccaaattgcttgagcctggctgtccaaccagactttgagcctgcatcttcttgcatctaatgaaaaacaaaaagctaacatctttacgtactgtaactgctcagagctttaaaagtatctttaacaattgtcttaaaaccagagaatcttaaggtctaactgtggaatataaatagctgaaaactaatgtactgtacataaattccagaggactctgcttaaacaaagcagtatataataactttattgcatatagatttagttttgtaacttagctttatttttcttttcctgggaatggaataactatctcacttccagatatccacataaatgctccttgtggccttttttataactaagggggtagaagtagttttaattcaacatcaaaacttaagatgggcctgtatgagacaggaaaaaccaacaggtttatctgaaggaccccaggtaagatgttaatctcccagcccacctcaacccagaggctactcttgacttagacctatactgaaagatctctgtcacatccaactggaaattccaggaaccaaaaagagcatccctatgggcttggaccacttacagtgtgataaggcctactatacattaggaagtggcagttctttactcgtcccctttcatcggtgcctggtactctggcaaatgatgatggggtgggagactttccattaaatcaatcaggaatgagtcaatcagcctttaggtctttagtccgggggacttggggctgagagagtataaataaccctgggctgtccagccttaatagacttctcttacattttcgtcctgtagcacgctgcctgccaaagtagtcctggcagctggaccatctctgtaggatcgtaaaaaaatagaaaaaaagaaaaaaaaaagaaagaaagagggaaaaagagctggtggtttgatcatttctgccatgatgtttacaagatggcgaccaccaaagtcaaacgactaacctatctatgaacaacagtagtttctcagggtcactgtccttgaacccaacagtcccttatgagcgtcactgcccaccaaaggtcaatgtcaagagaggaagagagggaggaggggtaggactgcaggggccactccaaactcgcttaggtagaaactattggtgcttgactctcactaggctaaactcaagatttgaccaaatcgagtgatagggatcctggtgggaggagagagggcacatctccagaaaaatgaaaagcaatacaactttaccataaagcctttaaaaccagtaacgtgctgctcaaggaccaagagcaattgcagcagacccagcagcagcagcagcagcacaaacattgctgcctttgtccccacacagcctctaagcgtgctgacatcagattgttaagggcatttttatactcagaactgtcccatccccaggtccccaaacttatggacactgccttagcctcttggaaatcaggtagaccatattctaagttagactcttcccctccctcccacacttcccacccccaggcaaggctgacttctctgaatcagaaaagctattaaagtttgtgtgttgtgtccattttgcaaacccaactaagccaggaccccaatgcgacaagtagttcatgagtattcctagcaaatttctctctttcttcagttcagtagatttccttttttcttttctttttttttttttttttttttggctgtgacctcttcaaaccgtggtacccccccttttctccccacgatgatatctatatatgtatctacaatacatatatctacacatacagaaagaagcagttctcacaatgttgctagttttttgcttctctttcccccaccctactccctccaattcccccttaaacttccaaagcttcgtcttgtgtttgctgcagagtgattcgggggctgacctagaccagtttgcatgattcttctcttgtgatttggttgcactttagacatttttgtgccattatatttgcattatgtatttataatttaaatgatatttaggtttttggctgagtactggaataaacagtgagcatatctggtatatgtcattatttattgttaaattacatttttaagctccatgtgcatataaaggttatgaaacatatcatggtaatgacagatgcaagttattttatttgcttatttttataattaaagatgccatagcataatatgaagcctttggtgaattccttctaagataaaaataataataaagtgttacgttttattggtttcaaaaaaaaaaaaaaaaaaaa

SEQ ID NO: 15 - DNA, 1906 bp, Human
aaagcaaggatgagtcaagctgcgggtgatccaaacaaacactgtcactctttaaaagctgcgctcccgaggttggacctacaaggaggcaggcaagacagcaaggcatagagacaacatagagctaagtaaagccagtggaaatgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgaaagagatatgtagaaggcacaatatgggcactttaaatgaagctaataattcttcacctaagtctctgtgaattgaaatgttcgttttctcctgcctgtgctgtgactcgagtcacactcaagggaacttgagcgtgaatctgtatcttgccggtcatttttatgttattacagggcattcaaatgggctgctgcttagcttgcaccttgtcacatagagtgatctttcccaagagaaggggaagcactcgtgtgcaacagacaagtgactgtatctgtgtagactatttgcttatttaataaagacgatttgtcagttattttatctt

SEQ ID NO: 16 - DNA, 2276 bp, Human
aagcccagcagccccggggcggatggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctctgaccatggcttggatgccctcaggggtgctgacccctgccaggccacgaatatcaggctagagacccatggccatctttgtggctgtgggcaccaggcatgggactgagcccatgtctcctcagggggatggggtggggtacaaccaccatgacaactgccgggagggccacgcaggtcgtggtcacctgccagcgactgtctcagactgggcagggaggctttggcatgacttaagaggaagggcagtcttgggcccgctatgcaggtcctggcaaacctggctgccctgtctccatccctgtccctcagggtagcaccatggcaggactgggggaactggagtgtccttgctgtatccctgttgtgaggttccttccaggggctggcactgaagcaagggtgctggggccccatggccttcagccctggctgagcaactgggctgtagggcagggccacttcctgaggtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctccaaagccattgtaaatgtgtgtacagtgtgtataaaccttcttcttctttttttttttttaaactgaggattgtcattaaacacagttgttttctaaaaaaaaaaaaaaaa

SEQ ID NO: 17 - DNA, 3000 bp, Human
ctggaaccatggagctcagcgtcctcctcttccttgcactcctcacaggcctcttgctactcctggttcagcgtcaccctaactcccatggcaccctcccaccagggccccgccctctgccccttttggggaaccttctgcagatggacagaagaggcctactcaaatcctttctgaggttccgagagaaatatggggacgtcttcacggtacacctgggaccgaggcccgtggtcatgctgtgtggagtagaggccatacgggaggccctggtggacaacgctgaggccttctctggccggggaaaaatcgtcatcatggacccagtctaccagggatatggcatgctctttgccaatggaaaccgctggaaggtgcttcggcgattctctgtgaccaccatgagggacttcgggatgggaaagcggagtgtggaggagcggattcaggacgaggctcagtgtctgatagaggaacttcggaaatccaagggagccctcgtggaccccaccttcctcttccattccattaccgccaacatcatctgctccatcatctttggaaaacgcttccactaccaagatcaagagttcctgaagacgctgaacttgttctgccagagtttcttactcatcagctctatatccagccagctgtttgagctcttctctggcttcttgaaatactttcctggggcacacaggcaagtttacaaaaacctacaggaaatcaatgcttacattggccacagtgtggagaagcaccgtgaaaccctggaccccagcgcccccagggacctcatcgacacctacctgctccacatggaaaaagagaaatccaacccacacagtgaattcagccaccagaacctcatcatcaacacgctctcgctcttctttgctggcactgagaccaccagcaccactctccgctacggcttcctgctcatgctcaaataccctcatgtcgcagagagagtctacaaggagattgaacaggtggttggcccacatcgccctccagcgcttgatgaccgagccaaaatgccatacacagaggcagtcatccgtgagattcagagatttgctgaccttctccccatgggtgtgccccacattgtcacccaacacaccagcttctgagggtacaccatccccaaggacacggaagtatttctcatcctgagcactgctctccgtgacccacactactttgaaaaaccagacgccttcaatcctgaccactttctggatgccaatggggcactgaaaaagaatgaagcttttatccccttctccttagggaagcggatttgtcttggtgaaggcattgcccgtgcggaattgttcctcttcttcaccaccatcctccagaacttctccgtggccagccccgtggctcctgaagacatcgatctgacaccccaggagtgtggtgtgggcaaaatacccccaacataccagatctgcttcctgccccgctgaaggggctgagggaagggggtcaaaggattccagggtcattcagtgtccccacctctgtagataatggctctgactccctgcaacttcctgcctctgagagacctgctgcaagccagcttccttcccttccatggcaccagttgtctgaggtcgcagtgcaaatgagtggaggagtgagattattgaaaattataatatacaaaattatatatatatattttgagacagagtctcactcagttgcccaggctggagtgcagtggcgtgatctcggctcactgcaacctccacccccggggttcaagaaattctcctgcctcagcctccctagtagctgggattacaggtgtgtgctaccatgcctggctaatttttgtatttttagtagagatggggtttcaccgtgttggccaggctgatctcaaactcctgaactcaagtgattcacccaccttagcctcccaaagtgctgggattacaggtgtgagtcaccatgcccggccatgtatatatataattttaaaaattaagatgaaattcacataaaataaaattagccattttaaagtgtacaatttagtggtgtgtggttcattcacaaagctgtacaaccaccaccatctagttccaaacattttctttttttctgagacggagtctcactctgtcacccaggttcgagttcagtggtcttgaactcctgatgtcaggtgattctcctagttccaaatgttttcattatctccccccaacaaaacccatacctatcaagctgtcactccccataccccattctctttttcatctcagcccctgtcaatctggtttttgtccttatggacttaccaattctgaatatttcctataaacagaatcacacaatatttgattttttttttaaaactaagccttgctctgtctcccaggctggagtgctgtggcgtgattttggttcactgcaacctccgccttccaagttcaagagattctcctgcctcagcttccaagtagctgggattacaggcatgtggtaccacgcctggctaattttcttgtatttttagtagggacatgttggccaggctggttgtgagctcctggcctcaggtgatccacacgcctcagtgtcccagagtgctgatattacaggcgtaatatgtgatcttttgtgtctggttcctttcacgttgaacgctatttttgaggttcgtgcctgttgtagaccacagtcacacactgctgtagtcttcccccatcctcattcccagctgcctcctcctactgtttccctctatcaaaaagcctccttggcgcaggttccctgagctgtgggattctgcactggtgctttggattccctgatatgttccttcaaatccactgagaattaaataaacatcgctaaagcatgacctccccacgtcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - DNA, 1016 bp, Lactobacillus gasseri
caatggacgcaagtctgatggagcaacgccgcgtgagtgaagaagggtttcgactcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaagcctaagagattaggagttcccttcggggacgctgagacaggtggtgcatggctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaaccttcgaaggcaagcggatctctgaaagccgttctcagttcggactgtaggctgcaactcgcctacacgaagctggaatcgctagtaatcgcggatcagcacgccgcggtgaatacgttcccggg

SEQ ID NO: 19 - DNA, 1320 bp, Lactobacillus crispatus
  misc_feature (183)..(183): n is a, c, g, or t
cggcgtgcctaatacatgcaagtcgagcgagcggaactaacagatttacttcggtaatgacgttaggaaagcgagcggcggatgggtgagtaacacgtggggaacctgccccatagtctgggataccacttggaaacaggtgctaataccggataagaaagcagatcgcatgatcagcttttnaaaggcggcgtaagctgtcgctatgggatggccccgcggtgcattagctagttggtaaggtaaaggcttaccaaggcgatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgccatttgtagagatacaaagttcccttcggggacgctaagacaggtggtgcatggctgtcgtcagctcgtgtcgtgagatgttgggttaagtcccgcaacgagcgcaacccttgttattagttgccagcattaagttgggcactctaatgagactgccggtgacaaaccggaggaaggtggggatgacgtcaagtcatcatgccccttatgacctgggctacacacgtgctacaatgggcagtacaacgagaagcgagcctgcgaaggcaagcgaatctctgaaagctgttctcagttcggactgcagtctgcaactcgactgcacgaagctg

SEQ ID NO: 20 - DNA, 20 bp, Human
actgctgtcaatgccctgtg

SEQ ID NO: 21 - DNA, 20 bp, Human
accttcttgccatgagcctt

SEQ ID NO: 22 - DNA, 20 bp, Human
aactggacactcaggaccac

SEQ ID NO: 23 - DNA, 24 bp, Human
ggatgtctgggtcttcatattcct

SEQ ID NO: 24 - DNA, 23 bp, Human
cagacaaatgatacgcacaaacg

SEQ ID NO: 25 - DNA, 21 bp, Human
ccaataacaccagccatcacc

SEQ ID NO: 26 - DNA, 22 bp, Human
ctctcaagaccaggaacgagaa

SEQ ID NO: 27 - DNA, 24 bp, Human
gggcagattcaggtattggaatag

SEQ ID NO: 28 - DNA, 24 bp, Human
aagcatcattcacatcgaggctat

SEQ ID NO: 29 - DNA, 26 bp, Human
atgcggtatgacaaatgagaatacac

SEQ ID NO: 30 - DNA, 24 bp, Human
cttgagtaaaagagaacccagcca

SEQ ID NO: 31 - DNA, 22 bp, Human
ttctggaactggctgataaggg

SEQ ID NO: 32 - DNA, 23 bp, Human
gccaggtacagatgctgtcgcag

SEQ ID NO: 33 - DNA, 22 bp, Human
gtgtcttctacatctcggtctg

SEQ ID NO: 34 - DNA, 22 bp, Human
gatgacgccaatcgcaattacc

SEQ ID NO: 35 - DNA, 22 bp, Human
ccttctgctgttcttgttgctg

SEQ ID NO: 36 - DNA, 18 bp, Human
cgtgaggagcctgagcga

SEQ ID NO: 37 - DNA, 16 bp, Human
cgatgctgccgcctgt

SEQ ID NO: 38 - DNA, 21 bp, Human
ttctctccatcgccttgtctg

SEQ ID NO: 39 - DNA, 20 bp, Human
agtgtgcccatccatgactg

SEQ ID NO: 40 - DNA, 24 bp, Human
ctttgccaccttcgtgactttatg

SEQ ID NO: 41 - DNA, 20 bp, Human
acagttgtcagtctgccact

SEQ ID NO: 42 - DNA, 17 bp, Human
tgagaaaggccagggcg

SEQ ID NO: 43 - DNA, 21 bp, Human
aatcgaagcctgtcacactgc

SEQ ID NO: 44 - DNA, 25 bp, Human
cccactctacaactcattcacagag

SEQ ID NO: 45 - DNA, 20 bp, Human
ggttcctcagtagaggcagg

SEQ ID NO: 46 - DNA, 22 bp, Human
ctgcccaatcacttctccaaca

SEQ ID NO: 47 - DNA, 21 bp, Human
tttctccatcaggctgtctct

SEQ ID NO: 48 - DNA, 17 bp, Human
ccatgcctatgcccctg

SEQ ID NO: 49 - DNA, 22 bp, Human
gtccctgttgtatcctttgtcc

SEQ ID NO: 50 - DNA, 20 bp, Human
caagactcaccgagaagggg

SEQ ID NO: 51 - DNA, 19 bp, Human
gccttggctgctgttgtgt

SEQ ID NO: 52 - DNA, 25 bp, Human
ccgtgagattcagagatttgctgac

SEQ ID NO: 53 - DNA, 24 bp, Human
tgagaaatacttccgtgtccttgg

SEQ ID NO: 54 - DNA, 19 bp, Lactobacillus gasseri
cagagcaagcggaagcaca

SEQ ID NO: 55 - DNA, 21 bp, Lactobacillus gasseri
ttgcttacttactgctccccg

SEQ ID NO: 56 - DNA, 19 bp, Lactobacillus crispatus
gagaaagccaagcggaagc

SEQ ID NO: 57 - DNA, 21 bp, Lactobacillus crispatus
ttgcttacttactgctccccg

(19) United States
(12) Patent Application Publication
(10) Pub. No.: US 2020/0270684 A1
(43) Pub. Date: Aug. 27, 2020

(54) METHOD FOR BODY FLUID IDENTIFICATION

(71) Applicant: INSTITUTE OF ENVIRONMENTAL SCIENCE AND RESEARCH LIMITED, Wellington (NZ)

(72) Inventors: Patricia ALBANI, Wellington (NZ); Rachel FLEMING, Wellington (NZ); Jayshree PATEL, Wellington (NZ)

(73) Assignee: INSTITUTE OF ENVIRONMENTAL SCIENCE AND RESEARCH LIMITED, Wellington (NZ)

(21) Appl. No.: 16/652,503

(22) PCT Filed: Oct. 2, 2018
(86) PCT No.: PCT/NZ2018/050133

§ 371 Date: Mar. 31, 2020

(30) Foreign Application Priority Data
Oct. 2, 2017 (NZ) 735997
Feb. 9, 2018 (NZ) 739809

(51) Int. Cl. C12Q 1/6881; C12Q 1/6879

(52) U.S. Cl. CPC C12Q 1/6881; C12Q 2600/158; C12Q 2600/16; C12Q 1/6879

(57) ABSTRACT

Crime scene investigators need to identify biological tissue or fluid types. Such analysis is typically done using conventional chemical, serological and enzymatic tests to identify the body fluid or tissue, however, these tests can be unreliable and often do not meet the specificity and sensitivity required for forensic analysis. The present invention provides a method for accurately identifying circulatory blood, saliva, spermatozoa, seminal fluid, menstrual fluid and vaginal material by detection of specific RNA sequences. In particular, the invention provides a method for determining the type of a biological sample, comprising the steps of detecting RNA from the sample associated with any one or more of HBD, SLC4A1, GYPA, FDCSP, HTN3, STATH, PRM1, TNP1, PRM2, KLK2, MSMB, TGM4, MMP10, STC1, MMP3, MMP11, CYP2B7P, Lactobacillus gasseri (L.gass) and Lactobacillus crispatus {L.crisp) and determining whether the sample is circulatory blood, saliva, spermatozoa, seminal fluid, menstrual fluid or vaginal material.

### RELATED APPLICATIONS

This application claims priority to New Zealand Provisional Application No. 735997 filed on 2 Oct. 2017 and New Zealand Provisional Application No. 739809 filed on 9 Feb. 2018, the entire teachings of which are incorporated herein by reference.

### TECHNICAL FIELD

The technical field is the detection of RNA sequences, and the use of these sequences for identification and typing of samples, in particular samples containing degraded RNA.

### BACKGROUND

In many instances, crime scene investigators come across cellular or body fluids of interest, but need to identify what tissue or fluid it is. This information can be critical in establishing activity scenarios of a case. For example, the presence of menstrual blood may indicate sexual activity, whereas circulatory blood may be the result of a traumatic injury. Such analysis is typically done using conventional chemical, serological and enzymatic tests to identify the body fluid or tissue, however, these tests can be unreliable and often do not meet the specificity and sensitivity required for forensic analysis.

Messenger RNA (mRNA) profiling based on unique gene expression patterns in cells and tissues has emerged as a method to overcome these limitations [1-4]. DNA/RNA co-extraction for combined short tandem repeat (STR) and body fluid profiling is now an effective and comprehensive tool used by casework laboratories around the world. Yet since the introduction of differentially expressed mRNAs for forensic saliva analysis in 2003 [2], only a small set of ‘core’ markers has been used for multiplex design. These include histatin 3 (HTN3) and statherin (STATH) for saliva and buccal mucosa [1,3,5-7], protamines 1 and 2 (PRM1/2) for semen [1,3,5-7], transglutaminase 4 (TGM4) or semenogelin 1 (SEMG1) for seminal fluid [1,3], matrix metallopeptidases (MMPs) 7, 10 or 11 for menstrual fluid [1,3,5-7], as well as human beta-defensin 1 (HBD1), mucin 4 (MUC4) or Lactobacilli crispatus (L.crisp) and gasseri (L.gass) for vaginal material [1,3,5-7]. Greater variability is seen in the use of circulatory blood markers. Commonly targeted transcripts include spectrin beta (SPTB), hydroxymethylbilane synthase (PBGD), 5′-aminolevulinate synthase 2 (ALAS2), glycophorin A (GYPA), adhesion molecule, interacts with CXADR antigen 1 (AMICA1), CD93 molecule and haemoglobin beta (HBB) [1,3,5-7]. Other mRNA markers have been proposed, but are less frequently used due to inferior specificity and sensitivity in comparison to the above markers [8-13]. An exception to this is cytochrome P450 family 2, subfamily B, member 7, pseudogene (CYP2B7P), a useful marker for the detection of vaginal material [14].

The ability to accurately detect and quantify RNA abundance is a fundamental capability in molecular biology. The broad set of RNA detection methods currently available range from non-amplification methods (in situ hybridization, microarray and NanoString nCounter), to amplification (PCR) based methods (reverse transcriptase PCR (RT-PCR) and quantitative reverse transcriptase PCR (qRT-PCR)). With the exception of RNAseq (next generation sequencing, also referred to as second generation sequencing or massively parallel sequencing), a key prerequisite of all RNA detection technology is prior knowledge of the target RNA sequence. This targeting is facilitated by oligonucleotide sequences in both non-amplification methods (probe) and amplification-based methods (primers).

Methods for PCR primer design are always evolving [1, 2] but remain based around the core criteria of specificity, thermodynamics, secondary structure, dimerisation and amplicon length [3-7]. In addition to these criteria, RT-PCR primer design (for RNA amplification) also considers exon boundary coverage to ensure amplification of only cDNA and avoid amplification of genomic DNA [8]. Amongst other experimental factors [9-14], it is widely acknowledged that PCR primer design has critical implications to target amplification, detection and quantification [3, 8, 11, 15-18].

Whilst improvements to primer design can yield performance improvements, the target molecule must also be considered. RNA is unstable and easily degraded [19-22]. Conventional methodology recommends sample RNA integrity (RIN) to be at least RIN 8 or above to ensure proper performance [23-26]. RIN values range from 10 (intact) to 1 (totally degraded). The gradual degradation of RNA is reflected by a continuous shift towards shorter RNA fragments the more degraded the RNA is. In this context shorter means that the RNA fragments are not as long as non-degraded RNA and over time the RNA fragments break down into smaller and smaller fragments.

Furthermore, a degree of degradation is unavoidable in situations where real-world samples must be analysed—forensic, clinical, FFPE and environmental sampling. The detrimental effects of RNA degradation on RNA detection and quantification are well documented [24, 27-30]. Currently there is no clear solution to this problem except to avoid analysing degraded RNA.

Here the inventors have established a method for accurately identifying circulatory blood, saliva, spermatozoa, seminal fluid, menstrual fluid and vaginal material by detection of specific RNA sequences.

It is an object of the invention to provide improved methods and/or materials for specific detection of tissues types in unknown samples and/or at least to provide the public with a useful choice.

### SUMMARY OF THE INVENTION

### Typing a Sample

In a first aspect the invention provides a method of typing a sample, the method comprising the steps of detecting an RNA sequence in a sample by a method of the invention, wherein detecting the RNA sequence marker indicates the type of sample.

The method may involve using just one pair of primers, or a single probe, to type the sample. Alternatively multiple pairs of primers, or multiple probes, may be used.

Specifically, the invention provides for a method for determining the type of a biological sample, comprising the steps of detecting RNA from the sample associated with any one or more of HBD, SLC4A1, GYPA, FDCSP, HTN3, STATH, PRM1, TNP1, PRM2, KLK2, MSMB, TGM4, MMP10, STC1, MMP3, MMP11, CYP2B7P, L.gass and L.crisp and establishing whether the sample is circulatory blood, saliva, spermatozoa, seminal fluid, menstrual fluid or vaginal material.

The method includes detecting whether a biological sample is circulatory blood, comprising the step of detecting RNA associated with HBD, SLC4A1 and/or GYPA.

The method includes detecting whether a biological sample is saliva, comprising the step of detecting RNA associated with FDCSP and/or HTN3 and/or STATH.

The method includes detecting whether a biological sample is spermatozoa, comprising the step of detecting RNA associated with PRM1, TNP1 and/or PRM2.

The method includes detecting whether a biological sample is seminal fluid, comprising the step of detecting RNA associated with KLK2, MSMB and/or TGM4.

The method includes detecting whether a biological sample is menstrual fluid, comprising the step of detecting RNA associated with MMP10 and/or STC1 and/or MMP3 and/or MMP11.

The method includes detecting whether a biological sample is vaginal material, comprising the step of detecting RNA associated with CYP2B7P, L.gass and/or L.crisp.

The method of the present invention includes, but is not limited to the use of multiplex PCR.

### Typing Sample by Multiplex PCR

In one embodiment multiplex PCR is performed with one or more primers, at least one of which is diagnostic for the type of sample.

Preferably the method includes the use of one or more primers specific for any one of HBD, SLC4A1, GYPA, FDCSP, HTN3, STATH, PRM1, TNP1, PRM2, KLK2, MSMB, TGM4, MMP10, STC1, MMP3, MMP11, CYP2B7P, L.gass or L.crisp, more preferably the primers are selected from anyone of SEQ ID Nos: 20 to 57.

The method includes detecting whether a biological sample is circulatory blood, comprising the step of detecting RNA associated with HBD using primers of SEQ ID No: 20 and 21, and/or SLC4A1 using primers of SEQ ID No:22 and 23 and/or GYPA using primers of SEQ ID No: 24 and 25.

The method includes detecting whether a biological sample is saliva, comprising the step of detecting RNA associated with FDCSP using primers of SEQ ID No: 26 and 27, and/or HTN3 using primers of SEQ ID No: 28 and 29 and/or STATH using primers of SEQ ID NO: 30 and 31.

The method includes detecting whether a biological sample is spermatozoa, comprising the step of detecting RNA associated with PRM1 using primers of SEQ ID No:32 and 33 and/or TNP1 using primers of SEQ ID No:34 and 35 and or PRM2 using primers of SEQ ID No: 36 and 37.

The method includes detecting whether a biological sample is seminal fluid, comprising the step of detecting RNA associated with KLK2 using primers of SEQ ID No:38 and 39, and/or MSMB using primers of SEQ ID No:40 and 41 and/or TGM4 using primers of SEQ ID No: 42 and 43.

The method includes detecting whether a biological sample is menstrual fluid, comprising the step of detecting RNA associated with MMP10 using primers of SEQ ID No:44 and 45, and/or STC1 using primers of SEQ ID No:446 and 47 and/or MMP3 using primers of SEQ ID No:48 and 49 and/or MMP11 using primers of SEQ ID NO: 50 and 51.

The method includes detecting whether a biological sample is vaginal material, comprising the step of detecting RNA associated with CYP2B7P using primers of SEQ ID No:52 and 53 and/or L.gass using primers of SEQ ID No: 54 and 55 and/or L.crisp of SEQ ID No: 56 and 57.

### Primers

In a further embodiment the invention provides a primer capable of hybridising to the stable region of the RNA sequence, or a cDNA corresponding to the stable region or a complement thereof.

In a further embodiment the invention provides a primer comprising a sequence of at least 5 nucleotides with at least 70% identity to any part of the sequence of any one of SEQ ID NO:1 to 19 or a complement thereof.

In a further embodiment the primer consists of a sequence of at least 5 nucleotides with at least 70% identity to the sequence of any one of SEQ ID NO:1 to 19, or a complement thereof.

In a further embodiment the primer comprises a sequence of at least 5 nucleotides of the sequence of any one of SEQ ID NO:1 to 19, or a complement thereof.

In a further embodiment the primer consists of a sequence of at least 5 nucleotides of the sequence of any one of SEQ ID NO:1 to 19, or a complement thereof.

In a further embodiment the primer comprises a sequence selected from the group comprising SEQ ID NO:20 to SEQ ID NO: 57, or a complement of any one thereof.

In a further embodiment the primer consists of a sequence selected from the group comprising SEQ ID NO:20 to SEQ ID NO: 57, or a complement of any one thereof.

In a further embodiment the primer is selected from the group comprising SEQ ID NO:20 to SEQ ID NO: 57, or a complement of any one thereof.

In a further embodiment the primer includes an attached label or tag.

In a further embodiment the labelled or tagged primer is not found in nature.

The primers of the invention can be used on microarrays or chips or like products for the detection of RNA sequences.

### Kit of Primers

In a further embodiment the invention provides a kit comprising at least one primer of the invention.

Preferably the kit comprises at least one primer pair selected from SEQ ID Nos: 20 and 21, 22 and 23, 24 and 25, 26 and 27, 28 and 29, 30 and 31, 32 and 33, 34 and 35, 36 and 37, 38 and 39, 40 and 41, 42 and 43, 44 and 45, 46 and 47, 48 and 49, 50 and 51, 52 and 53, 54 and 55, and 56 and 57.

In one embodiment the kit also comprises instructions for use.

### Probes

In a further embodiment the invention provides a probe capable of hybridising to the RNA sequence, or a corresponding cDNA or a complement thereof. Preferably the probe is capable of hybridising to any one of HBD, SLC4A1, GYPA, FDCSP, HTN3, PRM1, TNP1, PRM2, KLK2, MSMB, TGM4, MMP10, STC1, MMP3, CYP2B7P, L.gass and L.crisp.

In a further embodiment the invention provides a probe comprising a sequence of at least 10 nucleotides with at least 70% identity to any part of the sequence of any one of SEQ ID NO:1 to 19 or a complement thereof.

In a further embodiment the probe consists of a sequence of at least 10 nucleotides with at least 70% identity to the sequence of any one of SEQ ID NO:1 to 19, or a complement thereof.

In a further embodiment the probe comprises a sequence of at least 10 nucleotides of the sequence of any one of SEQ ID NO:1 to 19, or a complement thereof.

In a further embodiment the probe consists of a sequence of at least 10 nucleotides of the sequence of any one of SEQ ID NO:1 to 19, or a complement thereof.

In a further embodiment the probe includes an attached label or tag.

In a further embodiment the labelled or tagged probe is not found in nature.

The primers of the invention can be used on microarrays or chips or like products for the detection of RNA sequences.

### Kit of Probes

In a further embodiment the invention provides a kit comprising at least one probe of the invention.

Preferably the kit comprises at least 2, more preferably at least 3, more preferably at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 probes, more preferably at least 31 probes, more preferably at least 32 probes, more preferably at least 33 probes, more preferably at least 34, more preferably at least 35, more preferably at least 36, more preferably at least 37, more preferably at least 38 probes of the invention.

In one embodiment the kit also comprises instructions for use.

### MicroArrays

In another aspect the invention provides a microarray comprising a sequence of at least 5 nucleotides with at least 70% identity to any part of the sequence of any one of SEQ ID NO:1 to SEQ ID NO:19 or a complement thereof.

In another aspect the invention provides a microarray comprising a sequence of at least 5 nucleotides of a sequence of any one of SEQ ID NO:1 to SEQ ID NO:19 or a complement thereof.

In another aspect the invention provides a microarray comprising a sequence of at least 10 nucleotides of a sequence with at least 70% identify to any part of the sequence of any one of SEQ ID NO:1 to SEQ ID NO:19 or a complement thereof.

In another aspect the invention provides a microarray comprising a sequence of at least 10 nucleotides of a sequence of any one of SEQ ID NO:1 to SEQ ID NO:19 or a complement thereof.

Preferably the sequence comprises at least 5, more preferably at least 10, more preferably at least 15, more preferably at least 20, more preferably at least 25, more preferably at least 30, more preferably at least 35, more preferably at least 40, more preferably at least 45, more preferably at least 50, more preferably at least 55, more preferably at least 60, more preferably at least 65, more preferably at least 70, more preferably at least 75, more preferably at least 80, more preferably at least 85, more preferably at least 90, more preferably at least 95, more preferably at least 100, more preferably at least 120, more preferably at least 140, more preferably at least 160, more preferably at least 180, more preferably at least 200, more preferably at least 240, more preferably at least 250 nucleotides of the sequences of the invention.

Those skilled in the art would understand how to select the appropriate probes or primers for detecting any of the listed markers, based on the information in the Sequence Listing, and elsewhere in the specification.

It will be understood to those skilled in the art that a probe or primer can be produced that can hybridise to any part of a stable region. The probes and primers mentioned herein are given as examples only to demonstrate that the stable regions can be used to identify and type degraded RNA. Any primer or probe that is complementary to the stable region would be suitable in the methods of the invention.

The present invention therefore provides:

1. A method for determining the type of a biological sample, comprising the steps of detecting RNA from the sample associated with any one or more of HBD, SLC4A1, GYPA, FDCSP, HTN3, STATH, PRM1, TNP1, PRM2, KLK2, MSMB, TGM4, MMP10, STC1, MMP3, MMP11, CYP2B7P, Lactobacillus gasseri (L.gass) and Lactobacillus crispatus (L.crisp) and determining whether the sample is circulatory blood, saliva, spermatozoa, seminal fluid, menstrual fluid or vaginal material.
2. The method of 1, comprising detecting an RNA associated with one or more of SEQ ID Nos: 1 to 19.
3. The method of 1 or 2, wherein the step of detecting the RNA includes the use of one or more primers specific for any one or more of HBD, SLC4A1, GYPA, FDCSP, HTN3, STATH, PRM1, TNP1, PRM2, KLK2, MSMB, TGM4, MMP10, STC1, MMP3, MMP11, CYP2B7P, Lactobacillus gasseri (L.gass) and Lactobacillus crispatus (L.crisp).
4. The method of 3, wherein the one or more primers are selected from SEQ ID Nos: 20 to 57.
5. The method of any one of 1 to 4, comprising determining if the biological sample is circulatory blood, comprising the step of detecting RNA associated with HBD using primers of SEQ ID No: 20 and 21, and/or SLC4A1 using primers of SEQ ID No:22 and 23 and/or GYPA using primers of SEQ ID No: 24 and 25.
6. The method of any one of 1 to 4, comprising determining if the biological sample is saliva, comprising the step of detecting RNA associated with FDCSP using primers of SEQ ID No: 26 and 27, and/or HTN3 using primers of SEQ ID No: 28 and 29, and/or STATH using primers of SEQ ID No: 30 and 31.
7. The method of any one of 1 to 4, comprising determining if the biological sample is spermatozoa, comprising the step of detecting RNA associated with PRM1 using primers of SEQ ID No:32 and 33 and/or TNP1 using primers of SEQ ID No:34 and 35 and or PRM2 using primers of SEQ ID No: 36 and 37.
8. The method of any one of 1 to 4, comprising determining if the biological sample is seminal fluid, comprising the step of detecting RNA associated with KLK2 using primers of SEQ ID No:38 and 39, and/or MSMB using primers of SEQ ID No:40 and 41 and/or TGM4 using primers of SEQ ID No: 42 and 43.
9. The method of any one of 1 to 4, comprising determining if the biological sample is menstrual fluid, comprising the step of detecting RNA associated with MMP10 using primers of SEQ ID No:44 and 45, and/or STC1 using primers of SEQ ID No:46 and 47 and/or MMP3 using primers of SEQ ID No:48 and 49 and/or MMP11 using primers of SEQ ID No. 50 and 51.
10. The method of any one of 1 to 4, comprising determining if the biological sample is vaginal material, comprising the step of detecting RNA associated with CYP2B7P using primers of SEQ ID No:52 and 53 and/or L.gass using primers of SEQ ID No: 54 and 55 and/or L.crisp of SEQ ID No: 56 and 57.
11. The method of any one of 1 to 10, comprising testing for the presence of RNA of all of HBD, SLC4A1, GYPA, FDCSP, HTN3, STATH, PRM1, TNP1, PRM2, KLK2, MSMB, TGM4, MMP10, STC1, MMP3, MMP11, CYP2B7P, Lactobacillus gasseri (L.gass) and Lactobacillus crispatus (L.crisp) in the biological sample.
12. The method of any one of 1 to 11, comprising detecting the presence of RNA of any one or more of HTN3 and FDCSP; and/or SLC4A1, HBD, STC1 and MMP10 and/or TNP1, PRM1, KLK2, MSMB and CYP2B79.
13 The method of any one of 1 to 12, wherein the primer is labelled.
14. The method of claim 13, wherein the primer is labelled with a fluorescence label, biotin, radioactive or non-radioactive label.
15. The method of any one of 1 to 14, wherein the RNA is detected using an amplification method.
16. The method of 15, wherein the amplification method is selected from the group comprising polymerase chain reaction (PCR), reverse transcriptase PCR (RT-PCR), quantitative reverse transcriptase PCR (qRT-PCR), multiplex PCR, multiplex ligation-dependent probe amplification (MLPA) or quantitative PCR (Q-PCR).
17. A kit for use in the method of any one of 1 to 16, the kit comprising at least one primer pair selected from SEQ ID Nos: 20 and 21, 22 and 23, 24 and 25, 26 and 27, 28 and 29, 30 and 31, 32 and 33, 34 and 35, 36 and 37, 38 and 39, 40 and 41, 42 and 43, 44 and 45, 46 and 47, 48 and 49, 50 and 51, 52 and 53, 54 and 55, and 56 and 57.

Those skilled in the art will understand the relationship between marker genes, the mRNA encoded by the marker genes, and the stable regions within the mRNA. Those skilled in the art will understand that the sequences presented are DNA sequences corresponding to the mRNA or stable regions within the mRNA.

### DETAILED DESCRIPTION OF THE INVENTION

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner. However, in preferred embodiments comprising can be replaced with consisting.

As used here, the term “RNA” means messenger RNA, small RNA, microRNA, non-coding RNA, long non-coding RNA, small non-coding RNA, ribosomal RNA, small nucleolar RNA, transfer RNA and all other RNA species and sequences.

As used herein, the term “stable region” means a region or regions in an RNA sequence which has more aligned sequencing reads than another region, or regions, of the same RNA sequence.

As used herein the term “degraded RNA” refers to is RNA that is no longer intact. In other words, the theoretical full length RNA, as annotated or predicted in sequence databases, is no longer intact. The full length RNA may be fragmented and/or some nucleotides are no longer present. This may occur at any position along the RNA sequence.

The inventors stress that how the level of RNA degradation is measured is not essential and the invention lies in that the method is also suitable for use on samples where there may be some degree of degraded RNA.

The present inventors have identified a method to identify the type of biological sample, with the aim that the method can be used to identify biological samples obtained in the forensic situation. Specifically, the method can be utilized to determine whether a given biological sample is circulatory blood, saliva, spermatozoa, seminal fluid, menstrual fluid or vaginal material.

The invention comprises determining the presence of RNA for markers that the inventors have identified as being specific for circulatory blood, saliva, spermatozoa, seminal fluid, menstrual fluid and/or vaginal material. As shown in Table 1, in order to identify circulatory blood, markers HBD and/or SLC4A1 and/or GYPA can be utilized; for saliva, markers FDCSP and/or HTN3 can be utilized; for spermatozoa, markers PRM1 and/or TNP1 and/or PRM2 can be utilized; for seminal fluid, markers KLK2 and/or MSMB and/or TGM4 can be utilized; for menstrual fluid, markers MMP10, MMP3 and/or STC1 can be utilized; and for vaginal material marker CYP2B7P and/or L.gass and/or L.crisp can be utilized.

It will be appreciated that a single marker or pair of markers specific for a particular type can be utilized to test for whether a given sample is that type. Alternatively one or pairs of specific markers can be utilized in order to determine whether a given sample is one or two or more types. The invention can also be used where the presence of RNA of all of the markers HBD, SLC4A1, GYPA, FDCSP, HTN3, PRM1, TNP1, PRM2, KLK2,TGM4, MSMB, MMP10, STC1, MMP3, CYP2B7P, L.gass and L.crisp are tested in the sample in order to establish if the sample is circulatory blood, saliva, spermatozoa, seminal fluid, menstrual fluid and/or vaginal material.

The method of the invention then involves producing probes or primers targeting the mRNA or stable regions in the mRNA. The method allows for improved detection of such RNA sequences, particularly in samples in which the RNA is, or has been, subjected to degradation.

TABLE 1

| Body fluid | mRNA | Primer sequence (5′ to 3′)¹ | SEQ ID NO: |
|---|---|---|---|
| Circulatory | HBD | F: ACTGCTGTCAATGCCCTGTG | 20 |
| Blood | | R: FAM-ACCTTCTTGCCATGAGCCTT | 21 |
| | SLC4A1 | F: HEX-AACTGGACACTCAGGACCAC | 22 |
| | | R: GGATGTCTGGGTCTTCATATTCCT | 23 |
| | GYPA | F: HEX-CAGACAAATGATACGCACAAACG | 24 |
| | | R: CCAATAACACCAGCCATCACC | 25 |
| Saliva | FDCSP | F: HEX-CTCTCAAGACCAGGAACGAGAA | 26 |
| | | R: GGGCAGATTCAGGTATTGGAATAG | 27 |
| | HTN3 | F: HEX-AAGCATCATTCACATCGAGGCTAT | 29 |
| | | R: ATGCGGTATGACAAATGAGAATACAC | 29 |
| | STATH | F: HEX-CTTGAGTAAAAGAGAACCCAGCCA | 30 |
| | | R: TTCTGGAACTGGCTGATAAGGG | 31 |
| Spermatozoa | PRM1 | F: HEX-GCCAGGTACAGATGCTGTCGCAG | 32 |
| | | R: GTGTCTTCTACATCTCGGTCTG | 33 |
| | TNP1 | F: GATGACGCCAATCGCAATTACC | 34 |
| | | R: FAM-CCTTCTGCTGTTCTTGTTGCTG | 35 |
| | PRM2 | F: FAM-CGTGAGGAGCCTGAGCGA | 36 |
| | | R: CGATGCTGCCGCCTGT | 37 |
| Seminal fluid | KLK2 | F: TTCTCTCCATCGCCTTGTCTG | 38 |
| | | R: HEX-AGTGTGCCCATCCATGACTG | 39 |
| | MSMB | F: CTTTGCCACCTTCGTGACTTTATG | 40 |
| | | R: FAM-ACAGTTGTCAGTCTGCCACT | 41 |
| | TGM4 | F: HEX-TGAGAAAGGCCAGGGCG | 42 |
| | | R: AATCGAAGCCTGTCACACTGC | 43 |
| Menstrual fluid | MMP10 | F: HEX-CCCACTCTACAACTCATTCACAGAG | 44 |
| | | R: GGTTCCTCAGTAGAGGCAGG | 45 |
| | STC1 | F: FAM-CTGCCCAATCACTTCTCCAACA | 46 |
| | | R: TTTCTCCATCAGGCTGTCTCT | 47 |
| | MMP3 | F: FAM-CCATGCCTATGCCCCTG | 48 |
| | | R: GTCCCTGTTGTATCCTTTGTCC | 49 |
| | MMP11 | F: FAM-CAAGACTCACCGAGAAGGGG | 50 |
| | | R: GCCTTGGCTGCTGTTGTGT | 51 |
| Vaginal | CYP2B7P | F: CCGTGAGATTCAGAGATTTGCTGAC | 52 |
| Material | | R: HEX-TGAGAAATACTTCCGTGTCCTTGG | 53 |
| | L.gass | F: FAM-CAGAGCAAGCGGAAGCACA | 54 |
| | | R: TTGCTTACTTACTGCTCCCCG | 55 |
| | L.crisp | F: FAM-GAGAAAGCCAAGCGGAAGC | 56 |
| | | R: TTGCTTACTTACTGCTCCCCG | 57 |

¹Labels (where shown) are optional

### RNA Degradation

Whilst improvements to primer or probe design can yield performance improvements in amplification and hybridization methods, the target molecule must also be considered. RNA is unstable and easily degraded [40-43]. Conventional methodology recommends sample RNA integrity (RIN) to be at least RIN 8 or above to ensure proper performance [44-47].

Other measures of the degradation of RNA sequences are known, such as DV200 [63].

It will appreciated by the skilled person however, that how the level of RNA degradation is measured is not essential and the invention lies in the ability to detect degraded RNA.

A degree of degradation is unavoidable in situations where real-world samples must be analysed—for example, forensic, clinical, Formalin-Fixed Paraffin-Embedded (FFPE) and environmental samples. The detrimental effects of RNA degradation on RNA detection and quantification are well documented [45, 48-51].

The methods and materials of the invention allow for improved detection of RNA sequences of interest, particularly when RNA samples have been degraded. This allows typing of samples that contain degraded RNA, including samples having a RIN value less than 8. This is particularly surprising as prior to the present invention it was generally considered that detection and typing of degraded RNA sequences where RIN was less than 8 was not able to be achieved to an acceptable performance value.

RIN values range from 10 (intact) to 1 (totally degraded). The gradual degradation of RNA is reflected by a continuous shift towards shorter RNA fragments the more degraded the RNA is. Where the RIN value is less than 1, this signifies that RNA is degraded beyond detection.

The inventors have found that while the probes and primers of the invention are useful in detecting and typing the source of degraded RNA including RNA having a RIN value less than 8, the probes and primers of the invention can also be used to detect and type the source of RNA having a RIN value of 8-10. That is, the primers and probes of the invention also allow the detection and typing of RNA irrespective of the RIN value.

In one embodiment the methods of the invention works, or allows for RNA marker detection, when RNA integrity (RIN) is less than RIN 8, more preferably less than RIN 7, more preferably less than RIN 6, more preferably less than RIN 5, more preferably less than RIN 4, more preferably less than RIN 3, more preferably less than RIN 2, more preferably less than 1. The inventors have also found that the methods of the invention can be used to type RNA where RIN is undetermined (beyond detection).

Specifically the inventors have developed a set of primers specific for regions of the 19 markers; HBD, SLC4A1, GYPA, FDCSP, HTN3, STATH, PRM1, TGM4, TNP1, PRM2, KLK2, MSMB, MMP10, STC1, MMP3, MMP11, CYP2B7P. L.gass or L.crisp, specific for circulatory blood, saliva, spermatozoa, seminal fluid, menstrual fluid and vaginal material, which allow identification of samples likely to have undergone a degree of RNA degradation. The corresponding primers are outlined in Table 1.

### Methods for RNA Detection

It will appreciated that any suitable methods of detecting RNA can be utilized in the present invention. Many methods are known in the art and could be utilized in order to identify the origin of a biological sample.

The broad set of RNA detection methods currently available range from non-amplification methods (in situ hybridization, microarray and NanoString nCounter), to amplification (PCR) based methods (reverse transcriptase PCR (RT-PCR) and quantitative reverse transcriptase PCR (qRT-PCR)), next generation sequencing (massively parallel sequencing/high throughput sequencing), and RNA-aptamers.

### In Situ Hybridization

In situ hybridization (ISH) is a type of hybridization that uses a labelled complementary DNA or RNA strand (i.e., probe) to localize a specific DNA or RNA sequence in a portion or section of tissue (in situ), or, if the tissue is small enough (e.g., plant seeds, Drosophila embryos), in the entire tissue (whole mount ISH), in cells, and in circulating tumour cells (CTCs). This is distinct from immunohistochemistry, which usually localizes proteins in tissue sections.

In situ hybridization is a powerful technique for identifying specific mRNA species within individual cells in tissue sections, providing insights into physiological processes and disease pathogenesis. However, in situ hybridization requires that many steps be taken with precise optimization for each tissue examined and for each probe used. In order to preserve the target mRNA within tissues, it is often required that crosslinking fixatives (such as formaldehyde) be used.

Degradation of target RNA is a problem in ISH experiments. The methods of the invention provide a solution to this problem by targeting stable regions within target RNA of interest.

### Microarray

A DNA microarray (also commonly known as DNA chip or biochip) is a collection of microscopic DNA spots attached to a solid surface. Scientists use DNA microarrays to measure the expression levels of large numbers of genes simultaneously or to genotype multiple regions of a genome. Each DNA spot contains picomoles (10⁻¹² moles) of a specific DNA sequence, known as probes (or reporters or oligos). These can be a short section of a gene or other DNA element that is used to hybridize a cDNA or cRNA (also called anti-sense RNA) sample (called target) under high-stringency conditions. Probe-target hybridization is usually detected and quantified by detection of fluorophore-, silver-, or chemiluminescence-labeled targets to determine relative abundance of nucleic acid sequences in the target.

The present invention has application for microarray analysis of tissues, including tissues that are subject to degradation. By designing probes to include on the microarray chip that target stable regions of RNA (according to the present invention), the microarray analysis may provide a more realistic representation of the in vivo expression profile, that is not so skewed by degradation after RNA is extracted from the tissue sample. Such chips would also be able to be used to screen samples containing RNA, including degraded RNA, in order to type the source of that RNA as has been previously described.

NanoString nCounter

NanoString's nCounter technology is a variation on the DNA microarray and was invented and patented by Krassen Dimitrov and Dwayne Dunaway. It uses molecular “barcodes” and microscopic imaging to detect and count up to several hundred unique RNAs in one hybridization reaction. Each color-coded barcode is attached to a single target-specific probe corresponding to a gene of interest.

The NanoString protocol includes the following steps:
Hybridization: NanoString's Technology employs two ˜50 base probes per mRNA that hybridize in solution. The reporter probe carries the signal, while the capture probe allows the complex to be immobilized for data collection.
Purification and Immobilization: After hybridization, the excess probes are removed and the probe/target complexes are aligned and immobilized in the nCounter Cartridge.
Data Collection: Sample Cartridges are placed in the Digital Analyzer instrument for data collection. Color codes on the surface of the cartridge are counted and tabulated for each target molecule.

The nCounter Analysis System: The system consists of two instruments: the Prep Station, which is an automated fluidic instrument that immobilizes CodeSet complexes for data collection, and the Digital Analyzer, which derives data by counting fluorescent barcodes. As the NanoString nCounter system is dependent on probe-target hybridization for RNA detection and analysis, the present invention has immediate application to NanoString nCounter. NanoString nCounter probe design (target hybridization sites) are designed to conform to certain thermodynamic requirements and gives no consideration to target RNA degradation or stability. Therefore we believe that with the present invention NanoString nCounter RNA detection can be vastly improved by designing probes to hybridise to stable regions in the RNA sequence.

### Samples

The sample may be any type of biological sample that includes RNA.

Samples suitable for in situ hybridization include biological tissue sections.

Preferably the forensic sample is selected from the group comprising blood, semen (with or without spermatozoa), saliva, vaginal material and menstrual fluid.

### RNA Extraction

RNA extraction procedures are well known to those skilled in the art. Examples include: Acid guanidium thiocyanate-phenol-chloroform RNA extraction [64]; magnetic bead-based RNA extraction [65]; column-based RNA purification [66,67]; and TRIzol (TRI reagent) RNA extraction [68].

### RNA Sequencing and Stable Region Identification

RNA sequencing refers to sequencing of all RNA in a sample using what is commonly known as Next Generation Sequencing (NGS) (second generation sequencing or massively parallel sequencing; [69-72]). Although different sequencing instrumentation manufacturers employ slightly different sequencing chemistry, RNA sequencing can be achieved using any of these NGS (massively parallel sequencing) technologies [69,73]. As there are many NGS technologies available, there are small differences in the methodology for RNA sequencing. The following is a description of how RNA sequencing using NGS works in general [70]:
Total RNA is extracted from the sample of interest, using a common RNA extraction method. Post-extraction processes can be used to enrich the RNA sample.
Complementary DNA (cDNA) is then synthesised using extracted RNA. cDNA is then used as the template for RNA sequencing.
NGS uses variations of sequencing by synthesis (SBS) chemistry [74]. With cDNA as a template, new nucleotide fragments, known as reads, are synthesised base by base, with each incorporated base recorded during sequencing [74].
The data output from RNA sequencing is a list of all the reads generated, and their sequence [74,70]. This data undergoes quality assessment [75]. For RNA sequencing, sequencing reads are then aligned to the reference genome using a splice-aware sequence alignment algorithm [76].

Alignments can then be visualised using any genome browser or sequence viewing software. RNA stable regions are identified by viewing sequencing read alignments along the RNA of interest. Regions along the RNA sequence where there are more reads aligned (high read coverage) are deemed to be stable regions.

### Stable Regions

A stable region of an RNA sequence according to the invention is a region within any given RNA sequence that RNA sequencing data shows produces more aligned sequencing reads than at least one other region with the same RNA sequence.

### PCR-Based Methods

PCR-based methods are particularly preferred for detection of RNA sequence in the method of the invention.

General PCR approaches are well known to those skilled in the art [77]. Various other developments of the basic PCR approach may also be advantageously applied to the method of the invention. Examples are discussed briefly below.

### Multiplex-PCR

Multiplex-PCR utilises multiple primer sets within a single PCR reaction to produce amplified products (amplicons) of varying sizes that are specific to different target RNA, cDNA or DNA sequences. By targeting multiple sequences at once, diagnostic information may be gained from a single reaction that otherwise would require several times the reagents and more time to perform. Annealing temperatures and primer sets are generally optimized to work within a single reaction, and produce different amplicon sizes. That is, the amplicons should form distinct bands when visualized by gel or capillary electrophoresis. Multiplex PCR can be used in the method of the invention to distinguish the type of sample it is applied to in a single sample or reaction.

### MLPA

Multiplex ligation-dependent probe amplification (MLPA) (U.S. Pat. No. 6,955,901) is a variation of the multiplex polymerase chain reaction that permits multiple targets to be amplified with only a single primer pair. Each probe consists of two oligonucleotides which recognize adjacent target sites on the DNA. One probe oligonucleotide contains the sequence recognized by the forward primer, the other the sequence recognized by the reverse primer. Only when both probe oligonucleotides are hybridized to their respective targets, can they be ligated into a complete probe. The advantage of splitting the probe into two parts is that only the ligated oligonucleotides, but not the unbound probe oligonucleotides, are amplified. If the probes were not split in this way, the primer sequences at either end would cause the probes to be amplified regardless of their hybridization to the template DNA. Each complete probe has a unique length, so that its resulting amplicons can be separated and identified (for example by capillary electrophoresis among other methods). Since the forward primer used for probe amplification is fluorescently labeled, each amplicon generates a fluorescent peak which can be detected by a capillary sequencer. Comparing the peak pattern obtained on a given sample with that obtained on various reference samples measures presence or absence (or the relative quantity) of each amplicon. This then indicates presence or absence (or the relative quantity) of the target sequence present in the sample DNA. The products can also be detected using gel electrophoresis or microfluidic systems such as Shimadzu MultiNA. The use of reference samples to establish presence or absence is the same. More information about MLPA is available on the World Wide Web at http://www.mlpa.com. MLPA probes may be synthesized as oligonucleotides, by methods known to those skilled in the art. MLPA probes and reagents may be commercially produced by and purchased from HRC-Holland (http://www.mlpa.com).

### Quantitative PCR

Quantitative PCR (Q-PCR) is used to measure the quantity of a PCR product (commonly in real-time). Q-PCR quantitatively measures starting amounts of DNA, cDNA, or RNA. Q-PCR is commonly used to determine whether a DNA sequence is present in a sample and the number of its copies in the sample. Quantitative real-time PCR has a very high degree of precision. Q-PCR methods use fluorescent dyes, such as SYBR Green, EvaGreen or fluorophore-containing DNA probes, such as TaqMan, to measure the amount of amplified product in real time. Q-PCR is sometimes abbreviated to RT-PCR (Real Time PCR) or RQ-PCR. QRT-PCR or RTQ-PCR.

### Primers

The term “primer” refers to a short polynucleotide, usually having a free 3′OH group, that is hybridized to a template and used for priming polymerization of a polynucleotide complementary to the template. Such a primer is preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20 nucleotides in length.

In conventional primer design for amplifying RNA marker sequences, primers are typically designed to cover exon boundaries, to prevent amplification of genomic DNA.

The invention relates to targeting stable regions of RNA transcripts, which is particularly useful when amplifying markers from degraded samples. As will be readily apparent, once a stable region is identified, that region can be used to type samples containing RNA having RIN values from 8 to 10 as well as below 8. Both options thus form part of the present invention.

In one embodiment the primer of the invention for use in a method of the invention does not span an exon boundary.

Although not preferred, in one embodiment the primer of the invention for use in a method of the invention may span an exon boundary.

### Labelling of Primers

Methods for labelling primers are well known to those skilled in the art, and include:

Primers can be labelled enzymatically [78] or chemically (including automated solid-phase chemical synthesis; [79]).

Primers can be labelled with; a fluorescence label (fluorophore; [80]), biotin [81], or radioactive and non-radioactive labels (for example digoxigenin) [82].

Primers labelled by such methods form part of the invention.

### Probe-Based Methods

Probe-based methods may be applied to detect the RNA sequences in the method of the invention. Methods for hybridizing probes to target nucleic acid sequences are well known to those skilled in the art [83].

Probe-based methods include in situ hybridization.

The term “probe” refers to a short polynucleotide that is used to detect a polynucleotide sequence that is at least partially complementary to the probe, in a hybridization-based assay. The probe may consist of a “fragment” of a polynucleotide as defined herein. Preferably such a probe is at least 10, more preferably at least 20, more preferably at least 30, more preferably at least 40, more preferably at least 50, more preferably at least 100, more preferably at least 200, more preferably at least 300, more preferably at least 400 and most preferably at least 500 nucleotides in length.

### Labelling of Probes

Methods for labelling probes are well known to those skilled in the art, and include:

Probes can be labelled enzymatically [83,78] or chemically (including automated solid-phase chemical synthesis) [79].

Probes can be:

Molecular Beacon [84], TaqMan [80], Scorpion [85], In situ hybridization probes [86], Radioactive and non-radioactive [87,82].

Probes labelled by such methods form part of the invention.

### Polynucleotides

The term “polynucleotide(s),” as used herein, means a single or double-stranded deoxyribonucleotide or ribonucleotide polymer of any length but preferably at least 5 nucleotides, and include as non-limiting examples, coding and non-coding sequences of a gene, sense and anti-sense sequences complements, exons, introns, genomic DNA, cDNA, pre-mRNA, mRNA, rRNA, siRNA, miRNA, tRNA, naturally occurring DNA or RNA sequences, synthetic RNA and DNA sequences, and fragments thereof. In one embodiment the nucleic acid is isolated, that is separated from its normal cellular environment. The term “nucleic acid” can be used interchangeably with “polynucleotide”.

### Methods for Extracting Nucleic Acids

Methods for extracting nucleic acids are well-known to those skilled in the art [83].

Specialized extraction procedures can optionally be applied depending on the sample type, as discussed in the example section. For example, RNA from forensic type samples can be extracted using a DNA-RNA co-extraction method, as described by Bowden et al. 2011 [88].

All such methods are intended to be included within the scope of the present invention.

### Percent Identity

Variant polynucleotide sequences preferably exhibit at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a specified polynucleotide sequence. Identity is found over a comparison window of at least 10 nucleotide positions, more preferably at least 11 nucleotide positions, more preferably at least 12 nucleotide positions, more preferably at least 13 nucleotide positions, more preferably at least 14 nucleotide positions, more preferably at least 15 nucleotide positions, more preferably at least 16 nucleotide positions, more preferably at least 17 nucleotide positions, more preferably at least 18 nucleotide positions, more preferably at least 19 nucleotide positions, more preferably at least 20 nucleotide positions, more preferably at least 21 nucleotide positions and most preferably over the entire length of the specified polynucleotide sequence. The invention includes such variants.

Polynucleotide sequence identity can be determined in the following manner. The subject polynucleotide sequence is compared to a candidate polynucleotide sequence using BLASTN (from the BLAST suite of programs, version 2.2.5 [November 2002]) in bl2seq [89], which is publicly available from NCBI (ftp://ftp.ncbi.nih.gov/blast/). The default parameters of bl2seq are utilized except that filtering of low complexity parts should be turned off.

The identity of polynucleotide sequences may be examined using the following unix command line parameters:
bl2seq -i nucleotideseq1 -j nucleotideseq2 -F -p blastn
   The parameter -F turns off filtering of low complexity sections. The parameter -p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman-Wunsch; [90]). A full implementation of the Needleman-Wunsch global alignment algorithm is found in the needle program in the EMBOSS package [91] which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program, which computes an optimal global alignment of two sequences without penalizing terminal gaps, may be used to calculate sequence identity [92].

Sequence identity may also be calculated by aligning sequences to be compared using Vector NTI version 9.0, which uses a Clustal W algorithm [93], then calculating the percentage sequence identity between the aligned sequences using Vector NTI version 9.0 (Sep. 2, 2003 ©1994-2003 InforMax, licensed to Invitrogen).

In general terms therefore the invention provides a method for the detection of an RNA sequence in a sample. The method including the steps of:

a) providing a sample, and

b) detecting the RNA sequence using at least one primer or probe complementary to a stable region of the RNA sequence.

The stable region of the RNA sequence will preferably be identified using RNA sequencing of the sample and, in particular, will be identified as a region in the RNA sequence which has more aligned sequencing reads than another region, or regions, of the same RNA sequence.

Stable regions have been identified and discussed herein and stable regions for use in the methods of the invention can be selected from the group comprising SEQ ID NO:1 to SEQ ID NO:19 or a complement of any one thereof.

Primers have also been identified and discussed herein and primers can be selected from the group comprising SEQ ID NO:20 to SEQ ID NO:57 or complement of any one thereof.

Additionally, in a more specific sense, the invention can be seen to include a nucleotide sequence comprising at least 5 nucleotides with at least 70% identity to a sequence selected from SEQ ID NO:1 to SEQ ID NO:19 or a complement thereof.

Further, and again in a more specific sense, the invention can be seen to include a nucleotide sequence comprising at least 5 nucleotides of a sequence selected from SEQ ID NO:1 to SEQ ID NO:19 or a complement thereof.

Further, and again in a more specific sense, the invention can be seen to include a nucleotide sequence comprising at least 10 nucleotides with at least 70% identity to a sequence selected from SEQ ID NO:1 to SEQ ID NO:19 or a complement thereof.

Further, and again in a more specific sense, the invention can be seen to include a nucleotide sequence comprising at least 10 nucleotides of a sequence selected from SEQ ID NO:1 to SEQ ID NO:19 or a complement thereof.

Further, and again in a more specific sense, the invention can be seen to include a nucleotide sequence selected from any one of SEQ ID NO:20 to SEQ ID NO:57.

The use of a nucleotide sequence as is defined above in the typing of a sample including RNA specifically forms part of the present invention.

As will be apparent, samples containing RNA can be taken from a variety of sources. The most preferable sample is a biological tissue sample which can be either solid or liquid.

The method of the present invention is particularly suitable for use in the forensic field and therefore the sample can be a forensic sample of any type containing RNA such as selected from the group comprising blood, semen (with or without spermatozoa), saliva, vaginal material and menstrual fluid.

The RNA should preferably be extracted from the sample prior to the detecting step and the RNA sequence can be detected directly or indirectly as will be known to a skilled person. It is however preferred that the RNA sequence is detected indirectly by detection of a complementary DNA (cDNA) corresponding to the RNA sequence.

The invention, in a more particular sense, can also be seen to include a method of typing a sample including RNA where the method includes the steps of:

a) providing a sample including RNA;

b) detecting one or more RNA sequences in the sample using at least one primer or probe complementary to the one or more stable region of the RNA; wherein the stable RNA sequence is specific for the type of sample; and wherein detecting the stable RNA sequence indicates the type of sample.

The invention, in another sense, can be seen to include a method of typing a sample including degraded RNA, the method including the steps:

a) providing a sample including degraded RNA;

b) detecting one or more stable RNA sequences in the sample using at least one primer or probe complementary to the one or more stable region of the degraded RNA;

wherein the stable RNA sequence is specific for the type of sample; and
wherein detecting the target RNA sequence indicates the type of sample.

In another embodiment the invention can be a method for the identification of a stable region in RNA in a sample, the method comprising:

a) providing a sample including RNA,

b) isolating total RNA from the sample,

c) removing DNA from the sample

d) generating cDNA complementary to the RNA in the sample,

e) sequencing the cDNA,

wherein the stable region of the RNA sequence is identified as a region in the RNA sequence which has more aligned sequencing reads than another region, or regions, of the same RNA sequence.

As has been previously discussed, the method can be applied to RNA which has degraded to a condition which had previously been thought not to be useful as a means for typing/identifying the source of the sample from which it has been extracted. The methods of the invention can be used to type/identify the source of samples in which the RNA content has a RIN value of less than 8. As stable regions in RNA having a value of less than eight will also be present in RNA having a RIN value of between 8 and 10, once the stable regions have been identified those stable regions can also be used to identify/type the source of the sample having an RIN of between 8 and 10. Therefore, the method can be used to type/identify the source of samples having any RIN value, including samples in which the RIN value cannot be determined.

As has been discussed previously, the stable region of the RNA sequence can be identified as a region in the RNA sequence which has more aligned sequencing reads than another region, or regions, of the same RNA sequence.

As will be readily apparent to a skilled person, the RNA sequence will preferably be detected using a primer or a probe. As will also be apparent, the RNA sequence can be detected using more than one primer or probe (e.g. two primers) if appropriate/desired.

The primers and/or probes should preferably correspond to, or be complementary to, or be capable of hybridising to, a sequence within the stable region of the RNA that has been extracted from the sample. The primers are used to amplify the part of the stable region bound by the primers, such as by a polymerase chain reaction (PCR) method. The PCR method can be selected from standard PCR, reverse transcriptase PCT (RT-PCR) and quantitative reverse transcriptase PCR (qRT-PCR).

In addition, and as will also be readily apparent to a skilled person, the RNA sequence can be detected using a probe. This will preferably correspond to, or be complementary to, a sequence within the stable region of the RNA that has been extracted from the sample.

The RNA sequence can be encoded by a marker gene specific for the type of sample. That is, the expression of the RNA sequence, or presence of the RNA sequence, in the sample, is diagnostic for the type of sample. For example, when the sample is circulatory blood, the marker gene is selected from:
Hemoglobin delta (HBD), and/or
Solute carrier family 4 (anion exchanger), member 1 (Diego blood group) (SLC4A1)
Glycoprotein A (GYPA).
   When the sample contains Saliva, the marker gene is selected from:
Follicular Dendritic Cell Secreted Protein (FDCSP), and/or
Histatin 3 (HTN3)
Statherin (STATH).
   When the sample contains spermatozoa, the marker gene is selected from:
Protamine 1 (PRM1), and/or
Transition protein 1 (during histone to protamine replacement) (TNP1) and/or
Protamine 2 (PRM2).
   When the sample is seminal fluid, the marker gene is selected from:
Kallikrein-related peptidase 2 (KLK2), and/or
Microseminoprotein Beta (MSMB) and/or
Transglutaminase 4 (TGM4).
   When the sample is menstrual fluid, the marker gene is selected from:
Matrix metallopeptidase 10 (MMP10), and/or
Stanniocalcin 1 (STC1), and/or
Matrix metallopeptidase 3 (MMP3)
Matrix metallopeptidase 11 (MMP11).
   When the sample is vaginal material, the marker gene is selected from:
Cytochrome P450 Family 2 Subfamily B Member 7 (CYP2B7P) and/or
Lactobacillus crispatus protein (L.gass) and/or
Lactobacillus gasseri protein (L.crisp).

The detection process of the present invention can involve the use of either a primer or a probe capable of hybridising to the stable region of the RNA sequence, or a cDNA corresponding to the stable region or a complement thereof. The method may involve using just one pair of primers, or a single probe, to type the sample. Alternatively multiple pairs of primers, or multiple probes, may be used.

The primer or the probe can include (i) a sequence of at least 5 nucleotides with at least 70% identity to any part of the sequence of any one of SEQ ID NO:1 to 19 or a complement thereof or (ii) a sequence of at least 5 nucleotides with at least 70% identity to the sequence of any one of SEQ ID NO:1 to 19, or a complement thereof or (iii) a sequence of at least 5 nucleotides of the sequence of any one of SEQ ID NO:1 to 19, or a complement thereof or (iv) a sequence of at least 5 nucleotides of the sequence of any one of SEQ ID NO:1 to 19, or a complement thereof or (v) a sequence selected from any one of SEQ ID NO:20 to 57 or (vi) a label or tag attached to a sequence selected from any one of those sequences.

The primer or the probe can include (i) a sequence of at least 10 nucleotides with at least 70% identity to any part of the sequence of any one of SEQ ID NO:1 to 19 or a complement thereof or (ii) a sequence of at least 10 nucleotides with at least 70% identity to the sequence of any one of SEQ ID NO:1 to 19, or a complement thereof or (iii) a sequence of at least 10 nucleotides of the sequence of any one of SEQ ID NO:1 to 19, or a complement thereof or (iv) a sequence of at least 10 nucleotides of the sequence of any one of SEQ ID NO:1 to 19, or a complement thereof or (v) a sequence selected from any one of SEQ ID NO:20 to 57 or (vi) a label or tag attached to a sequence selected from any one of those sequences.

By way of example, typing of a sample can be undertaken using multiplex PCR performed with multiple primers, at least one of which is diagnostic for the type of sample.

Preferably multiplex PCR is performed using at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30, more preferably at least 31, more preferably at least 32, more preferably at least 33, more preferably at least 34, more preferably at least 35, more preferably at least 36, more preferably at least 37, more preferably at least 38 primers of the invention.

The invention also allows the provision of a kit that includes at least one primer or probe according to the present invention. Such a kit can include any number of primers or probes and in particular the kit can include at least 2, more preferably at least 3, more preferably at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30, more preferably at least 31, more preferably at least 32, more preferably at least 33, more preferably at least 34, more preferably at least 35, more preferably at least 36, more preferably at least 37, more preferably at least 38 primers or probes of the invention. Combinations of primers and probes may also be provided in such kits.

As will be readily apparent, the kit should also include instructions for use, if such instructions are needed.

The invention also allows the provision of microarrays or chips or like products that include sequences that have been identified herein as stable areas of RNA that can be used to type/identify samples or that are complementary thereto. These sequences have been used to generate primers and probes that can be used on microarrays or chips or like products for the detection of nucleotide sequences.

Such microarrays or chips are of particular commercial importance as they allow the efficient and accurate identification of unknown samples including RNA, including where the RNA has been degraded. The creation of such products is well within the abilities of the person skilled in the art once they have the benefit of knowledge of the present invention.

### BRIEF DESCRIPTION OF DRAWINGS

FIG. 1. Expression patterns of HBD, SLC4A1, TNP1, KLK2, MMP3 and STC1. Amplification of six samples per body fluid; BL=circulatory blood, SA=saliva/buccal, SM=semen (with spermatozoa), SF=seminal fluid (without spermatozoa), MF=menstrual fluid, VM=vaginal material. The same samples and donors were not necessarily used for the assessment of all markers. Only TNP1 and KLK2 were amplified from seminal fluid samples.

FIG. 2. Sensitivity comparison of the six novel mRNAs to four well-known markers [1]. Top: HBD and SLC4A1 compared to GYPA using three samples each of 2, 1 and 0.5 μL circulatory blood and a primer concentration of 0.2 μM. Second from top: TNP1 compared to PRM2 using 9 samples of 1 μL semen from three donors and a primer concentration of 0.05 μM. Second from bottom: KLK2 compared to TGM4 using three samples each of 2, 1 and 0.5 μL seminal fluid (azoospermic) and a primer concentration of 0.1 μM. Bottom: MMP3 and STC1 compared to MMP11 using nine menstrual fluid samples (days 2 and 3) from two donors and a primer concentration of 0.1 μM. Average peak heights (APH) and standard deviations were calculated from three technical replicates.

FIG. 3. RNA-Seq results (fragments per kilobase of exon per million fragments mapped, FPKM) for two known markers (GYPA, MMP11) and four novel mRNA candidates (HBD, SLC4A1, MMP3, STC1). BL=circulatory blood; BU=buccal; MF=menstrual fluid; VM=vaginal material.

FIG. 4. Primer sequences and expected amplicon sizes of all markers included in the three multiplex assays.

FIG. 5. Body fluid specificity of the three multiplex assays.

FIG. 6. Electropherograms of A. a buccal sample, B. a menstrual fluid sample, and C. a mixed sample of semen and vaginal material. Each sample was amplified using multiplex D (top), multiplex Q (middle), and multiplex P (bottom).

FIG. 7. The effect of multiplexing. APH obtained in multiplex (white bars) and uniplex reactions (shaded) for A. 0.05 μM FDCSP and 0.012 μM HTN3, B. 0.05 μM HBD and 0.04 μM SLC4A1, C. 0.04 μM MMP10 and 0.02 μM STC1, D. 0.03 μM PRM1 and 0.04 μM TNP1, E. 0.14 μM KLK2 and 0.03 μM MSMB, and F. 0.02 μM CYP2B7P.

FIG. 8. Resolution of body fluid mixtures. Values are given in RFU. MF was collected on day 2 of the uterine cycle from a naturally cycling donor. Samples were 14 weeks old when further components were added. VM was collected on day 19 of the uterine cycle from a naturally cycling donor. Samples were 11 weeks old when further components were added. For samples containing MF, VM, or semen as component 1, the RNA was diluted 1:75, 1:50, and 1:8, respectively, prior to RT. Further dilution of cDNA samples was carried out for MF-blood, MF-semen (5 μL and 10 μL), and semen-saliva mixtures to adjust peak heights. SA=saliva, SM=semen.

FIG. 9. Amplification of post-coital vaginal samples using multiplex P.

FIG. 10. Marker detection in aged samples. Peak heights (RFU) were obtained from aged body fluid samples, aged RNA, and aged cDNA, stored at room temperature or frozen for 15 to 35 months.

FIG. 11. Analysis of case-type samples. Expected results are highlighted.

¹Expected results were disclosed after completion of mRNA analysis. BL=circulatory blood, SA=saliva, SP=spermatozoa, SF=seminal fluid, VM=vaginal material, NR=no result.
²CellTyper amplifications were performed as published [2]. PCR products were separated on a Genetic Analyzer 3130xl, with a peak amplitude threshold of 100 RFU.

The invention will now be exemplified by way of the following non-limiting examples.

### EXAMPLE 1: IDENTIFICATION OF RNA STABLE REGIONS IN BODY SAMPLES

### Materials and Methods

### Identification of Body Fluid-Specific Candidate Genes

Candidate mRNAs for the identification of circulatory blood (HBD, SLC4A1) and menstrual fluid (MMP3, STC1) were selected from RNA-Seq data of degraded body fluids as published previously [22]. Semen marker candidates (TNP1, KLK2) were chosen from gene expression databases (TiGER, PaGenBase) [24,25] with respect to their physiological function in the body.

### Primer Design

Primers for HBD, SLC4A1, MMP3 and STC1 were designed to target transcript stable regions (StaRs) as described previously [23] using the OligoAnalyzer 3.1 online tool (Integrated DNA Technologies, Inc., Coralville, Iowa, USA). Sequencing coverage maps were viewed using the Geneious v.5.6.7 software (Biomatters Ltd., Auckland, New Zealand) and regions of high coverage selected for primer design. Primers for TNP1 and KLK2 were designed using conventional primer design strategy. The specificity of all primers to their intended mRNA targets was verified using Primer-BLAST [26]. Primer sequences and expected amplicon sizes are listed in Table 2.

TABLE 2
Primer sequences and expected amplicon sizes of the novel body fluid
markers.

| Target body | | Accession | | Product size |
|---|---|---|---|---|
| fluid | Marker | number | Primer Sequence (5′-3′) | (bp) |
| Circulatory | Haemoglobin | NM_000519.3 | F: ACTGCTGTCAATGCCCTGTG | 176 |
| blood | delta (HBD) | | R: ACCTTCTTGCCATGAGCCTT | |
| | Solute carrier | NM_000342.3 | F: AACTGGACACTCAGGACCAC | 102 |
| | family 4 (anion | | R: GGATGTCTGGGTCTTCATATTCCT | |
| | exchanger), | | | |
| | member 1 | | | |
| | (Diego blood | | | |
| | group) (SLC4A1) | | | |
| Semen | Transition | NM_003284.3 | F: GATGACGCCAATCGCAATTACC | 102 |
| containing | protein 1 (during | | R: CCTTCTGCTGTTCTTGTTGCTG | |
| spermatozoa | histone to | | | |
| | protamine | | | |
| | replacement) | | | |
| | (TNP1) | | | |
| Seminal | Kallikrein-related | NM_005551.4 | F: CAGTCATGGATGGGCACACT | 141 |
| fluid | peptidase 2 | | R: ACCCTCTGGCCTGTGTCTTC | |
| | (KLK2) | | | |
| Menstrual | Matrix | NM_002422.3 | F: CCATGCCTATGCCCCTG | 84 |
| fluid | metallopeptidase | | R: GTCCCTGTTGTATCCTTTGTCC | |
| | 3 (MMP3) | | | |
| | Stanniocalcin 1 | NM_003155.2 | F: TGCCCAATCACTTCTCCAACAG | 103 |
| | (STC1) | | R: TTCTCCATCAGGCTGTCTCTG | |

### Collection of Body Fluid Samples

Six samples each of 50 μL circulatory blood, semen and seminal fluid (azoospermic), as well as saliva/buccal mucosa, menstrual and non-menstrual vaginal swabs were obtained from healthy, consenting volunteers, as approved by the University of Auckland Human Participants Ethics Committee (UAHPEC). Blood was drawn using a sterile AKKU-CHEK® Safe-T-Pro Plus lancet (Roche Diagnostics USA, Indianapolis, Ind., USA). Blood, semen and seminal fluid aliquots were deposited onto sterile Cultiplast® rayon swabs. Buccal, menstrual and vaginal samples were obtained by volunteers themselves using sterile swabs. All samples were allowed to dry overnight at ambient laboratory conditions and then extracted as described below.

### RNA Extraction and Purification

Total RNA from body fluid samples was prepared as described previously [22,23] using the Promega® DNA IQ and ReliaPrep™ RNA Cell Miniprep Systems (Promega Corporation, Madison, Wis., USA) following the manufacturer's instructions. Genomic DNA was removed by incorporating an on-column DNase I treatment during the RNA extraction process. RNA was eluted in 45 μL nuclease-free water. The absence of genomic DNA was verified by real-time PCR using the Quantifiler® Human DNA quantification kit (Life Technologies™ by Thermo Fisher Scientific, Inc., Waltham, Mass., USA) with 1 μL purified RNA in a 12.5 μL reaction. Samples which contained residual DNA were treated with TURBO™ DNase (Invitrogen™ by Thermo Fisher Scientific, Inc.) and re-quantified until no DNA was detectable.

cDNA Synthesis

Complementary DNA (cDNA) was prepared using the High Capacity cDNA Reverse Transcription Kit (Applied Biosystems™ by Thermo Fisher Scientific, Inc.) according to the manufacturer's instructions. Ten microlitres of DNA-free RNA were subjected to reverse transcription in a 20 μL reaction. Synthesis was performed on a GeneAmp PCR System 9700 thermal cycler (Applied Biosystems™ by Thermo Fisher Scientific, Inc.) using the following program: 25° C. for 10 min, 37° C. for 120 min, followed by 85° C. for 5 min and hold at 4° C.

### Polymerase Chain Reaction (PCR)

### PCR Reactions

Body fluid cDNA samples were amplified using the QIAGEN® Multiplex PCR Kit (Qiagen GmbH, Hilden, Germany) according to the manufacturer's instructions. Two microlitres of cDNA were amplified in 25 μL PCR reactions containing 12.5 μL of 2× PCR master mix. Primer concentrations for specificity testing were as follows: 0.05 μM (HBD), 0.03 μM (SLC4A1), 0.08 μM (TNP1), 0.4 μM (KLK2), 0.02 μM (MMP3), 0.02 μM (STC1). Primer concentrations for comparison were 0.2 μM (circulatory blood), 0.05 μM (semen), and 0.1 μM (seminal and menstrual fluid), respectively. Finally, nuclease-free water was added to achieve a total volume of 25 μL for each reaction.

### PCR Cycling Conditions

PCR cycling conditions for amplification on the GeneAmp PCR System 9700 were as published previously [22,23,1]: initial denaturation at 95° C. for 15 min, followed by 35 cycles of 94° C. for 30 s, 58° C. for 3 min and 72° C. for 1 min, final elongation at 72° C. for 45 min and cooling down to 4° C.

### Capillary Electrophoresis and Data Analysis

PCR products were separated on a Genetic Analyzer 3130xl (Applied Biosystems™ by Thermo Fisher Scientific, Inc.). One microliter of amplified PCR product was mixed with 9 μL of a formamide/size standard stock solution, created by adding 15 μL GeneScan™ 500 ROX™ to 1000 μL HiDi™ formamide. Results were analysed with GeneMapper v.3.2.1 (Applied Biosystems™ by Thermo Fisher Scientific, Inc.) using a peak amplitude threshold of 50 RFU.

### Results and Discussion

### Selection of Body Fluid Marker Candidates

Whole transcriptome paired-end sequencing (2×100 bp) of circulatory blood (2 donors) and menstrual fluid (1 donor) was performed in order to identify highly expressed biomarkers possibly exclusive to each body fluid type [22]. Processed and merged sequencing reads for each sample were aligned to the human reference sequence assembly hg19 (GRCh37) to allow for the determination of the maximum count values for each detected transcript [22]. Data were sorted by maximum count numbers and compared between sample types to exclude concomitantly expressed genes and identify highly abundant and possibly specific body fluid markers. Four mRNA candidates were identified from this data set: haemoglobin delta (HBD) and solute carrier family 4, member 1 (SLC4A1) for circulatory blood, as well as matrix metallopeptidase 3 (MMP3) and stanniocalcin 1 (STC1) for menstrual fluid.

Two further candidate genes were selected from two gene expression databases (TiGER, PaGenBase) [24,25] based on their putative physiological function in the human body: transition protein 1 (TNP1) for spermatozoa and kallikrein-related peptidase 2 (KLK2) for seminal fluid which may be free of spermatozoa.

### RNA-Seq Data Analysis

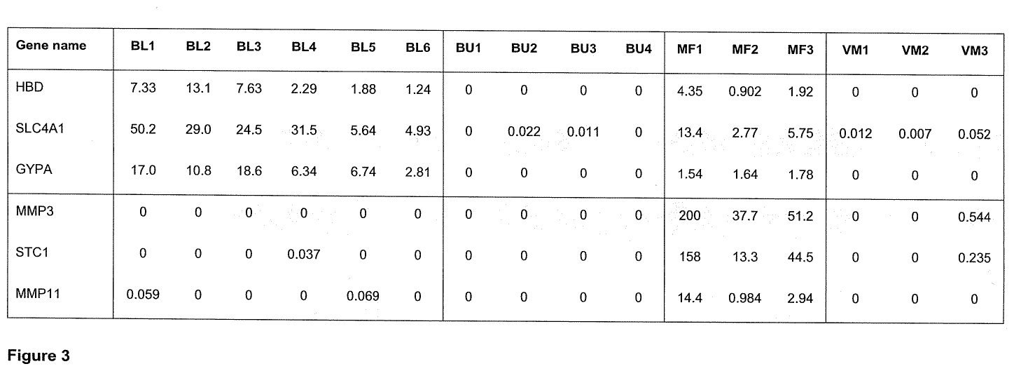

FIG. 3 shows that no HBD and GYPA fragments were sequenced in buccal and vaginal material samples, whereas SLC4A1 was detected in two and three samples, respectively (FPKM<0.06). The highest FPKM values in both circulatory blood and menstrual fluid were observed for SLC4A1, except in sample BL5, which showed higher levels of GYPA. HBD was detected at relatively low levels; however, FPKM values were higher than GYPA in two menstrual fluid samples and no fragments were detected in buccal or vaginal samples.

All menstrual fluid marker candidates were undetected in buccal mucosa (FIG. 3). MMP3 was also undetectable in circulatory blood, whereas STC1 was sequenced in one and MMP11 in two samples (FPKM<0.07). In addition, one vaginal material sample (VM3) contained low levels of MMP3 and STC1 (FPKM<0.6). In menstrual fluid, FPKM values for MMP3 and STC1 were up to 38.3-fold and 15.1-fold higher than MMP11, respectively.

### Specificity Screening

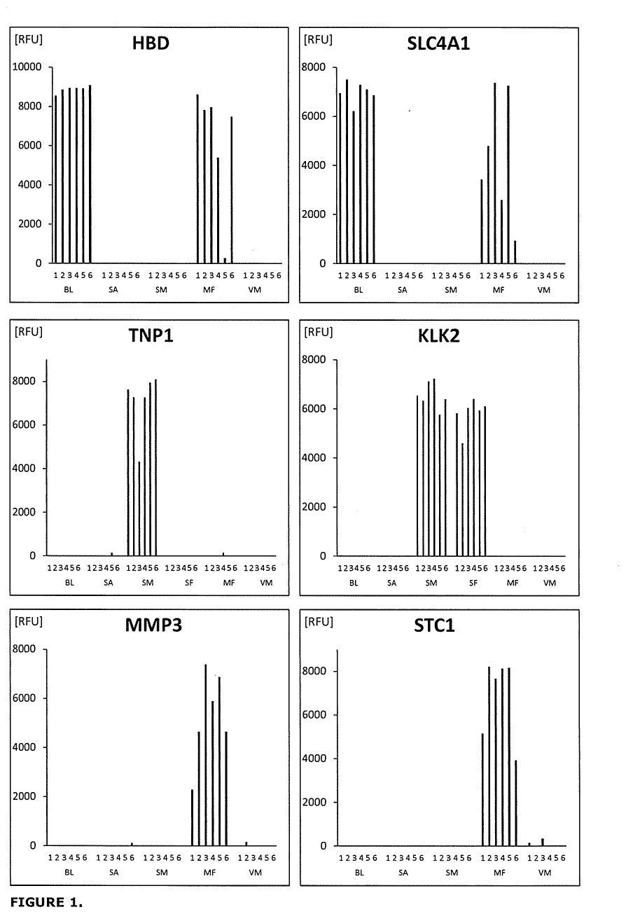

The expression profiles of the six body fluid marker candidates were evaluated by singleplex endpoint RT-PCR. Six samples per body fluid (50 μL circulatory blood and semen, whole buccal, menstrual and non-menstrual vaginal swabs) from various donors were amplified using 2 μL of cDNA synthesised from total RNA. When cross-reactive peaks were observed (TNP1, MMP3 and STC1, FIG. 1), the corresponding samples were reamplified to verify signal reproducibility. Reverse transcription negative (RT−) controls omitting the RT enzyme were also prepared for each sample and amplified. All RT− controls were negative (data not shown).

### Haemoglobin Delta (HBD)

The haemoglobin delta or δ-globin gene is part of the human β-globin gene cluster located on chromosome 11p15.5. Together with two alpha chains, two delta chains constitute the HbA₂ tetramer (α₂δ2), which comprises about 2-3% of the total haemoglobin in adult humans [27]. The coding region of HBD has strong sequence homology with HBB, both of which are expressed in bone marrow and reticulocytes [27,28]. Mutations in the HBD gene can result in clinically insignificant δ-thalassaemia, characterised by a reduced ability of the body to produce HbA₂ [27].

HBD mRNA was exclusively present in circulatory blood and menstrual fluid (FIG. 1). All circulatory blood and five of six menstrual fluid samples produced signals above 5000 RFU. The remaining menstrual sample (MF 5) produced a signal of 272 RFU, likely due to a lower blood content as this sample was collected on day 4 of the menstrual cycle and the donor reported only light bleeding. Accordingly, the obtained swab was lighter red in colour than the day 2 or 3 samples. All semen, buccal, and vaginal material samples were negative (FIG. 1). These results demonstrate high abundance of HBD in blood and a specific expression pattern despite high sample input volumes.

Although HBD expression is known to reach only about 50% of that of HBB [27], our data show consistent and efficient detection of HBD mRNA and therefore demonstrate suitability of this marker for the identification of blood. The reduced expression of HBD is also advantageous given that the relatively strong and ubiquitous expression of HBB can lead to amplification from non-target body fluids [3,10]. While some of those observed signals may have been due to the presence of trace amounts of blood in a sample rather than true HBB expression, such findings clearly complicate the interpretation of results. Since HBD shows the same expression pattern as HBB, its reduced transcription rate is beneficial in this context as it increases marker specificity (FIG. 1).

### Solute Carrier Family 4 (Anion Exchanger), Member 1 (Diego Blood Group) (SLC4A1)

SLC4A1, also known as anion exchanger 1 (AE1) or band 3, is located on chromosome 17q21-22, and is the main integral protein in the erythrocyte membrane, connecting the lipid bilayer to the protein network through interactions with ankyrin-1 and proteins 4.1 and 4.2 [29]. SLC4A1 also interacts with glycophorin A (GYPA) and haemoglobin [30]. The C-terminal domain functions as an anion exchanger, increasing the overall capacity of blood to transport CO₂ [29,30]. Numerous mutations in the SLC4A1 gene have been discovered, leading to conditions such as hereditary spherocytosis, southeast Asian ovalocytosis and hereditary acanthocytosis, all of which affect erythrocyte phenotype and result in minor to severe anaemia [29,30].

FIG. 1 shows that, at the primer concentration of 0.03 μM, SLC4A1 was specific to samples containing blood and was not present in semen, buccal or vaginal material samples. SLC4A1 mRNA was detected in all circulatory blood samples and two of six menstrual fluid samples at peak heights above 6000 RFU. The remaining menstrual fluid samples produced peaks of 3430 RFU (MF 1), 4804 RFU (MF 2), 2596 RFU (MF 4) and 937 RFU (MF 6), respectively. This may indicate slightly reduced expression of SLC4A1 in comparison to HBD, which on average produced 1.4-fold higher RFU from menstrual samples, however the difference was not statistically significant (Student's t-test, p>0.1). Furthermore, the primer concentration used for SLC4A1 (0.03 μM) was lower than that of HBD (0.05 μM) and different samples were used for the evaluation of both markers. Importantly, SLC4A1 was specific to samples containing blood and was not present in semen, buccal or vaginal material samples (FIG. 1).

### Transition Protein 1 (During Histone to Protamine Replacement) (TNP1)

TNP1 has been mapped to chromosome 2q35-q36. Together with the larger TNP2, TNP1 replaces histones in the nuclei of elongating and condensing spermatids during spermiogenesis and is subsequently replaced by protamines [31]. TNP1 can destabilise nucleosomes and prevent DNA bending, and in turn promotes the repair of strand breaks by serving as an alignment factor [31]. Mutations in the promoter region of the TNP1 gene were found to reduce TNP1 expression and may contribute to male infertility [52].

Our results demonstrate strong expression of TNP1 in semen samples containing spermatozoa (FIG. 1). Notably, TNP1 was not detectable in six samples from an azoospermic donor or any of the circulatory blood and vaginal material samples. However, one saliva and one menstrual fluid sample produced peaks (147 and 152 RFU, respectively), although these were easily distinguished from semen samples, all of which exceeded 4300 RFU. The saliva and menstrual fluid samples were reamplified to verify signal reproducibility and no peaks were observed, indicating that the initially observed signals likely resulted from amplification of trace amounts of TNP1 mRNA or non-specific primer binding. In both samples, replicate amplification clearly distinguished between cross-reactions and target mRNA signals.

### Kallikrein-Related Peptidase 2 (KLK2)

The gene encoding kallikrein-related peptidase 2 (KLK2), also referred to as human kallikrein 2, is located on chromosome 19q3.41. KLK2 is a serine protease synthesised by the prostate gland with high sequence identity to prostate-specific antigen (PSA/KLK3) [32]. It activates the zymogen forms of PSA and urokinase into their enzymatically active forms [32]. In addition, KLK2 possesses the ability to cleave semenogelins I and II, as well as fibronectin [33]. The enzymatic activity of KLK2 may be reversibly regulated by zinc ions, which are highest in the prostate and prostatic fluid [32].

As FIG. 1 shows, KLK2 mRNA was present in all semen samples tested, including six samples donated by an azoospermic individual. No cross-reactions with non-target body fluids were observed. All circulatory blood, buccal, menstrual fluid and vaginal material samples were negative (FIG. 1). Although previous studies have reported the presence of KLK2 mRNA in non-prostatic tissues, including salivary glands and endometrium [34], our findings demonstrate specificity of this mRNA to semen samples.

### Matrix Metallopeptidase 3 (MMP3)

Matrix metallopeptidases (MMPs) are a large family of zinc- or calcium-dependent endopeptidases which catabolise a wide range of substrates and thus regulate protein activity [35,36]. They engage in various roles during tissue degradation and remodelling processes, including menstruation [35,36]. Three members of this family, namely MMPs 7, 10 and 11, have been widely used as forensic menstrual fluid markers [1,3,5-7,36].

MMP3, also known as stromelysin-1 (mapped to 11q22.3) is another member of the MMP superfamily which is highly expressed during menstruation (FIG. 1). This enzyme is one of the key regulators of wound healing and scar formation [35]. Studies in mice have shown that defective MMP3 expression can lead to increased wound size, slowed wound healing and impaired scar contraction [35].

Our results identify MMP3 as a suitable menstrual fluid marker. This mRNA was strongly expressed on days 2 and 3 of the menstrual cycle. All six menstrual fluid samples produced peaks greater than 2000 RFU (FIG. 1). In addition, MMP3 mRNA was not detectable in circulatory blood and semen samples (FIG. 1). However, one buccal (113 RFU) and one vaginal material sample (day 19, 159 RFU) also produced peaks. When these samples were reamplified, no signals were observed (data not shown).

In previous research, MMPs 7, 10 and 11 were introduced as markers specific for the detection of menstruum. Since then, multiple studies reported their expression during uterine phases outside of menstruation [36,7,11]. MMPs have also been detected in circulatory blood [10,7,11], saliva, semen and skin [11]. One study even suggested MMP7 as a general vaginal secretion marker [18]. Here we also observed cross-reactions of MMP3 with saliva/buccal mucosa and vaginal material (FIG. 1). However, these signals were not reproducible and we conclude that they resulted from large sample input (i.e. whole swabs), leading to the amplification of trace amounts of MMP3 mRNA, or unspecific primer binding. Despite this, cross-reactive peaks were below 200 RFU (FIG. 1) and therefore clearly distinguishable from menstrual samples. Overall, the specificity of MMP3 to menstrual discharge is equal to or greater than that of MMPs 7, 10 or 11.

### Stanniocalcin 1 (STC1)

Stanniocalcin 1 (STC1) was originally described as a homodimeric glycoprotein in the corpuscles of bony fishes, where it regulates calcium and phosphate homeostasis [37].

In humans, the STC1 gene is located on chromosome 8p21.2, and the protein may also regulate intracellular calcium and/or phosphate levels as an autocrine or paracrine factor and thus contribute to bone formation [37,38]. In contrast to its function in fish, STC1 activity in humans is thought to be local rather than systemic due to its absence from the circulation [38]. Nevertheless, STC1 appears to be a pleiotropic factor, and other proposed functions include involvement in ischemia, angiogenesis, muscle contractility, as well as immune and inflammatory responses [37,38]. These processes are all known to take place in the endometrium before, during and after menstruation.

Our data confirm that STC1 mRNA is undetectable in circulatory blood samples (FIG. 1). In addition, no signals were obtained from buccal or semen samples, which is in agreement with earlier findings that STC1 mRNA is absent from seminal vesicles [38]. In this study STC1 was strongly expressed in menstrual fluid samples (FIG. 1, average peak height 7703 RFU). However, two of six vaginal material (VM) samples also produced peaks (150 and 347 RFU, respectively). Both VM samples were reamplified and no signals were observed (data not shown). Sample VM 1 was obtained on day 8 of the uterine cycle, which is the early post-menstrual phase. Therefore, this signal may be the result of residual trace amounts of STC1 mRNA which were collected during swabbing. Sample VM 3, in contrast, was collected on day 19 of the uterine cycle from a different individual. This donor used a hormonal contraceptive at the time of sample donation, which could have had an effect on STC1 expression. STC1 expression in ovaries has been reported [38] and it appears that cross-reactions are most likely obtained from vaginal samples. Nevertheless, in this study, STC1 mRNA expression was only observed in menstrual fluid and vaginal material samples, even when the primer concentration was raised to 0.4 μM (data not shown). Further research could address whether the menstrual cycle stage during which a sample is obtained or the use of contraceptives influence STC1 expression.

### Comparison to Existing Markers

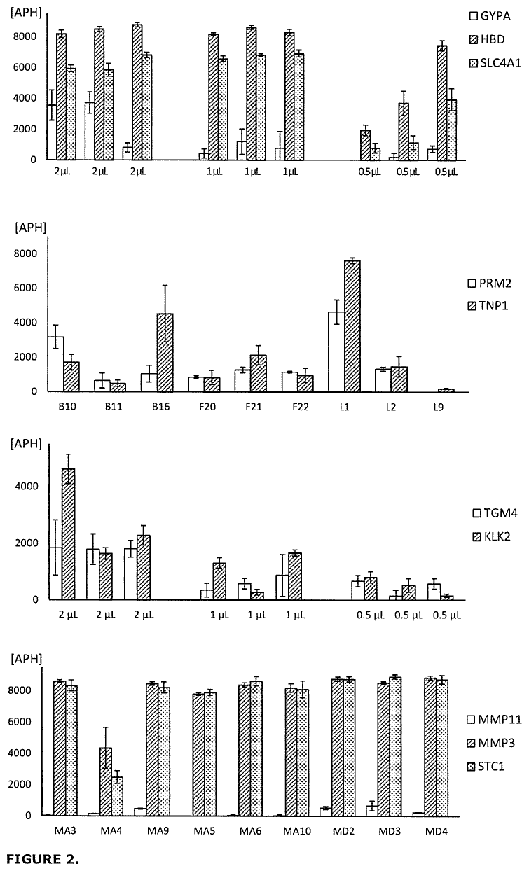

The sensitivity of the six novel body fluid candidates was compared to corresponding well-characterised markers published previously [1] using primer concentrations of 0.2 μM (circulatory blood), 0.05 μM (semen), and 0.1 μM (seminal and menstrual fluid), for comparison, respectively and the same cDNA samples. HBD and SLC4A1 were compared to Glycophorin A (GYPA), TNP1 to protamine 2 (PRM2), KLK2 to transglutaminase 4 (TGM4), and MMP3 and STC1 to MMP11. As FIG. 2 illustrates, all the new mRNAs produced higher average peak heights (APH) from their respective target body fluids than corresponding known markers. Both HBD and SLC4A1 were significantly more sensitive (gave significantly higher signals) for the detection of blood at the primer concentration of 0.2 μM than GYPA (Student's t-test, p<0.0005 for HBD and p<0.005 for SLC4A1). The increased sensitivity of TNP1 from semen samples at a primer concentration of 0.05 μM was also statistically significant (p<0.05). The lowest p-values, however, were obtained for the comparison of MMP11 to MMP3 (p<5·10⁻²¹) and STC1 (p<5·10⁻¹⁷). These findings demonstrate an extremely significant enhancement in detection sensitivity (i.e. signal increase in the same samples) compared to MMP11. Both MMP3 and STC1 mRNAs appear to be much more abundant in the menstruating endometrium than MMP11, while displaying the same expression pattern [1,3,7]. This is also reflected by their respective FPKM values (FIG. 3,7].), although primer design may have contributed to the observed differences in peak height. Only the increase in peak height for KLK2 did not reach statistical significance, although 67% of semen samples produced higher KLK2 signals compared to TGM4.

### Conclusion

This Example evaluated the expression of six new mRNAs for forensic body fluid identification by singleplex endpoint reverse transcription (RT-PCR) and partly using RNA-Seq and have evaluated their expression patterns. All marker candidates were highly abundant in their respective target body fluid type compared to other bodily sources. HBD and SLC4A1 can be used to confirm the presence of circulatory blood. TNP1 mRNA was present in semen which contains spermatozoa, while KLK2 mRNA was exclusive to seminal fluid regardless of spermatozoa presence. MMP3 and STC1 can be used to identify menstrual fluid samples.

All six candidate mRNAs showed increased signal intensity in the same samples compared to corresponding known markers using equal primer concentrations [1]. With the exception of KLK2, the increase in APH reached statistical significance up to an extreme p-value of 5.10⁻²¹ for MMP3 compared to MMP11. Based on RNA-Seq and CE results, both MMP3 and STC1 mRNA appear to be more abundant in the endometrium during menstruation than MMP11 and can therefore facilitate the identification of a blood stain resulting from menses. In particular the detection of STC1 can be useful for discrimination between circulatory blood and menstrual fluid due to its absence from the circulatory system (FIG. 1 [38].

Single cross-reactions were observed for TNP1 with saliva and menstrual fluid, for MMP3 with saliva and vaginal material, and for STC1 with two non-menstrual vaginal samples (FIG. 1). These peaks remained below 350 RFU in all cases and were therefore easily distinguishable from target body fluid signals. In addition, cross-reactions were not reproducible; hence, our data support earlier findings that technical replicates may be useful for mRNA result interpretation [39]. Moreover, it should be kept in mind that the volume of extracted body fluid or RNA/cDNA input amount, respectively, plays a major role in the occurrence of cross-reactive peaks. This study used large body fluid volumes (50 μL or a whole swab) and undiluted cDNA samples in order to uncover trace expression and explore the limits of marker specificity. In view of this, cross-reactions were expected, however all non-target signals were of lower peak height than target signals and were non-reproducible. Additionally, samples in forensic casework are typically of small size, degraded, or otherwise compromised [22,23], thus limiting the amount of RNA and cDNA that can be obtained from a sample. At the primer concentrations used here (FIG. 1), cross-reactions are kept at a minimum, especially when combined with controlled RNA or cDNA input amounts, stringent PCR conditions and suitable interpretation guidelines [8,10,11,13]. Nevertheless, cross-reactions complicate the resolution of body fluid mixtures.

### Summary

The simultaneous assessment of multiple mRNAs per body fluid can help avoid false positives, since it is less likely that all typed markers would falsely indicate the presence of a certain body fluid [9]. The six novel mRNAs characterised here can increase the probative value of mRNA typing results by expanding the panel of useful forensic body fluid markers. Larger and improved multiplex systems could be developed, incorporating some or all of the above markers in addition to well-known transcripts.

### Example 2: Multiplex Testing

### Materials and Methods

### Sample Collection

Human bodily samples were obtained from healthy volunteers with full informed consent. Samples for specificity testing included circulatory blood, liquid saliva, semen (containing spermatozoa), azoospermic seminal fluid, menstrual fluid, and vaginal material for RNA, as well as blood from a male individual for DNA. Donors were between 24 and 53 years of age and included males and females for circulatory blood and saliva. Blood was placed on sterile Cultiplast® rayon swabs (LP Italiana SPA, Milano, Italy) in aliquots between 5-0.05 μL. Saliva and semen were deposited on swabs in aliquots of 10-0.25 μL, and 2-0.25 μL, respectively. Semen donors included two azoospermic individuals. MF and VM were obtained by volunteers themselves using swabs provided for them. Volunteers donating semen, menstrual fluid, or vaginal material were asked to abstain from sexual intercourse for one week prior to sample collection.

Mixtures of body fluids were prepared by adding increasing volumes of blood or semen (1 μL, 5 μL, and 10 μL) to 1/3 of a MF swab. Likewise, 1 μL, 5 μL, or 10 μL saliva was added to 1/3 of a VM swab, as well as to 2 μL semen placed on a swab. Finally, 2 μL semen and 10 μL saliva were added to a VM swab. All samples were prepared in duplicate, except for mixtures of MF and semen.

For the sensitivity study, decreasing volumes of circulatory blood (2.5-0.05 μL), saliva (5-0.25 μL), semen (1-0.05 μL), and seminal fluid (1-0.05 μL) were extracted, whereas decreasing RNA concentrations were reverse transcribed for MF and VM. All samples were prepared in duplicate and reverse transcribed using 10 μL and 1 μL RNA.

For the species specificity testing, circulatory blood and saliva were collected opportunistically from 24 species, including primates, monkeys, birds, cat, chicken, dog, guinea pig, otter, rabbit, sheep, and wallaby. Samples were kindly supplied by pet owners, veterinarians, and Auckland Zoo staff. A total of 41 samples (20 circulatory blood and 21 saliva/buccal mucosa) were obtained. DNA fractions collected during extraction were retained from all species.

### DNA/RNA Co-Extraction and RNA Purification

DNA/RNA co-extractions were carried out as described previously [53] using the Promega® DNA IQ™ System (Promega Corporation, Madison, Wis., USA), following the manufacturer's instructions. DNA was eluted in 50 μL elution buffer.

Crude RNA lysates were further processed using the ReliaPrep™ RNA Cell Miniprep System (Promega) as published [53]. RNA was eluted in 45 μL nuclease-free water. Purified RNA samples were immediately DNase treated using the TURBO DNAfree™ Kit (Ambion®). The manufacturer's instructions were followed, adding 4.5 μL 10× TURBO DNase Buffer and 2 μL TURBO™ DNase to each sample.

### Quantification of RNA and DNA Samples

RNA samples of human origin were quantified using the Quantifiler® Human DNA Quantification Kit (Applied Biosystems®) as described in [53]. If residual genomic DNA was detected in an RNA sample, the extract was again DNase treated and re-quantified. This was repeated (no more than three times) until no human genomic DNA was detectable in both quantification duplicates of the same sample.

The DNA concentration of the human body fluid sample was determined via use of the Quantifiler® System as described above. Animal DNA was quantified using the Qubit® 2.0 Fluorometer and Qubit® dsDNA High Sensitivity Assay Kit (Molecular Probes® by Life Technologies, Inc.). Reactions were performed according to the manufacturer's instructions using 2 μL of each sample.

### Reverse Transcription of RNA Samples

DNA-free RNA samples (10 μL or 1 μL) were reverse transcribed using the High Capacity cDNA Reverse Transcription Kit (Applied Biosystems®) according to the manufacturer's instructions. Each reaction comprised a total volume of 20 μL.

### Primer and Multiplex Design

Primers for HBD, SLC4A1, FDCSP, HTN3, MMP10, STC1, and CYP2B7P were designed to target transcript stable regions (StaRs) [23] using the OligoAnalyzer 3.1 online tool (Integrated DNA Technologies, Inc., Coralville, Iowa, USA). Sequencing coverage maps were viewed in Geneious v.5.6.7 (Biomatters Ltd., Auckland, New Zealand) and regions of high read coverage were selected for primer design. Primers for TNP1, KLK2, and MSMB were designed using conventional primer design strategy, whereas primers for PRM1 were adopted from the literature [94]. The specificity of all primers to their intended mRNA target was verified using Primer-BLAST (National Center for Biotechnology Information, U.S. National Library of Medicine, Bethesda, Md., USA).

Primers were compiled into three multiplex assays:
1) a duplex combining FDCSP and HTN3 (multiplex D),
2) a quadruplex including HBD, SLC4A1, MMP10, and STC1 (multiplex Q), and
3) a pentaplex combining PRM1, TNP1, KLK2, MSMB, and CYP2B7P (multiplex P).

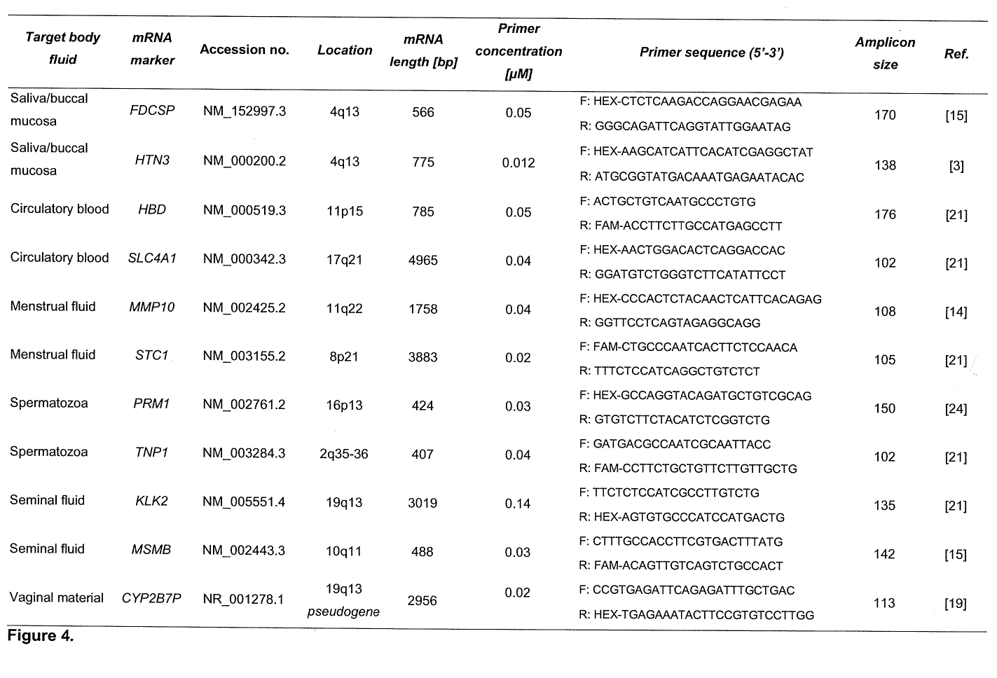

Optimized primer concentrations were as follows:
1) 0.05 μM FDCSP and 0.012 μM HTN3,
2) 0.05 μM HBD, 0.04 μM SLC4A1, 0.04 μM MMP10, and 0.02 μM STC1, and
3) 0.03 μM PRM1, 0.04 μM TNP1, 0.14 μM KLK2, 0.03 μM MSMB, and 0.02 μM CYP2B7P.
   Primer sequences and expected amplicon sizes are listed in FIG. 4.

### Multiplex Endpoint PCR

PCR was performed on a GeneAmp PCR System 9700 in 25 μL reactions using 12.5 μL Qiagen® Multiplex PCR buffer, 2.5 μL primer mix, and 2 μL or 10 μL cDNA. Where 2 μL cDNA was used, the total reaction volume of 25 μL was achieved by the addition of 8 μL nuclease-free water. DNA samples were amplified using an input of approximately 1.5 ng, performing dilutions where necessary. DNA from blood was preferred over saliva due to the potential of co-extracting plant material in animal saliva samples.

Amplification negative controls (ANEG) comprised nuclease-free water in place of cDNA. Amplification positive controls (APOS) were prepared from pooled cDNA from four known samples per body fluid (buccal samples for multiplex D, menstrual fluid samples for multiplex Q, and semen and vaginal material samples for multiplex P) from various individuals. Each sample was tested for the presence of all target mRNAs prior to pooling. The resulting APOS samples were diluted in TE buffer to display peak heights of around 10,000 relative fluorescent units (RFU) without over-amplification.

The protocol for RT-PCR [1] was optimized by adjusting the annealing temperature and duration, as well as the final elongation time. To allow for the use of a universal amplification protocol, PCR conditions were selected as those which maximised target signals simultaneously in all three multiplex assays. Final optimized PCR conditions were:
initial denaturation at 95° C. for 15 min, followed by
35 cycles of 94° C. for 30 s, 60° C. for 3 min and 72° C. for 1 min,
final elongation at 72° C. for 10 min, and
cooling down to 4° C.

### Capillary Electrophoresis and Data Analysis

PCR products were separated on a 3500xL Genetic Analyzer (Applied Biosystems®). Briefly, 9.6 μL Hi-Di™ was mixed with 0.4 μL GeneScan™ 600 LIZ® dye Size Standard v2.0 (Applied Biosystems®) per sample, to which 2 μL of PCR product was added. One amplification positive control and one negative control were injected per every 22 samples analysed. Samples were injected at a voltage of 1.2 kV for 24 s. Results were analysed using GeneMapper® ID-X v.1.5 (Applied Biosystems®) and an analytical threshold of 50 RFU.

### Results

### Species Specificity

As shown in Table 3, all primate blood samples (except squirrel monkey) produced signals for the two circulatory blood markers. Most signals were observed for HBD, particularly in primate and rabbit blood. This was expected, since primate mRNA is very similar to human mRNA (e.g., 98% sequence identity between human and northern white-cheeked gibbon HBD [54]). Furthermore, haemoglobins are widely expressed in many bird and mammal species, although some only possess a pseudogene [55]. STC1 was only observed in the grey-headed flying fox sample. A signal the size of MMP10 plus 2 bp was detected in cat blood. Amplification products of the same size as CYP2B7P were detected in the siamang gibbon and cotton-top tamarin samples. This could be the result of CYP2B7P expression in primates, whereas humans only possess a pseudogene. The cotton-top tamarin sample also displayed an off-scale MSMB peak.

The majority of animal saliva samples did not indicate the presence of target amplification products. Only the bonnet macaque sample produced FDCSP, SLC4A1, MSMB, and CYP2B7P signals. FDCSP was also detected in the squirrel monkey and dog samples. The cotton-top tamarin sample displayed MSMB and CYP2B7P peaks, which were also observed in blood. These were unlikely to originate from residual DNA, since the amplification of DNA did not give rise to comparable signals. Therefore, MSMB or low levels of CYP2B7P mRNA may be present in circulatory blood or saliva of some primate species.

TABLE 3
Specificity of the three multiplex assays for circulatory blood and saliva
collected from 24 species.

| | FDCSP | HTN3 | HBD | SLC4A1 | MMP10 | STC1 | PRM1 | TNP1 | KLK2 | MSMB | CYP2B7P |
|---|---|---|---|---|---|---|---|---|---|---|---|
| Species | | | | | | | | | | | |
| (blood samples) | | | | | | | | | | | |
| Bonnet macaque | .. | .. | 3204 | 92145 | .. | .. | .. | .. | .. | .. | .. |
| Cotton-top tamarin | .. | .. | 11979 | 19404 | .. | .. | .. | .. | .. | 96135 | 2382 |
| Pygmy marmoset | .. | .. | 97323 | 9726 | .. | .. | .. | .. | .. | .. | .. |
| Siamang gibbon | .. | .. | 97296 | 92955 | .. | .. | .. | .. | .. | .. | 1791 |
| Spider monkey | .. | .. | 11436 | 924¹ | .. | .. | .. | .. | .. | .. | .. |
| Squirrel monkey | .. | .. | 29073 | .. | .. | .. | .. | .. | .. | .. | .. |
| Capybara | .. | .. | .. | .. | .. | .. | .. | .. | .. | .. | .. |
| Cat | .. | .. | 1134 | .. | 723² | .. | .. | .. | .. | .. | .. |
| Dog | .. | .. | .. | .. | .. | .. | .. | .. | .. | .. | .. |
| Grey-headed flying fox | .. | .. | 135 | .. | .. | 10395 | .. | .. | .. | .. | .. |
| Lovebird | .. | .. | .. | .. | .. | .. | .. | .. | .. | .. | .. |
| Meerkat | 144¹ | .. | .. | .. | .. | .. | .. | .. | .. | .. | .. |
| Otter | .. | .. | 5217 | .. | .. | .. | .. | .. | .. | .. | .. |
| Porcupine | .. | .. | .. | .. | .. | .. | .. | .. | .. | .. | .. |
| Rabbit | .. | .. | 96063 | .. | .. | .. | .. | .. | .. | .. | .. |
| Red panda | .. | .. | 924 | .. | .. | .. | .. | .. | .. | .. | .. |
| Tasmanian devil | .. | .. | .. | .. | .. | .. | .. | .. | .. | .. | .. |
| Tiger | .. | .. | .. | .. | .. | .. | .. | .. | .. | .. | .. |
| Wallaby | .. | .. | 171 | .. | .. | .. | .. | .. | .. | .. | .. |
| Wood duck | .. | 6972 | 255 | 822¹ | .. | .. | .. | .. | .. | .. | .. |
| ENEG³ | .. | .. | .. | .. | .. | .. | .. | .. | .. | .. | .. |
| APOS | 24518 | 16888 | 4017 | 13919 | 12540 | 7815 | 8691 | 747 | 17583 | 27125 | 12753 |
| ANEG | .. | .. | .. | .. | .. | .. | .. | .. | .. | .. | .. |
| Species | | | | | | | | | | | |
| (saliva samples) | | | | | | | | | | | |
| Bonnet macaque | 91815 | .. | .. | 8814 | .. | .. | .. | .. | .. | 11795 | 1365 |
| Cotton-top tamarin | .. | .. | .. | .. | .. | .. | .. | .. | .. | 34483 | 976 |
| Golden lion tamarin | .. | .. | .. | .. | .. | .. | .. | .. | .. | .. | .. |
| Pygmy marmoset | .. | .. | .. | .. | .. | .. | .. | .. | .. | .. | .. |
| Spider monkey | .. | .. | .. | .. | .. | .. | .. | .. | .. | .. | .. |
| Squirrel monkey | 180 | .. | .. | .. | .. | .. | .. | .. | .. | .. | .. |
| Capybara | .. | .. | .. | .. | .. | .. | .. | .. | .. | .. | .. |
| Cat | .. | .. | .. | .. | .. | .. | .. | .. | .. | .. | .. |
| Chicken | .. | .. | .. | .. | .. | .. | .. | .. | .. | .. | .. |
| Dog | 8604 | .. | .. | .. | .. | .. | .. | .. | .. | .. | .. |
| Grey-headed flying fox | .. | .. | .. | .. | .. | .. | .. | .. | .. | .. | .. |
| Guinea pig | .. | .. | .. | .. | .. | .. | .. | .. | .. | .. | .. |
| Lovebird | .. | .. | .. | .. | .. | .. | .. | .. | .. | .. | .. |
| Otter | .. | .. | .. | .. | .. | .. | .. | .. | .. | .. | .. |
| Rabbit⁴ | .. | .. | .. | .. | .. | .. | .. | .. | .. | .. | .. |
| Red panda | .. | .. | .. | .. | .. | .. | .. | .. | .. | .. | .. |
| Sheep | .. | .. | .. | .. | .. | .. | .. | .. | .. | .. | .. |
| Tasmanian devil | .. | .. | .. | .. | .. | .. | .. | .. | .. | .. | .. |
| Tiger | .. | .. | .. | .. | .. | .. | .. | .. | .. | .. | .. |
| Wallaby | .. | .. | .. | .. | .. | .. | .. | .. | .. | .. | .. |
| Wood duck | .. | .. | .. | .. | .. | .. | .. | .. | .. | .. | .. |
| ENEG³ | .. | .. | .. | .. | .. | .. | .. | .. | .. | .. | .. |
| APOS | 24518 | 16888 | 8926 | 7023 | 10442 | 3283 | 3676 | 2131 | 12182 | 12411 | 7392 |
| ANEG | .. | .. | .. | .. | .. | .. | .. | .. | .. | .. | .. |

¹Observed product sized 1-2 bp smaller than expected
²Observed product sized 1-2 bp larger than expected.
³Extraction negative control.
⁴Absence of signal was expected, since the DNA concentration from the same sample was below the detection threshold.

The remaining signals may have originated from amplification of trace amounts of mRNA due to overloading PCR reactions, since sample volumes were difficult to estimate. Additional amplification products outside expected marker positions were observed in most samples. These possibly resulted from unspecific primer binding and may be avoided by further increasing the annealing temperature [56].

Animal DNA samples mostly displayed raised baselines and numerous unspecific amplification products of peak heights below 1,000 RFU. Although some peaks were of the same size as expected marker products, this likely occurred by coincidence. The appearance of several unexpected signals in combination with a noisy baseline was a good indicator for the presence of DNA. Signals exceeding 4,000 RFU were observed for TNP1 from bonnet macaque, pygmy marmoset, siamang gibbon, and spider monkey. This may be due to the fact that the TNP1 primers amplified DNA. In addition, MSMB was observed in the golden lion tamarin sample.

### Body Fluid Specificity

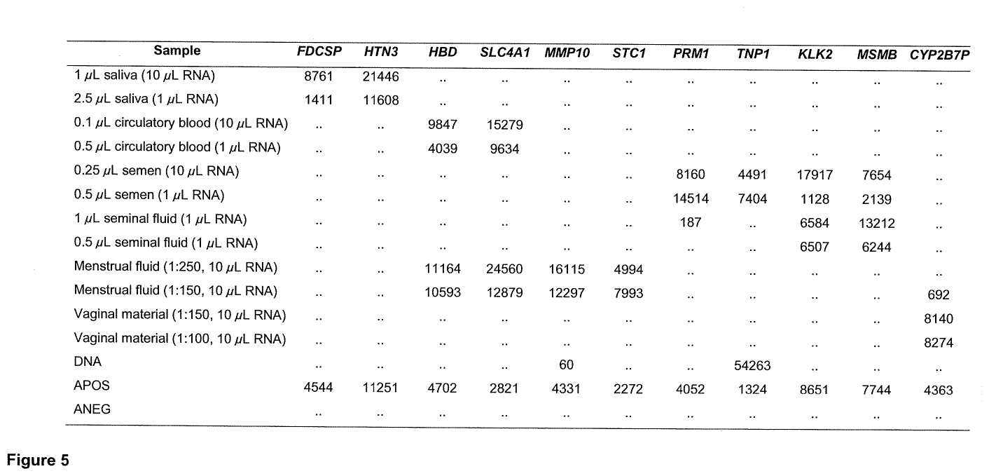

FIG. 5 shows that no cross-reactions from non-target body fluids were observed, except for a PRM1 signal (187 RFU) in an azoospermic semen sample. However, spermatozoa can sometimes be present in semen following vasectomy [57]. In addition, CYP2B7P was undetected in one menstrual fluid sample. Cervical mucus and vaginal discharge contribute little to the total fluid volume lost during menstruation [58], hence corresponding markers may be present below the detection limit.

The human DNA sample produced a peak of 60 RFU for MMP10 (FIG. 5). This signal could be attributed to elevated baseline and can be avoided by raising the analytical threshold. In addition, TNP1 was amplified (54,263 RFU). This was likely due to the fact that the TNP1 forward primer was placed across an exon/exon boundary, with only seven bases aligning to a different exon than the reverse primer. TNP1 therefore cannot distinguish between mRNA and DNA templates, and a TNP1 signal is not confirmatory for the presence of semen. Reverse transcriptase negative (RT−) controls can help to verify whether residual genomic DNA may have contributed to a signal. Furthermore, massively parallel sequencing (MPS) could determine amplicon sequences and thus distinguish between templates in the future.

To evaluate the potential for false positives due to excessive sample input, ten samples per body fluid from five donors (10 μL saliva, 5 μL blood, 2 μL semen, and whole MF and VM swabs) were amplified. Target marker signals were typically over-amplified, i.e. in the 70,000-90,000 RFU range (Table 4). Exceptions were HTN3 in saliva from donor A, menstrual fluid samples from donor R, and CYP2B7P in menstrual fluid samples, which were considerably lower. This corroborates previous findings of high variation in transcript abundance among individuals and samples [4,10].

Low-level cross-reactions were observed for all markers and body fluids, except for MMP10, STC1, PRM1, and MSMB in circulatory blood, HBD, SLC4A1, PRM1, and KLK2 in saliva, and HTN3 in menstrual fluid. This confirms previous reports of low transcript abundance in non-target body fluids for all currently known mRNAs [3,39,10,14]. Most signals were below 500 RFU and would likely be absent if a suitable analytical threshold were applied and target marker peaks were in the ideal range of 4,000-12,000 RFU on a 3500xL instrument. However, cross-reactions exceeding 10,000 RFU were observed for FDCSP in two MF samples from two donors, for MMP10 in two saliva, one semen, and three VM samples, as well as for MSMB in one VM sample. This demonstrates relatively higher FDCSP, MMP10, and MSMB transcript abundance in non-target body fluids and consequently lower specificity compared to the remaining mRNAs. Nevertheless, no cross-reactions were observed at ideal sample input (FIG. 5).

TABLE 4
Body fluid specificity of the three multiplex assays using excessive RNA and cDNA input.

| | FDCSP | HTN3 | HBD | SLC4A1 | MMP10 | STC1 | PRM1 | TNP1 | KLK2 | MSMB | CYP2B7P |
|---|---|---|---|---|---|---|---|---|---|---|---|
| Saliva | | | | | | | | | | | |
| Donor N - sample 1 | 93714 | 97272 | .. | .. | .. | 282 | .. | 144² | .. | .. | .. |
| Donor N - sample 2 | 92152 | 95698 | .. | .. | .. | 267 | .. | .. | .. | 2889 | .. |
| Donor T - sample 1 | 89502 | 95826 | .. | .. | 6687 | 162 | .. | 189 | .. | 1512 | .. |
| Donor T - sample 2 | 90609 | 97206 | .. | .. | 7206 | 105 | .. | .. | .. | 6792 | 411 |
| Donor M - sample 1 | 93675 | 97530 | .. | .. | 22950 | 129 | .. | 162¹ | .. | 1896 | .. |
| Donor M - sample 2 | 90129 | 93996 | .. | .. | 6168 | 159 | .. | 198¹ | .. | 1356 | 516 |
| Donor P - sample 1 | 90780 | 95970 | .. | .. | 16875 | .. | .. | .. | .. | .. | .. |
| Donor P - sample 2 | 88005 | 95583 | .. | .. | 7191 | .. | .. | .. | .. | .. | .. |
| Donor A - sample 1 | 90423 | 70950 | .. | .. | .. | 309 | .. | 141 | .. | .. | .. |
| Donor A - sample 2 | 89871 | 72678 | .. | .. | 3078 | 213 | .. | 147² | .. | .. | .. |
| APOS | 7621 | 25905 | 1523 | 5725 | 5170 | 2258 | 3850 | 1574 | 15293 | 9162 | 4459 |
| ANEG | .. | .. | .. | .. | .. | .. | .. | .. | .. | .. | .. |
| Circulatory blood | | | | | | | | | | | |
| Donor N - sample 1 | .. | .. | 97215 | 89445 | .. | .. | .. | 798 | .. | .. | 474 |
| Donor N - sample 2 | 73 | 61 | 97023 | 89022 | .. | .. | .. | .. | .. | .. | 651 |
| Donor T - sample 1 | .. | .. | 97443 | 90954 | .. | .. | .. | 96² | .. | .. | 678 |
| Donor T - sample 2 | .. | .. | 97548 | 92568 | .. | .. | .. | 162¹ | .. | .. | .. |
| Donor M - sample 1 | .. | .. | 97356 | 94188 | .. | .. | .. | 201¹ | .. | .. | .. |
| Donor M - sample 2 | .. | .. | 97560 | 91539 | .. | .. | .. | 273² | .. | .. | .. |
| Donor P - sample 1 | 54 | .. | 97590 | 91941 | .. | .. | .. | 207¹ | .. | .. | 561 |
| Donor P - sample 2 | 123 | 60 | 95763 | 90180 | .. | .. | .. | 162¹ | 51 | .. | .. |
| Donor A - sample 1 | 132 | .. | 97464 | 90681 | .. | .. | .. | 120² | .. | .. | .. |
| Donor A - sample 2 | .. | .. | 97746 | 91569 | .. | .. | .. | .. | .. | .. | .. |
| APOS | 7621 | 25905 | 3245 | 8669 | 6780 | 1451 | 3850 | 1574 | 15293 | 9162 | 4459 |
| ANEG | .. | .. | .. | .. | .. | .. | .. | .. | .. | .. | .. |
| Semen | | | | | | | | | | | |
| Donor F - sample 1 | 147 | 87 | .. | .. | 10245 | 108 | 97239 | 96120 | 94941 | 97650 | .. |
| Donor F - sample 2 | 144 | 69 | .. | .. | 486 | 1905 | 95214 | 95703 | 92271 | 97542 | .. |
| Donor O - sample 1 | .. | .. | 2181 | .. | 4191 | .. | 93078 | 95721 | 90954 | 97437 | 1341 |
| Donor O - sample 2 | .. | .. | .. | .. | .. | 2175 | 94923 | 95535 | 90402 | 97380 | .. |
| Donor T - sample 1 | .. | .. | .. | .. | .. | 132¹ | 92289 | 96165 | 90306 | 97608 | .. |
| Donor T - sample 2 | .. | .. | .. | .. | .. | .. | 97542 | 96648 | 95403 | 97752 | .. |
| Donor S - sample 1 | .. | .. | .. | 231¹ | .. | 132¹ | .. | .. | 93138 | 97542 | .. |
| Donor S - sample 2 | .. | .. | .. | .. | .. | 135¹ | .. | .. | 90924 | 97254 | .. |
| Donor U - sample 1 | .. | .. | .. | .. | .. | 132 | .. | .. | 89532 | 97431 | 315 |
| Donor U - sample 2 | 138 | 51 | .. | .. | 69 | 2217 | .. | .. | 89925 | 97062 | 1101 |
| APOS | 7621 | 25905 | 1523 | 5725 | 5170 | 2258 | 9116 | 2547 | 26109 | 18068 | 12395 |
| ANEG | .. | .. | .. | .. | .. | .. | .. | .. | .. | .. | .. |
| Menstrual fluid | | | | | | | | | | | |
| Donor A - sample 1 | 2942 | .. | 74133 | 70018 | 71260 | 75906 | .. | .. | .. | .. | 2856 |
| Donor A - sample 2 | .. | .. | 73777 | 68184 | 69349 | 75952 | 91 | 246 | 200 | 188 | 7209 |
| Donor M - sample 1 | 3169 | .. | 80809 | 73771 | 74882 | 82648 | .. | .. | 150 | .. | 5929 |
| Donor M - sample 2 | 13634 | .. | 81136 | 75101 | 76717 | 83062 | .. | 4502 | .. | .. | 18981 |
| Donor C - sample 1 | 13709 | .. | 73629 | 67180 | 68632 | 75493 | .. | 4172 | .. | .. | 30405 |
| Donor C - sample 2 | 8568 | .. | 76050 | 70476 | 71121 | 77740 | .. | .. | 130 | .. | 27420 |
| Donor P - sample 1 | 1986 | .. | 82946 | 79066 | 79609 | 84603 | .. | .. | 156 | .. | 72072 |
| Donor P - sample 2 | .. | .. | 95502 | 92733 | 93350 | 97088 | .. | .. | 118 | .. | 21720 |
| Donor R - sample 1 | 75 | .. | 59778 | 56261 | 61697 | 38894 | 101 | 311 | 246 | 201 | 18882 |
| Donor R - sample 2 | 61 | .. | 47644 | 34200 | 75738 | 28891 | .. | .. | .. | 2992 | 20818 |
| APOS | 7621 | 25905 | 3245 | 8669 | 6780 | 1451 | 9116 | 2547 | 26109 | 18068 | 12395 |
| ANEG | .. | .. | .. | .. | .. | .. | .. | .. | .. | .. | .. |
| Vaginal material | | | | | | | | | | | |
| Donor A - sample 1 | .. | .. | .. | .. | 4103 | .. | .. | .. | .. | .. | 73572 |
| Donor A - sample 2 | .. | .. | 112 | 235 | 66 | .. | .. | .. | 66 | .. | 61708 |
| Donor M - sample 1 | .. | .. | .. | .. | 30624 | 1032 | 96 | 137 | 188 | 10189 | 76121 |
| Donor M - sample 2 | .. | .. | .. | .. | 17068 | 2059 | .. | 88 | 77 | 4127 | 68506 |
| Donor P - sample 1 | .. | .. | .. | .. | 7065 | .. | .. | .. | 80 | .. | 73504 |
| Donor P - sample 2 | .. | .. | .. | .. | 5800 | 436 | .. | .. | 107 | .. | 74947 |
| Donor Q - sample 1 | .. | .. | .. | .. | 1661 | .. | .. | .. | 1967 | 2699 | 90156 |
| Donor Q - sample 2 | 52 | .. | .. | .. | 56 | .. | 84 | 159 | 129 | 1815 | 87435 |
| Donor R - sample 1 | 76 | .. | .. | .. | 20848 | 267 | .. | .. | 310 | .. | 80585 |
| Donor R - sample 2 | 3455 | 74 | 110 | .. | 7284 | 1079 | .. | .. | .. | 7942 | 84383 |
| ENEG | .. | .. | .. | .. | .. | .. | .. | .. | .. | .. | .. |
| APOS | 7621 | 25905 | 3245 | 8669 | 6780 | 1451 | 9116 | 2547 | 26109 | 18068 | 12395 |
| ANEG | .. | .. | .. | .. | .. | .. | .. | .. | .. | .. | .. |

¹Observed product sized 1-2 bp smaller than expected.
²Observed product sized 1-2 bp larger than expected.

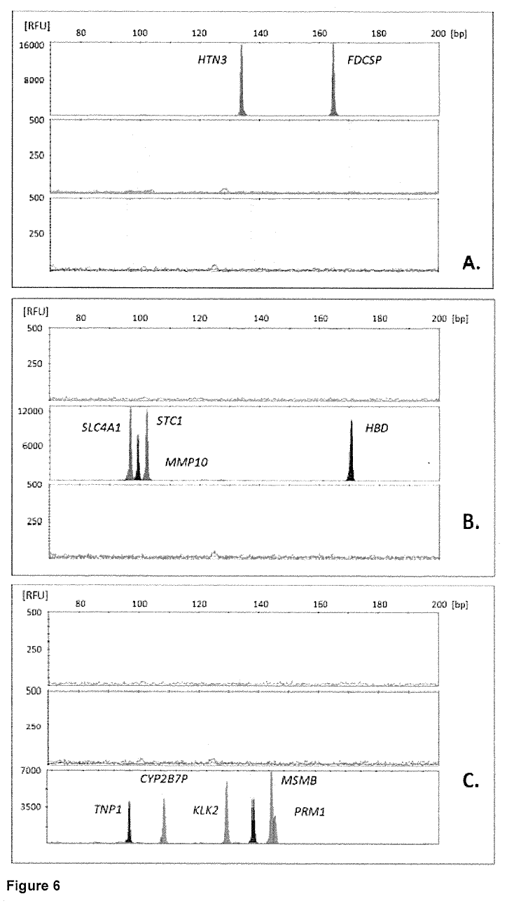

It is therefore essential to limit sample input amounts and avoid over-amplification, although this may result in overlooking minor components of body fluid mixtures. HTN3, HBD, SLC4A1, and PRM1 appeared to be the most specific markers. Examples of electropherograms for the three multiplex assays are shown in FIG. 6.

### Sensitivity

The lower limit of detection (LOD) for the three multiplexes was approximately 0.5 μL saliva (multiplex D), 0.05 μL circulatory blood (multiplex Q), 0.05 μL semen containing spermatozoa (multiplex P), and 0.25 μL azoospermic seminal fluid (multiplex P) using 10 μL RNA for cDNA synthesis. For MF (multiplex Q) and VM (multiplex P), the LOD was approximately 1/50^{th} of the RNA obtained from a whole swab, using 1 μL RNA for cDNA synthesis. These results were similar to other forensic multiplex systems [3,1,39,5,59].

### Precision

The precision of the three multiplexes was evaluated by triplicate amplification of the same cDNA samples. Standard deviations (σ) and coefficients of variation (CV), expressed as σ divided by the mean, were calculated from resulting peak heights.

The saliva markers displayed dispersion around the mean of 67% and 39% for FDCSP, and 77% and 103% for HTN3. This demonstrates a higher level of variability around the mean for HTN3, and moderate to low precision for both markers. Variability ranged between 8% and 49% for HBD, and between 18% and 36% for SLC4A1. Both markers therefore showed higher precision than the saliva markers. Less dispersion appeared to occur in MF samples. MMP10, STC1, and CYP2B7P showed variability between 21-24%, 14-16%, and 18-19%, respectively. These values demonstrate moderate to good levels of precision among replicates and samples, particularly for STC1. Variability ranged between 14-93% for PRM1, 7-53% for TNP1, 14-141% for KLK2, and 16-51% for MSMB. The high dispersion of KLK2 in one semen sample (141%) was due to failure of amplification in two replicates. KLK2 was also undetected in one replicate of a second semen sample, whereas all other mRNAs were consistently detected. Although high variability of peak heights is expected for mRNA analysis [60], further research including a greater number of replicates may determine CV values more precisely.

### The Effect of Multiplexing

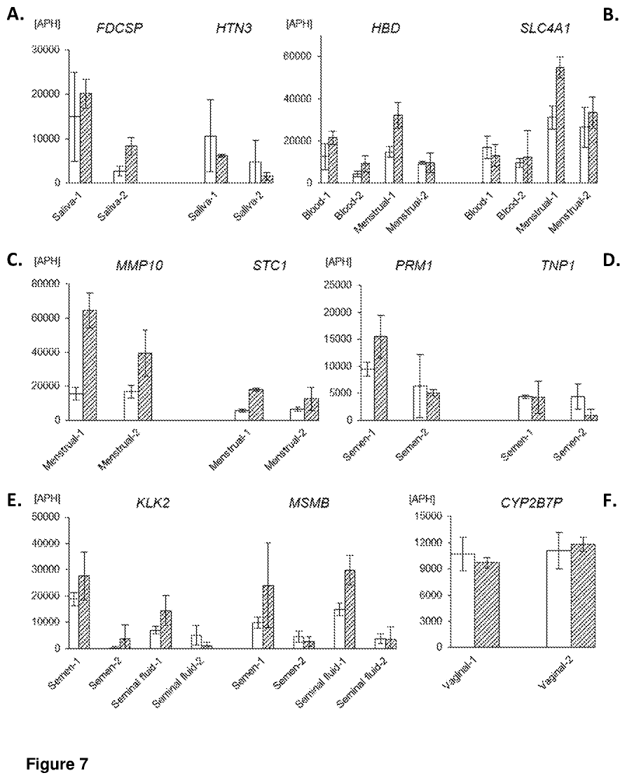

To investigate the effect that multiplexing has on target detection, 12 samples, i.e. two per body fluid, were amplified for a total of three replicates in both multiplex and uniplex reactions. All samples had previously shown ideal peak heights in multiplex amplifications. As FIG. 7 shows, only HTN3 exclusively produced higher signals in multiplex compared to uniplex. For most markers and samples, higher average peak heights (APH) were obtained in uniplex reactions. This was expected due to the reduced competition among primer sets in uniplex amplifications [56]. The strongest negative effect was observed for MMP10 and SLC4A1. APH were up to 4.1- and 1.8-fold lower in multiplex compared to uniplex reactions, respectively. This was likely the result of low heterodimerisation values between primers (ΔG≥−9.76 kcal/mole). Interestingly however, differences in APH for SLC4A1 and HBD were more pronounced in MF than in circulatory blood.

Whereas no clear tendency towards increased signals in uni- or multiplex was observed for PRM1, TNP1 appeared to perform slightly better in multiplex. This mRNA was consistently detected in multiplex, while two uniplex replicates failed to amplify. KLK2 and MSMB respectively were also undetected in four and two of 12 replicates using uniplex reactions, whereas only three and zero replicates failed in multiplex. The effect of multiplexing for CYP2B7P was negligible, although standard deviations were slightly higher in multiplex.

In 60% of 30 marker observations averaged from triplicate amplifications, the target markers exhibited less peak height variance in multiplex than in uniplex (data not shown). TNP1, KLK2, and MSMB exclusively showed higher precision in multiplex. Thus, while multiplexing exerted a negative effect on absolute peak height and therefore target detection, the markers had a tendency towards increased precision and consistent amplification in multiplex. The loss in peak height due to multiplexing was counteracted by the adjustment of primer concentrations, which balanced signals among markers within the same multiplex.

### Resolution of Body Fluid Mixtures

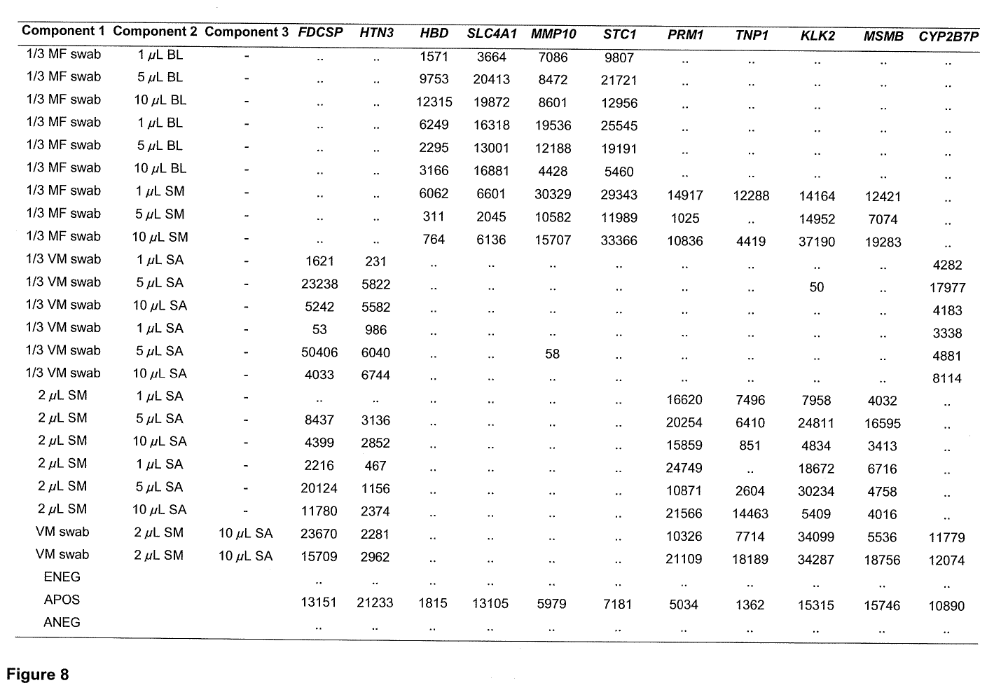

All body fluid mixtures were correctly identified, except for one sample of 1 μL saliva mixed with 2 μL semen (FIG. 8). Using the undiluted cDNA sample derived from a 1:8 dilution of the extracted RNA, FDCSP and HTN3 reached 5,829 RFU and 3,135 RFU, whereas the semen markers ranged between 11,521 RFU for MSMB and 40,745 RFU for KLK2. The circulatory blood and MF markers were undetected in both amplifications. The additional dilution of the cDNA sample to adjust peak heights of the semen markers to the ideal 4,000-12,000 RFU range resulted in loss of signal for the saliva markers. This implies that uneven mixtures with an abundant major component and a small minor component may fail to be correctly resolved.

CYP2B7P was not observed in any mixture containing menstrual fluid. This was likely because this mRNA was present below the detection threshold. TNP1 was also undetected in two samples containing semen, likely due to amplification failure. Two unexpected signals (MMP10, 58 RFU and KLK2, 50 RFU) resulted from elevated baseline. Importantly, greater body fluid volumes did not necessarily produce higher peaks. Although HBD signals increased with larger blood volumes in the first set of mixtures with MF, the second set of mixtures did not show this correlation. This probably resulted from differences in template abundance among samples.

Detection of Seminal mRNAs in Post-Coital Vaginal Samples

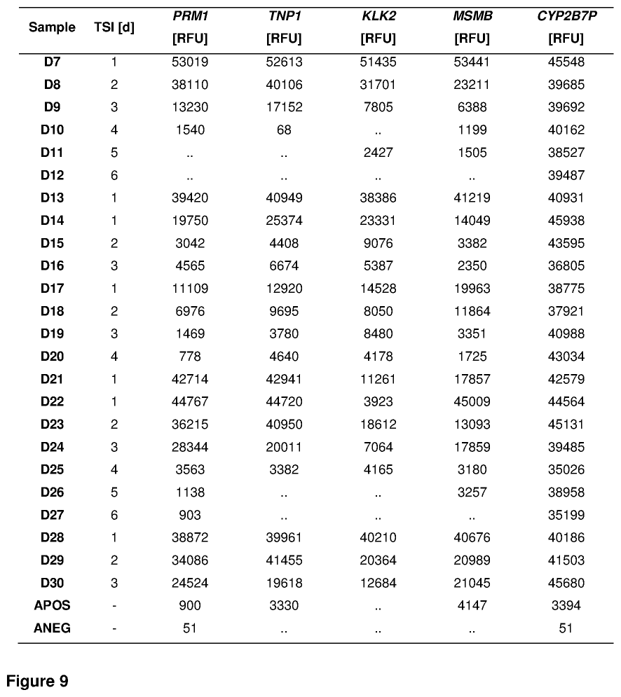

To evaluate the time frame during which seminal mRNAs could be detected on vaginal swabs collected post intercourse, 24 samples with a time since intercourse (TSI; known from self-declared information through a daily questionnaire. The donor supplied vaginal swabs on 24 consecutive days in a controlled experiment) between one and six days were amplified using multiplex P. The results are shown in FIG. 9.

All four seminal markers were consistently detected for up to three days post intercourse. The lowest signal from a TSI 3 d sample was 1,469 RFU for PRM1 (sample D19). Swabs collected four days post coitus also exhibited all four seminal markers, except sample D10, which did not show a KLK2 signal, possibly resulting from amplification failure. The two samples collected after five days (D11 and D26) each displayed MSMB and one additional marker. Whereas one sample with a TSI of six days (D12) was undetected, the second sample (D27) showed a PRM1 peak (903 RFU). Hence, the identification of seminal mRNAs in post-coital samples using the pentaplex is possible for up to six days. These results demonstrate a considerable enhancement of marker detection in post-coital samples compared to previous studies [10], which reported that the detection of seminal mRNAs was limited to samples with a TSI≤1 d.

### Stability Studies

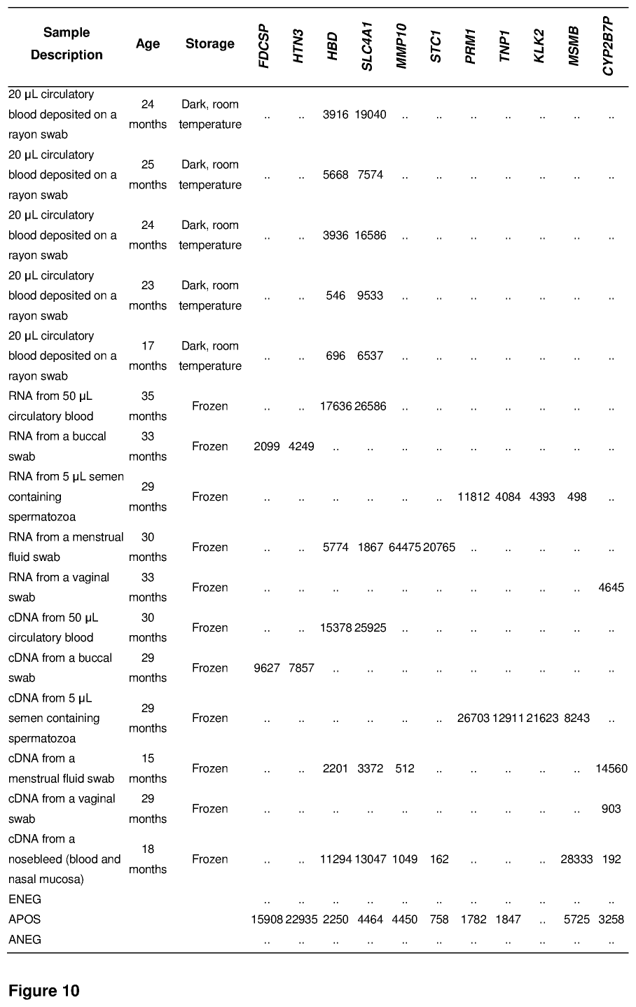

The forensic literature reported successful mRNA amplification from body fluids up to 56 years after deposition [61]. In this research, the ability to detect and identify aged body fluids, aged RNA, and aged cDNA samples was investigated. Five single-source samples for each of these three categories were selected with regard to storage time and subjected to amplification using all three multiplex assays, performing cDNA dilutions where necessary. In addition, an aged cDNA sample obtained from a nosebleed was analysed. The results are shown in FIG. 10.

All aged circulatory blood samples (17-25 months old) were correctly identified, with no cross-reactions observed. Aged RNA samples (29-35 months old) correctly exhibited all target markers, except for CYP2B7P, which was absent from the menstrual fluid sample. Aged cDNA samples (15-30 months old) were also successfully amplified, with no cross-reactions present. In the aged MF cDNA sample, the menstrual fluid marker STC1 was undetected, however a strong CYP2B7P signal provided additional confidence in the vaginal origin of the sample.

The nosebleed sample correctly exhibited signals for HBD and SLC4A1, whereas FDCSP, HTN3, PRM1, TNP1, and KLK2 were undetected. However, MMP10, STC1, CYP2B7P, and in particular MSMBwere observed. This may be problematic, since these results falsely indicate the presence of a mixture of MF and semen. One previous study also reported the amplification of CYP2B7P from nasal mucosa [39]. An analytical threshold (AT) of ≥200 RFU would prevent false positive identification of STC1 and CYP2B7P, but still allow for MMP10 and MSMB to be identified. Caution is therefore warranted in the interpretation of mRNA profiling results in the possible presence of nasal mucosa. Consequently, a MMP10 signal without detecting STC1 or CYP2B7P was considered not confirmatory for MF (unless the MMP10 peak height exceeds those of the circulatory blood markers), whereas MSMB must be accompanied by a second semen marker to confirm the presence of semen.

### Case-Type Samples

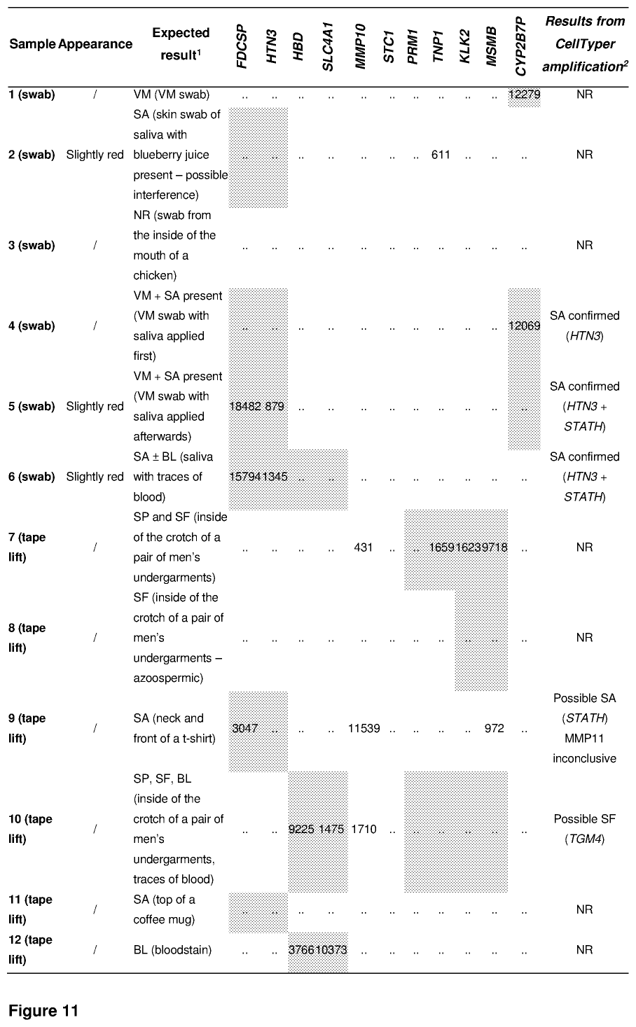

Case-type samples were processed in a blind study, in which sample sources were withheld from the researcher. A total of twelve samples (six swabs (samples 1-6) and six tape lifts (samples 7-12)) were analysed. All samples were initially amplified using 10 μL RNA and 10 μL cDNA. Subsequent cDNA dilutions were performed where necessary. Based on the results obtained in the previous sections, dilutions were required if peak heights exceeded 20,000 RFU. An analytical threshold of 400 RFU was applied for peak allocation. To compare results to a previously used method, all samples or highest dilutions thereof were also amplified using CellTyper [1]. The results are displayed in FIG. 11. RT− controls were prepared for all samples. None of these displayed any marker peaks (data not shown).

Three samples (3, 8, and 11) exhibited no marker peaks using either multiplex system. Sample 3 was a saliva sample from a chicken, and therefore correctly lacking mRNA results. Sample 8 was obtained from the inside of the crotch of a pair of men's undergarments from an azoospermic male. Hence, the presence of seminal fluid was probable. Sample 11 was a tape lift from a coffee cup and therefore expected to contain saliva. The collected material may have been insufficient to produce a result for these two samples.

Samples 1 (vaginal swab), 2 (skin swab of saliva and blueberry juice), 7 (inside of the crotch of a pair of men's undergarments), and 12 (bloodstain) were undetermined using CellTyper. The new multiplex confirmed the presence of vaginal material for sample 1. This demonstrates that Lactobacilli can be unreliable VM markers in some individuals. The detection of CYP2B7P, however, enabled determination of the source of this sample. A TNP1 signal (611 RFU) was obtained for sample 2. This result was not informative, since the signal could have originated from residual genomic DNA, although the RT− control was devoid of target signals. For sample 7, the new multiplex confirmed the presence of seminal fluid. TNP1 added strong support for the presence of semen, but should be interpreted with some caution due to the risk of amplification from DNA. MMP10 was not informative, since no corresponding mRNAs were detected. Finally, HBD and SLC4A1 were observed in sample 12 (tape lift of a bloodstain). This correctly confirmed the presence of circulatory blood. These results demonstrate improved body fluid detection using the new multiplex compared to CellTyper in three of the four samples.

Sample 4 was identified as VM using the new multiplex. Although this was a correct result, the assay failed to detect saliva as the second component (FIG. 11). In contrast, only saliva was confirmed in sample 5. This swab also comprised a mixture of saliva and VM. Saliva had been applied after (sample 5) or before (sample 4) collecting the VM sample. This could indicate that the cell lysis during the extraction process is most likely to remove cellular material from the outermost surface of a swab. Another explanation may be that the body fluid proportions were too uneven to be resolved. CellTyper detected saliva in both samples. This demonstrates higher sensitivity for saliva compared to the new multiplex. In turn, however, CellTyper failed to identify vaginal material in either sample.

Both multiplexes correctly confirmed the presence of saliva in sample 6. This sample further contained traces of blood, which neither assay detected. The possible presence of saliva was also expected for sample 9 (tape lift from the neck and upper front of a T-shirt). The new multiplex detected FDCSP, MMP10, and MSMB. These signals were insufficient to infer the presence of a body fluid. CellTyper detected corresponding marker types (STATH and MMP11), which also did not confirm a body fluid. It appears that mRNA background levels may be present on some everyday objects, which could be addressed by further research.

The improved multiplex confirmed the presence of circulatory blood in sample 10. MMP10 was also observed, but was not informative due to the absence of additional mRNAs. This sample was collected from the inside of the crotch of a pair of men's undergarments, with traces of blood applied. CellTyper detected TGM4, which indicated the presence of seminal fluid, but failed to detect blood. Overall, the new multiplex seemed to be more sensitive for circulatory blood and seminal mRNAs, whereas CellTyper was more sensitive for saliva. Further adjustment of primer concentrations may increase the sensitivity of the new multiplex for saliva.

### Conclusions

Overall, the results demonstrate successful application of the three endpoint RT-PCR multiplex assays to the identification of low abundance and aged body fluid samples, as well as to the resolution of mixtures and case-type samples. The optimized system showed similar specificity and sensitivity to other forensic multiplex assays [3,1,59], with improved results for case-type samples compared to CellTyper [1].

The species specificity study demonstrated that some primer sequences were not human-specific. HBD was frequently amplified from non-human blood samples, particularly from primates, cat, and rabbit. Large, red stains should therefore be analysed with caution. Cotton-top tamarin, bonnet macaque, and siamang gibbon samples also readily produced false positives for CYP2B7P and MSMB. Saliva samples gave fewer false positives, although dog saliva produced a FDCSP signal. The occurrence of multiple extra peaks in an electropherogram was a strong indicator of the presence of genomic DNA. The analyst should therefore carefully review the framework of the case and consider whether samples may be giving false positive results. The absence of a DNA profile can additionally indicate the presence of a non-human body fluid. If the presence of animal body fluids is suspected, additional species testing should be carried out.

Across all human body fluids, higher volumes of body fluid, RNA, and cDNA generally produced stronger signals. There was no indication of inhibitory effects at increased template amounts, although high-template samples may show increased baseline noise and non-specific peaks that could fall into marker windows. False positives readily occurred in overloaded PCR reactions. These may be caused by low-level gene expression in non-target body fluids or artefact formation resulting from non-specific primer annealing. It was therefore essential to adjust cDNA input amounts to establish marker specificity. Replicate amplifications may be useful to identify cross-reactions. RT− controls can provide additional information on whether DNA may have contributed to a signal. An analytical threshold of 400 RFU is recommended to additionally help prevent false positive marker identification.

Throughout this study, high inter-individual and inter-sample variation was observed, although the body fluids detected were consistent among replicates. This was expected due to the multitude of factors that affect gene expression [4] and the inability, at present, to measure the human-specific RNA concentration in a sample [62]. The impact of this variation was further exacerbated by low precision among replicates. Multiplexing increased overall precision, but had a detrimental effect on absolute peak height for most markers. Additionally, stochastic effects were prominent in low-template samples. Drop-out was observed for various markers at low RNA concentrations, whereas the same markers re-appeared at even lower RNA concentrations.

Mixtures of vaginal material and semen in samples collected post intercourse were successfully identified for up to six days. It is important to note that mixtures with uneven proportions may not be fully resolved. Whereas the major component was successfully detected in all mixtures analysed, the minor component(s) may be undetected because of low abundance, resulting in signals below the detection threshold. However, this is a general limitation of the technique. In view of the above results, the developed multiplex system provides a reliable and sensitive method for body fluid and cell type assessment of forensic samples.

### REFERENCES

[1] R. I. Fleming, S. Harbison, The development of a mRNA multiplex RT-PCR assay for the definitive identification of body fluids, Forensic Sci Int Genet. 4 (2010) 244-256.
[2] J. Juusola, J. Ballantyne, Messenger RNA profiling: A prototype method to supplant conventional methods for body fluid identification, Forensic Sci Int. 135 (2003) 85-96.
[3] A. Lindenbergh, M. de Pagter, G. Ramdayal, M. Visser, D. Zubakov, M. Kayser, T. Sijen, A multiplex (m)RNA-profiling system for the forensic identification of body fluids and contact traces, Forensic Sci Int Genet. 6 (2012) 565-577.
[4] T. Sijen, Molecular approaches for forensic cell type identification: On mRNA, miRNA, DNA methylation and microbial markers, Forensic Sci Int Genet. 18 (2015) 21-32.
[5] J. Juusola, J. Ballantyne, Multiplex mRNA profiling for the identification of body fluids, Forensic Sci Int. 152 (2005) 1-12.
[6] J. Juusola, J. Ballantyne, mRNA profiling for body fluid identification by multiplex quantitative RT-PCR, J Forensic Sci. 52 (2007) 1252-1262.
[7] C. Haas, B. Klesser, C. Maake, W. Bar, A. Kratzer, mRNA profiling for body fluid identification by reverse transcription endpoint PCR and realtime PCR, Forensic Sci Int Genet. 3 (2009) 80-88.
[8] C. Haas, E. Hanson, W. Bär, R. Banemann, A. Bento, A. Berti, E. Borges, C. Bouakaze, A. Carracedo, M. Carvalho, mRNA profiling for the identification of blood—results of a collaborative EDNAP exercise, Forensic Sci Int Genet. 5 (2011) 21-26.
[9] C. Haas, E. Hanson, A. Kratzer, W. Bar, J. Ballantyne, Selection of highly specific and sensitive mRNA biomarkers for the identification of blood, Forensic Sci Int Genet. 5 (2011) 449-458.
[10] A. D. Roeder, C. Haas, mRNA profiling using a minimum of five mRNA markers per body fluid and a novel scoring method for body fluid identification, Int J Legal Med. 127 (2013) 707-721.
[11] M. van den Berge, A. Carracedo, I. Gomes, E. A. Graham, C. Haas, B. Hjort, P. Hoff-Olsen, O. Maronas, B. Mevag, N. Morling, H. Niederstatter, W. Parson, P. M. Schneider, D. S. Court, A. Vidaki, T. Sijen, A collaborative European exercise on mRNA-based body fluid/skin typing and interpretation of DNA and RNA results, Forensic Sci Int Genet. 10 (2014) 40-48.
[12] M. L. Richard, K. A. Harper, R. L. Craig, A. J. Onorato, J. M. Robertson, J. Donfack, Evaluation of mRNA marker specificity for the identification of five human body fluids by capillary electrophoresis, Forensic Sci Int Genet. 6 (2012) 452-460.
[13] C. Haas, E. Hanson, M. J. Anjos, R. Banemann, A. Berti, E. Borges, A. Carracedo, M. Carvalho, C. Courts, G. De Cock, M. Dotsch, S. Flynn, I. Gomes, C. Hollard, B. Hjort, P. Hoff-Olsen, K. Hribikova, A. Lindenbergh, B. Ludes, O. Maronas, N. McCallum, D. Moore, N. Morling, H. Niederstatter, F. Noel, W. Parson, C. Popielarz, C. Rapone, A. D. Roeder, Y. Ruiz, E. Sauer, P. M. Schneider, T. Sijen, D. S. Court, B. Sviezena, M. Turanska, A. Vidaki, L. Zatkalikova, J. Ballantyne, RNA/DNA co-analysis from human saliva and semen stains—results of a third collaborative EDNAP exercise, Forensic Sci Int Genet. 7 (2013) 230-239.
[14] E. K. Hanson, J. Ballantyne, Highly specific mRNA biomarkers for the identification of vaginal secretions in sexual assault investigations, Sci Justice. 53 (2013) 14-22.
[15] C. Cossu, U. Germann, A. Kratzer, W. Bär, C. Haas, How specific are the vaginal secretion mRNA-markers HBD1 and MUC4? Forensic Sci Int Gen Supplement Series. 2 (2009) 536-537.
[16] C. Nussbaumer, E. Gharehbaghi-Schnell, I. Korschineck, Messenger RNA profiling: A novel method for body fluid identification by real-time PCR, Forensic Sci Int. 157 (2006) 181-186.
[17] M. Bauer, D. Patzelt, Evaluation of mRNA markers for the identification of menstrual blood, J Forensic Sci. 47 (2002) 1278-1282.
[18] S. M. Park, S. Y. Park, J. H. Kim, T. W. Kang, J. L. Park, K. M. Woo, J. S. Kim, H. C. Lee, S. Y. Kim, S. H. Lee, Genome-wide mRNA profiling and multiplex quantitative RT-PCR for forensic body fluid identification, Forensic Sci Int Genet. 7 (2013) 143-150.
[19] D. Zubakov, E. Hanekamp, M. Kokshoorn, W. van Ijcken, M. Kayser, Stable RNA markers for identification of blood and saliva stains revealed from whole genome expression analysis of time-wise degraded samples, Int J Legal Med. 122 (2008) 135-142.
[20] R. Fang, C. F. Manohar, C. Shulse, M. Brevnov, A. Wong, O. V. Petrauskene, P. Brzoska, M. R. Furtado, Real-time PCR assays for the detection of tissue and body fluid specific mRNAs, Int Congr Ser. 1288 (2006) 685-687.
[21] Z. Wang, M. Gerstein, M. Snyder, RNA-Seq: A revolutionary tool for transcriptomics, Nat Rev Genet. 10 (2009) 57-63.
[22] M. H. Lin, D. F. Jones, R. Fleming, Transcriptomic analysis of degraded forensic body fluids, Forensic Sci Int Genet. 17 (2015) 35-42.
[23] M. H. Lin, P. P. Albani, R. Fleming, Degraded RNA transcript stable regions (StaRs) as targets for enhanced forensic RNA body fluid identification, Forensic Sci Int Genet. 20 (2016) 61-70.
[24] X. Liu, X. Yu, D. J. Zack, H. Zhu, J. Qian, TiGER: A database for tissue-specific gene expression and regulation, BMC Bioinformatics. 9 (2008) 271.
[25] J. Pan, S. Hu, D. Shi, M. Cai, Y. Li, Q. Zou, Z. Ji, PaGenBase: A pattern gene database for the global and dynamic understanding of gene function, PloS one. 8 (2013) e80747.
[26] J. Ye, G. Coulouris, I. Zaretskaya, I. Cutcutache, S. Rozen, T. L. Madden, Primer-BLAST: A tool to design target-specific primers for polymerase chain reaction, BMC Bioinformatics. 13 (2012) 134.
[27] M. H. Steinberg, G. P. Rodgers, HbA2: Biology, clinical relevance and a possible target for ameliorating sickle cell disease, Br J Haematol. 170 (2015) 781-787.
[28] J. Ross, A. Pizarro, Human beta and delta globin messenger RNAs turn over at different rates, J Mol Biol. 167 (1983) 607-617.
[29] A. lolascon, S. Perrotta, G. W. Stewart, Red blood cell membrane defects, Rev Clin Exp Hematol. 7 (2003) 22-56.
[30] R. C. Williamson, A. M. Toye, Glycophorin A: Band 3 aid, Blood Cells Mol Dis. 41 (2008) 35-43.
[31] M. L. Meistrich, B. Mohapatra, C. R. Shirley, M. Zhao, Roles of transition nuclear proteins in spermiogenesis, Chromosoma. 111 (2003) 483-488.
[32] J. Lövgren, K. Airas, H. Lilja, Enzymatic action of human glandular kallikrein 2 (hK2). Substrate specificity and regulation by Zn2+ and extracellular protease inhibitors, Eur J Biochem. 262 (1999) 781-789.
[33] J. A. Clements, N. M. Willemsen, S. A. Myers, Y. Dong, The tissue kallikrein family of serine proteases: Functional roles in human disease and potential as clinical biomarkers, Crit Rev Clin Lab Sci. 41 (2004) 265-312.
[34] J. Lövgren, C. Valtonen-Andre, K. Marsal, H. Lilja, A. Lundwall, Measurement of prostate-specific antigen and human glandular kallikrein 2 in different body fluids, J Androl. 20 (1999) 348-355.
[35] S. E. Gill, W. C. Parks, Metalloproteinases and their inhibitors: Regulators of wound healing, Int J Biochem Cell Biol. 40 (2008) 1334-1347.
[36] M. Bauer, D. Patzelt, Identification of menstrual blood by real time RT-PCR: Technical improvements and the practical value of negative test results, Forensic Sci Int. 174 (2008) 55-59.
[37] Y. Yoshiko, J. E. Aubin, Stanniocalcin 1 as a pleiotropic factor in mammals, Peptides. 25 (2004) 1663-1669.
[38] B. H. Yeung, A. Y. Law, C. K. Wong, Evolution and roles of stanniocalcin, Mol Cell Endocrinol. 349 (2012) 272-280.
[39] M. van den Berge, B. Bhoelai, J. Harteveld, A. Matai, T. Sijen, Advancing forensic RNA typing: On non-target secretions, a nasal mucosa marker, a differential co-extraction protocol and the sensitivity of DNA and RNA profiling, Forensic Sci Int Genet. 20 (2016) 119-129.
[40] Sachs A B. Messenger RNA degradation in eukaryotes. Cell. 1993; 74:413-21.
[41] Houseley J, Tollervey D. The many pathways of RNA degradation. Cell. 2009; 136:763-76.
[42] Frazão C, McVey C E, Amblar M, Barbas A, Vonrhein C, Arraiano C M, et al. Unraveling the dynamics of RNA degradation by ribonuclease II and its RNA-bound complex. Nature. 2006; 443:110-4.
[43] van Hoof A, Parker R. Messenger RNA degradation: beginning at the end. Current Biology. 2002; 12:R285-R7.
[44] Christodoulou D C, Gorham J M, Herman D S, Seidman J. Construction of normalized RNA-seq libraries for Next-Generation Sequencing using the crab duplex-specific nuclease. Current Protocols in Molecular Biology. 2011:4.12. 1-4. 1.
[45] Fleige S, Waif V, Huch S, Prgomet C, Sehm J, Pfaffl M W. Comparison of relative mRNA quantification models and the impact of RNA integrity in quantitative real-time RT-PCR. Biotechnology Letters. 2006; 28:1601-13.
[46] Rowley J W, Oler A J, Tolley N D, Hunter B N, Low E N, Nix D A, et al. Genome-wide RNA-seq analysis of human and mouse platelet transcriptomes. Blood. 2011; 118:e101-e11.
[47] Schroeder A, Mueller O, Stocker S, Salowsky R, Leiber M, Gassmann M, et al. The RIN: an RNA integrity number for assigning integrity values to RNA measurements. BMC Molecular Biology. 2006; 7:3.
[48] Auer H, Lyianarachchi S, Newsom D, Klisovic M I. Chipping away at the chip bias: RNA degradation in microarray analysis. Nature Genetics. 2003; 35:292-3.
[49] Fleige S, Pfaffl M W. RNA integrity and the effect on the real-time qRT-PCR performance. Molecular Aspects of Medicine. 2006; 27:126-39.
[50] Romero I G, Pai A A, Tung J, Gilad Y. RNA-seq: Impact of RNA degradation on transcript quantification. BMC Biology. 2014; 12:42.
[51] Antonov J, Goldstein D R, Oberli A, Baltzer A, Pirotta M, Fleischmann A, et al. Reliable gene expression measurements from degraded RNA by quantitative real-time PCR depend on short amplicons and a proper normalization. Laboratory Investigation. 2005; 85:1040-50.
[52] Miyagawa Y, Nishimura H, Tsujimura A, Matsuoka Y, Matsumiya K, Okuyama A, Nishimune Y, Tanaka H. Single-nucelotide polymorphisms and mutation analyses of the TNP1 and TNP2 genes of fertile and infertile human male populations. Journal of Andrology. 2005; 26:779-786.
[53] P. P. Albani, R. Fleming, Novel messenger RNAs for body fluid identification, Science & Justice. (2018) 58:145-152.
[54] D. A. Benson, M. Cavanaugh, K. Clark, I. Karsch-Mizrachi, D. J. Lipman, J. Ostell, et al., GenBank, Nucleic Acids Res. 41 (2013) D36-D42.
[55] R. C. Hardison, Evolution of hemoglobin and its genes, Cold Spring Harb Perspect Med. 2 (2012) a011627.
[56] O. Henegariu, N. A. Heerema, S. R. Dlouhy, G. H. Vance, P. H. Vogt, Multiplex PCR: Critical parameters and step-by-step protocol, BioTechniques. 23 (1997) 504-511.
[57] G. E. Lemack, M. Goldstein, Presence of sperm in the pre-vasectomy reversal semen analysis: Incidence and implications, J Urol. 155 (1996) 167-169.
[58] I. S. Fraser, G. McCarron, R. Markham, T. Resta, Blood and total fluid content of menstrual discharge, Obstet Gynecol. 65 (1985) 194-198.
[59] C. Haas, B. Klesser, A. Kratzer, W. Bär, mRNA profiling for body fluid identification, Forensic Sci Int Genet Supplement Series. 1 (2008) 37-38.
[60] J. Harteveld, A. Lindenbergh, T. Sijen, RNA cell typing and DNA profiling of mixed samples: Can cell types and donors be associated? Sci Justice. 53 (2013) 261-269.
[61] H. Nakanishi, M. Hara, S. Takahashi, A. Takada, K. Saito, Evaluation of forensic examination of extremely aged seminal stains, Leg Med. 16 (2014) 303-307.
[62] A. Lindenbergh, P. Maaskant, T. Sijen, Implementation of RNA profiling in forensic casework, Forensic Sci Int Genet. 7 (2013) 159-166.
[63] Zhao, Shanrong, Baohong, Zhang, Ying Zhang, William Gordon, Sarah Du, Theresa Paradis, Michael Vincent, and David von Schack. “Bioinformatics for RNA-Seq Data Analysis.” BIOINFORMATICS-UPDATED FEATURES AND APPLICATIONS (2016): 125.
[64] Chomczynski, Piotr, and Nicoletta Sacchi. The single-step method of RNA isolation by acid guanidinium thiocyanate-phenol-chloroform extraction: twenty-something years on. Nature protocols 1(2) (2006): 581-585.
[65] Berensmeier, Sonja. “Magnetic particles for the separation and purification of nucleic acids.” Applied microbiology and biotechnology 73(3) (2006): 495-504.
[66] Matson, R. S. (2008). Microarray Methods and Protocols. Boca Raton, Fla.: CRC. pp. 7-29. ISBN 1420046659.
[67] Kumar, A. (2006). Genetic Engineering. New York: Nova Science Publishers. pp. 101-102. ISBN 159454753X).
[68] Rio, D. C., Ares, M., Hannon, G. J., & Nilsen, T. W. Purification of RNA using TRIzol (TRI reagent). Cold Spring Harbor Protocols, (2010), pdb-prot5439.
[69] Mardis, E. R. (2008). The impact of next-generation sequencing technology on genetics. Trends in genetics, 24(3), 133-141.
[70] Metzker, M. L. (2010). Sequencing technologies—the next generation. Nature Reviews Genetics, 11(1), 31-46.
[71] Reis-Filho, J. S. (2009). Next-generation sequencing. Breast Cancer Res, 11(Suppl 3), S12.
[72] Schuster, S. C. (2008). Next-generation sequencing transforms today's biology. Nature methods, 5(1), 16-18.
[73] Mutz, K. O., Heilkenbrinker, A., Lonne, M., Walter, J. G., & Stahl, F. (2013). Transcriptome analysis using next-generation sequencing. Current opinion in biotechnology, 24(1), 22-30.
[74] Fuller, C. W., Middendorf, L. R., Benner, S. A., Church, G. M., Harris, T., Huang, X., Jovanovich, S. B., Nelson, J. R., Schloss, J. A., Schwartz, D. C, & Vezenov, D. V. (2009). The challenges of sequencing by synthesis. Nature biotechnology, 27(11), 1013-1023.
[75] Patel, R. K., & Jain, M. (2012). NGS Q C Toolkit: a toolkit for quality control of next generation sequencing data. PloS one, 7(2), e30619.
[76] Trapnell, C., Pachter, L., & Salzberg, S. L. (2009). TopHat: discovering splice junctions with RNA-Seq. Bioinformatics, 25(9), 1105-1111.
[77] Mullis, K. B., & Gibbs, F. F. R. (1994). Richard A. Morgan and W. French Anderson. The Polymerase chain reaction, 357.
[78] Davies, M. J., Shah, A., & Bruce, I. J. (2000). Synthesis of fluorescently labelled oligonucleotides and nucleic acids. Chemical Society Reviews, 29(2), 97-107.
[79] Proudnikov, D., & Mirzabekov, A. (1996). Chemical methods of DNA and RNA fluorescent labeling. Nucleic acids research, 24(22), 4535-4542.
[80] Kutyavin, I. V., Afonina, I. A., Mills, A., Gorn, V. V., Lukhtanov, E. A., Belousov, E. S., Singer, M. J., Walburger, D. K., Lokhov, S. G., Gall, A. A., Dempcy, R., Reed, M. W., Meyer, R. B. & Hedgpeth, J. (2000). 3′-minor groove binder-DNA probes increase sequence specificity at PCR extension temperatures. Nucleic Acids Research, 28(2), 655-661.
[81] Pon, R. T. (1991). A long chain biotin phosphoramidite reagent for the automated synthesis of 5′-biotinylated oligonucleotides. Tetrahedron letters, 32(14), 1715-1718.
[82] Agrawal, S., Christodoulou, C., & Gait, M. J. (1986). Efficient methods for attaching non-radioactive labels to the 5′ ends of synthetic oligodeoxyribonucleotides. Nucleic acids research, 14(15), 6227-6245.
[83] Sambrook et al., Eds, 1987, Molecular Cloning, A Laboratory Manual, 2nd Ed. Cold Spring Harbor Press.
[84] Tyagi, S., & Kramer, F. R. (1996). Molecular beacons: probes that fluoresce upon hybridization. Nature biotechnology, (14), 303-8.
[85] R Carters, R., Ferguson, J., Gaut, R., Ravetto, P., Thelwell, N., & Whitcombe, D. (2008). Design and use of scorpions fluorescent signaling molecules. In Molecular beacons: Signalling nucleic acid probes, methods, and protocols (pp. 99-115). Humana Press.
[86] Eisel, D.; Grünewald-Janho, S.; Krushen, B., ed. (2002). DIG Application Manual for Nonradioactive in situ Hybridization (3rd ed.). Penzberg: Roche Diagnostics.
[87] Simmons, D. M., Arriza, J. L., & Swanson, L. W. (1989). A complete protocol for in situ hybridization of messenger RNAs in brain and other tissues with radio-labeled single-stranded RNA probes. Journal of Histotechnology, 12(3), 169-181.
[88] Bowden, A., Fleming, R., & Harbison, S. (2011). A method for DNA and RNA co-extraction for use on forensic samples using the Promega DNA IQ™ system. Forensic Science International: Genetics, 5(1), 64-68).
[89] Tatiana A. Tatusova, Thomas L. Madden (1999), “Blast 2 sequences—a new tool for comparing protein and nucleotide sequences”, FEMS Microbiol Lett. 174:247-250.
[90] Needleman, S. B. and Wunsch, C. D. (1970) J. Mol. Biol. 48, 443-453.
[91] Rice,P. Longden,l. and Bleasby, A. EMBOSS: The European Molecular Biology Open Software Suite, Trends in Genetics June 2000, vol 16, No 6. pp. 276-277.
[92] Huang, X. (1994) On Global Sequence Alignment. Computer Applications in the Biosciences 10, 227-235.
[93] Thompson et al., 1994, Nucleic Acids Research 24, 4876-4882.
[94] Bauer, D. Patzelt, Protamine mRNA as molecular marker for spermatozoa in semen stains, Int J Legal Med. 117 (2003) 175-179.

Hemoglobin delta (HBD)
SEQ ID NO: 1
AGGGCAAGTT AAGGGAATAG TGGAATGAAG GTTCATTTTT CATTCTCACA AACTAATGAA
ACCCTGCTTA TCTTAAACCA ACCTGCTCAC TGGAGCAGGG AGGACAGGAC CAGCATAAAA
GGCAGGGCAG AGTCGACTGT TGCTTACACT TTCTTCTGAC ATAACAGTGT TCACTAGCAA
CCTCAAACAG ACACCATGGT GCATCTGACT CCTGAGGAGA AGACTGCTGT CAATGCCCTG
TGGGGCAAAG TGAACGTGGA TGCAGTTGGT GGTGAGGCCC TGGGCAGATT ACTGGTGGTC
TACCCTTGGA CCCAGAGGTT CTTTGAGTCC TTTGGGGATC TGTCCTCTCC TGATGCTGTT
ATGGGCAACC CTAAGGTGAA GGCTCATGGC AAGAAGGTGC TAGGTGCCTT TAGTGATGGC
CTGGCTCACC TGGACAACCT CAAGGGCACT TTTTCTCAGC TGAGTGAGCT GCACTGTGAC
AAGCTGCACG TGGATCCTGA GAACTTCAGG CTCTTGGGCA ATGTGCTGGT GTGTGTGCTG
GCCCGCAACT TTGGCAAGGA ATTCACCCCA CAAATGCAGG CTGCCTATCA GAAGGTGGTG
GCTGGTGTGG CTAATGCCCT GGCTCACAAG TACCATTGAG ATCCTGGACT GTTTCCTGAT
AACCATAAGA AGACCCTATT TCCCTAGATT CTATTTTCTG AACTTGGGAA CACAATGCCT
ACTTCAAGGG TATGGCTTCT GCCTAATAAA GAATGTTCAG CTCAACTTCC TGAT
Solute carrier family 4 (anion exchanger), member 1 (Diego blood group)
(SLC4A1)
SEQ ID NO: 2
GAACGAGTGG GAACGTAGCT GGTCGCAGAG GGCACCAGCG GCTGCAGGAC TTCACCAAGG
GACCCTGAGG CTCGTGAGCA GGGACCCGCG GTGCGGGTTA TGCTGGGGGC TCAGATCACC
GTAGACAACT GGACACTCAG GACCACGCCA TGGAGGAGCT GCAGGATGAT TATGAAGACA
TGATGGAGGA GAATCTGGAG CAGGAGGAAT ATGAAGACCC AGACATCCCC GAGTCCCAGA
TGGAGGAGCC GGCAGCTCAC GACACCGAGG CAACAGCCAC AGACTACCAC ACCACATCAC
ACCCGGGTAC CCACAAGGTC TATGTGGAGC TGCAGGAGCT GGTGATGGAC GAAAAGAACC
AGGAGCTGAG ATGGATGGAG GCGGCGCGCT GGGTGCAACT GGAGGAGAAC CTGGGGGAGA
ATGGGGCCTG GGGCCGCCCG CACCTCTCTC ACCTCACCTT CTGGAGCCTC CTAGAGCTGC
GTAGAGTCTT CACCAAGGGT ACTGTCCTCC TAGACCTGCA AGAGACCTCC CTGGCTGGAG
TGGCCAACCA ACTGCTAGAC AGGTTTATCT TTGAAGACCA GATCCGGCCT CAGGACCGAG
AGGAGCTGCT CCGGGCCCTG CTGCTTAAAC ACAGCCACGC TGGAGAGCTG GAGGCCCTGG
GGGGTGTGAA GCCTGCAGTC CTGACACGCT CTGGGGATCC TTCACAGCCT CTGCTCCCCC
AACACTCCTC ACTGGAGACA CAGCTCTTCT GTGAGCAGGG AGATGGGGGC ACAGAAGGGC
ACTCACCATC TGGAATTCTG GAAAAGATTC CCCCGGATTC AGAGGCCACG TTGGTGCTAG
TGGGCCGCGC CGACTTCCTG GAGCAGCCGG TGCTGGGCTT CGTGAGGCTG CAGGAGGCAG
CGGAGCTGGA GGCGGTGGAG CTGCCGGTGC CTATACGCTT CCTCTTTGTG TTGCTGGGAC
CTGAGGCCCC CCACATCGAT TACACCCAGC TTGGCCGGGC TGCTGCCACC CTCATGTCAG
AGAGGGTGTT CCGCATAGAT GCCTACATGG CTCAGAGCCG AGGGGAGCTG CTGCACTCCC
TAGAGGGCTT CCTGGACTGC AGCCTAGTGC TGCCTCCCAC CGATGCCCCC TCCGAGCAGG
CACTGCTCAG TCTGGTGCCT GTGCAGAGGG AGCTACTTCG AAGGCGCTAT CAGTCCAGCC
CTGCCAAGCC AGACTCCAGC TTCTACAAGG GCCTAGACTT AAATGGGGGC CCAGATGACC
CTCTGCAGCA GACAGGCCAG CTCTTCGGGG GCCTGGTGCG TGATATCCGG CGCCGCTACC
CCTATTACCT GAGTGACATC ACAGATGCAT TCAGCCCCCA GGTCCTGGCT GCCGTCATCT
TCATCTACTT TGCTGCACTG TCACCCGCCA TCACCTTCGG CGGCCTCCTG GGAGAAAAGA
CCCGGAACCA GATGGGAGTG TCGGAGCTGC TGATCTCCAC TGCAGTGCAG GGCATTCTCT
TCGCCCTGCT GGGGGCTCAG CCCCTGCTTG TGGTCGGCTT CTCAGGACCC CTGCTGGTGT
TTGAGGAAGC CTTCTTCTCG TTCTGCGAGA CCAACGGTCT AGAGTACATC GTGGGCCGCG
TGTGGATCGG CTTCTGGCTC ATCCTGCTGG TGGTGTTGGT GGTGGCCTTC GAGGGTAGCT
TCCTGGTCCG CTTCATCTCC CGCTATACCC AGGAGATCTT CTCCTTCCTC ATTTCCCTCA
TCTTCATCTA TGAGACTTTC TCCAAGCTGA TCAAGATCTT CCAGGACCAC CCACTACAGA
AGACTTATAA CTACAACGTG TTGATGGTGC CCAAACCTCA GGGCCCCCTG CCCAACACAG
CCCTCCTCTC CCTTGTGCTC ATGGCCGGTA CCTTCTTCTT TGCCATGATG CTGCGCAAGT
TCAAGAACAG CTCCTATTTC CCTGGCAAGC TGCGTCGGGT CATCGGGGAC TTCGGGGTCC
CCATCTCCAT CCTGATCATG GTCCTGGTGG ATTTCTTCAT TCAGGATACC TACACCCAGA
AACTCTCGGT GCCTGATGGC TTCAAGGTGT CCAACTCCTC AGCCCGGGGC TGGGTCATCC
ACCCACTGGG CTTGCGTTCC GAGTTTCCCA TCTGGATGAT GTTTGCCTCC GCCCTGCCTG
CTCTGCTGGT CTTCATCCTC ATATTCCTGG AGTCTCAGAT CACCACGCTG ATTGTCAGCA
AACCTGAGCG CAAGATGGTC AAGGGCTCCG GCTTCCACCT GGACCTGCTG CTGGTAGTAG
GCATGGGTGG GGTGGCCGCC CTCTTTGGGA TGCCCTGGCT CAGTGCCACC ACCGTGCGTT
CCGTCACCCA TGCCAACGCC CTCACTGTCA TGGGCAAAGC CAGCACCCCA GGGGCTGCAG
CCCAGATCCA GGAGGTCAAA GAGCAGCGGA TCAGTGGACT CCTGGTCGCT GTGCTTGTGG
GCCTGTCCAT CCTCATGGAG CCCATCCTGT CCCGCATCCC CCTGGCTGTA CTGTTTGGCA
TCTTCCTCTA CATGGGGGTC ACGTCGCTCA GCGGCATCCA GCTCTTTGAC CGCATCTTGC
TTCTGTTCAA GCCACCCAAG TATCACCCAG ATGTGCCCTA CGTCAAGCGG GTGAAGACCT
GGCGCATGCA CTTATTCACG GGCATCCAGA TCATCTGCCT GGCAGTGCTG TGGGTGGTGA
AGTCCACGCC GGCCTCCCTG GCCCTGCCCT TCGTCCTCAT CCTCACTGTG CCGCTGCGGC
GCGTCCTGCT GCCGCTCATC TTCAGGAACG TGGAGCTTCA GTGTCTGGAT GCTGATGATG
CCAAGGCAAC CTTTGATGAG GAGGAAGGTC GGGATGAATA CGACGAAGTG GCCATGCCTG
TGTGAGGGGC GGGCCCAGGC CCTAGACCCT CCCCCACCAT TCCACATCCC CACCTTCCAA
GGAAAAGCAG AAGTTCATGG GCACCTCATG GACTCCAGGA TCCTCCTGGA GCAGCAGCTG
AGGCCCCAGG GCTGTGGGTG GGGAAGGAAG GCGTGTCCAG GAGACCTTCC ACAAAGGGTA
GCCTGGCTTT TCTGGCTGGG GATGGCCGAT GGGGCCCACA TTAGGGGGTT TGTTGCACAG
TCCCTCCTGT TGCCACACTT TCACTGGGGA TCCCGTGCTG GAAGACTTAG ATCTGAGCCC
TCCCTCTTCC CAGCACAGGC AGGGGTAGAA GCAAAGGCAG GAGGTGGGTG AGCGGGTGGG
GTGCTTGCTG TGTGACCTTG GGCAAGTCCC TTGACCTTTC CAGCCTATAT TTCCTCTTCT
GTAAAATGGG TATATTGATG ATAATACCCA CATTACAGGA TGGTTACTGA GGACCAAAGA
TACATGTAAA ATAGGGCTTT GTAAACTCCA CAGGGACTGT TCTATAGCAG TCATCATTTG
TCTTTGAACG TACCCAAGGT CACATAGCTG GGATTTGAAC TGAGCCGTGC AGCTGGGATT
TGAACCAGGC CTTCTGATTT CAAGGTCCGA GCTCTGTCCT CTGTCAGTCA TGCGTCCACT
TTCCCTTCCC CTGTGACTCC TCCCTTCCCC ACTCTGCTCC CAGCCCCTAC CTTGAGACCC
TCTTCTCTGG GCCCAGAGAG AGGCGTCCTG GTGAGGACAA GGTACAGGCA AGGATGATCC
AGGGATTGGG CCTGGGACTC AGGCCTCCTA AGTGTTTGGT TCCTCCCTCC AAACACTCAT
TAGTTCACTC ATTCATTCAT TCCACAAACA TTTACTGAGG GCCCCGGAAT CAGTGGACTC
CGAGGGGACT GAGACAAGCC CTGCCCTGGG GTGGGGGTGG GGGGCAAGGT ACAGTTGATT
CTACATTTGG ATAGGGAGTG GGGGAGGGTG GGAAGGTAGG GGCGGGAGAG TGAGGGGGTT
TGTAATTTAT TAATTGCGTA TTTTCTAAGA GTTTTCAACA TAGTTTGGCT TCACACACAA
CTTCAGGCCC CTCATTTGAG AGCCATTATC CTCAACTCCA TCTAAACTGA ATCTTGGGGA
GAACCCAGAT CTGACCAATT GGGGTAGGAG ACAGCAGGCT CTCCAAGAAC ATGGGCAAAT
TTATTTTTTT ATAAAACAAA AAGATAAAAA GAGTTGAAAG ACGTGAAAGT GGTGAGAGAT
GGAGGAAACA GAATCAGGAA GTGGTAGAAA AGAGAGGAGG TGGCTGGGCG CAGTGGCTCA
CGTTTGTAAT CCCAGCACTT TGGGAGGCCA AGTTGGGCGG ATCATTTGAG GTCAGGAGTT
TGAGACCAGC CTGGCCAACA TGGTGAAACC CCGTTACTAC TAAAAATACA AAAATTAGCT
GGGTGTCTCG TGGCAGGCAC CTGTAATCCC AGCTACTTAG AAGGCTGAGG CAAGAGAATC
ACCTGAACCC AGGAGGTGGA GGTTGCAGTG AGCCAAGATT GCACCACTGC ACTCCAGCCT
GGGCAACAGA GCGAGACCCT GTCTCAAAAA AAAAAAAAAA AAAAAAAAAA AAACGGAAGG
AAACATCAGC CTTGGGGGCC ACAGACTCAA CATGTGTGTG TGGTGGGGTT CCAGCCCAAC
ATAGAGTAAC ATTATTTGTA CCTCCCAGGC TAGCTCAGTC CATGGGAGGC TCTCCTGTCC
CTGAAAGCTG ACACCCACCT TTCACCACTT CGCCCATGCT ACAGTTCAGT TTCCTCGTCT
GTAAAATGGG GATGATAATG GTACCTACCT TGCAGTGTTG TTATAAGGAT TAAAGGAGAC
AGTGCAAGAA AAGGCCTTGG TTGGTGAAGA GCCCAACCTC GGAGGGGAGC TGCTGGGATC
CTCCTTATCT TGACTGGGAT GTCCCTGTCT CCCCCTCCCC TTGCTCCTTG AACATGGCCA
AGGAAAGTGA AAAACAAAAA TTATTCACTC TGCTAGCACC CTTCCCCTTG ATGCCTGGGA
ATAGGTTTTG CCAATAAACG TATCTGTGTT GGA
Glycophorin A (MNS blood group) (GYPA)
SEQ ID NO: 3
AAAATGCCTC CCCTGCCTAT CAGCTGATGA TGGCCGCAGG AAGGTGGGCC TGGAAGATAA
CAGCTAGCAG GCTAAGGTCA GACACTGACA CTTGCAGTTG TCTTTGGTAG TTTTTTTGCA
CTAACTTCAG GAACCAGCTC ATGATCTCAG GATGTATGGA AAAATAATCT TTGTATTACT
ATTGTCAGAA ATTGTGAGCA TATCAGCATT AAGTACCACT GAGGTGGCAA TGCACACTTC
AACTTCTTCT TCAGTCACAA AGAGTTACAT CTCATCACAG ACAAATGATA CGCACAAACG
GGACACATAT GCAGCCACTC CTAGAGCTCA TGAAGTTTCA GAAATTTCTG TTAGAACTGT
TTACCCTCCA GAAGAGGAAA CCGAGATAAC ACTCATTATT TTTGGGGTGA TGGCTGGTGT
TATTGGAACG ATCCTCTTAA TTTCTTACGG TATTCGCCGA CTGATAAAGA AAAGCCCATC
TGATGTAAAA CCTCTCCCCT CACCTGACAC AGACGTGCCT TTAAGTTCTG TTGAAATAGA
AAATCCAGAG ACAAGTGATC AATGAGAATC TGTTCACCAA ACCAAATGTG GAAAGAACAC
AAAGAAGACA TAAGACTTCA GTCAAGTGAA AAATTAACAT GTGGACTGGA CACTCCAATA
AATTATATAC CTGCCTAAGT TGTACAATTT CAGAATGCAA TTTTCATTAT AATGAGTTCC
AGTGACTCAA TGATGGGGAA AAAAATCTCT GCTCATTAAT ATTTCAAGAT AAAGAACAAA
TGTTTCCTTG AATGCTTGCT TTTGTGTGTT AGCATAATTT TTAGAATTGT TTGAGAATTC
TGATCCAAAA CTTTAGTTGA ATTCATCTAC GTTTGTTTAA TATTAACTTA ACCTATTCTA
TTGTATTATA ATGATGATTC TGTCAAATGA AAGGCTTGAA ATACCTAGAT GAAGTTTAGA
TTTTCTTCCT ATTGTAAACT TTTGAGTCTG GTTTCATTGT TTTAAATAAA TTAAGGGGAC
ACTAAAGTCC TATCATTCAT TTCCTTCATT GCTGAACAGG CAAGATATAA TATTACATGA
ATGATTACTA TATTTTGTTC ACACTAATAA AGCTTATGCT CAGAAATGCC ATACACACAC
ACACACACAC ACACAAACAC ACACATTTAT CATTTAATGC ATAAATCAAC ACAAAAGGTT
TTCCCATTAA TATGAAATAT TACATATATA TAAGTGCCAT ATTTAAAATA ATTTGTCTAA
CAGTAGAACT GTGTCGGAGC ACTCACTGAA GCTTGCATTC CACTGAAAGA GTTATTTGTG
TAAGTAGAGT ATCCGGAGAA GGAAAAGAAC TTACGACCTT TCTTTATAAC AGAAACTCAA
CTCTAAATTC AACAAGATGT GCAAACCGGA CATGCAGGTG AATATTTTAA TAGGTTACTA
TAAGGTTCTC AATTAAATTC TTTAATCTGT CCAGTCCCAG TTTCTCTTAT TAATAAAACT
TTGGAAATTG CTTTAAACCA TTTAAAGGAA ATTTCTAGAT ATAGAAACTA AGGACTGTGA
CTATACAGCT GTCACTCATT TGTAGTAAAA CTTAAAAAGC AAAAACAAAA AACAAAAAAG
ACCTTCCTGT GATACTTTAT TTCCGAACTA ATAAAAATCT ATATGACTTT TTATTATTGT
GTGATAACCA AGTAAATGTT TTCTATTTTG CATATTTTCA GGCATGGTAA CAGAAATTTA
CCTTTTAATA AATTAAAAAA TCTAAATTTT AACCTACTTG TATGTTCGGA GAGTGTTTTT
GTACTATATT GACTACTTAA AATAGAGAAT GAGACTAAGA AGGGAACATT TCTGTTGATA
CATGTTTTTT AAAAGTAATT TTAAGAGCAT TATTAGGTTA ATTAATCCAA TTAATGACCC
AAATGCCAAG GTAATTTTAA ATTTACATTT TTAATAAAAG CAACATGTTG AAACAAGAGA
GGGTGAGATT AACCTTTTTG CTAAAGTAAT TTACAAGTCA AAGACAGGAA GAGATCAGAG
TGAATGTGCC TTCTTAACCA GAGCTACAGA ATTTAGTGAA TAATTAAAGT ACAAACTGCT
TTGACCTCCT TGAACTTTTC CAAGCAATTT CTCTGTACTT CTATATATGA ATGTCTTAGC
CAATTTTCTG CTACTATAAC AGAATACGAC AGACTGGGTA ATTTAAAAAG AAAAGAAATT
TATTTTCTTC CTAGTTCTGG AGGCTGGGAA GGCGAAGGGC ATGGCACTGA CATCTGCCTT
GTAACTGATG AGAACCTTCT TACTGCATGA TAACAAAGCA GCAAGGCAAG CAAAAGCGTA
AGATGAAGAG AGAGGAAATG AAGCCAAACA CATCCTTTCA TCAGAAGCCC ATTCCCTCTA
TAAGGCGTTA TTACATTTAT GAGAATGGAG TCCTCATGAC CTAATCGTGA CCTTAAAGGC
CCCTCCCAAC ACTGTTACAA TGGCAATTAA ATTTCAACAA AGGTTCCAGA GGTGACATTC
GAATCAGCAA TGAAATTTTC ATAGTTAAAT TTGGTATTCG TGGGGGAAGA AATGACCATT
TCCCTTGTAT TTTTATAATT AAATCAGCAA AATATTGTAA TAAAGAAATC TTTCCTGTGA
AGATACCATG ACCCCAAAAA AAAAAA
Follicular dendritic cell secreted protein (FDCSP)
SEQ ID NO: 4
CTCCATTCCA TTATACCTTT GAGTATATAA AACAGCTACA ATATTCCAGG GCCAGTCACT
TGCCATTTCT CATAACAGCG TCAGAGAGAA AGAACTGACT GAAACGTTTG AGATGAAGAA
AGTTCTCCTC CTGATCACAG CCATCTTGGC AGTGGCTGTT GGTTTCCCAG TCTCTCAAGA
CCAGGAACGA GAAAAAAGAA GTATCAGTGA CAGCGATGAA TTAGCTTCAG GGTTTTTTGT
GTTCCCTTAC CCATATCCAT TTCGCCCACT TCCACCAATT CCATTTCCAA GATTTCCATG
GTTTAGACGT AATTTTCCTA TTCCAATACC TGAATCTGCC CCTACAACTC CCCTTCCTAG
CGAAAAGTAA ACAAGAAGGA AAAGTCACGA TAAACCTGGT CACCTGAAAT TGAAATTGAG
CCACTTCCTT GAAGAATCAA AATTCCTGTT AATAAAAGAA AAACAAATGT AATTGAAATA
GCACACAGCA TTCTCTAGTC AATATCTTTA GTGATCTTCT TTAATAAACT TGAAAGCAAA
GATTTTGGTT TCTTAATTTC CACAAAAAAA AAA
Histatin 3 (HTN3)
SEQ ID NO: 5
GGGAGATTTC AACGTGTTTA AATACATCAG CCATCTAGGA AAGGACATCT CTTGAGACTT
CACTTCAGCT TCACTGACTT CTGGATTCTC CTCTTGAGTA AAAGGACTCA GCCAACTATG
AAGTTTTTTG TTTTTGCTTT AATCTTGGCT CTCATGCTTT CCATGACTGG AGCTGATTCA
CATGCAAAGA GACATCATGG GTATAAAAGA AAATTCCATG AAAAGCATCA TTCACATCGA
GGCTATAGAT CAAATTATCT GTATGACAAT TGATATCTTC AGTAATCACG GGGCATGATT
ATGGAGGTTT GACTGGCAAA TTCGCTTTGG ACTCGTGTAT TCTCATTTGT CATACCGCAT
CACACTACCA CTGCTTTTTG AAGAATTATC ATAAGGCAAT GCAGAATAAA AGAAATACCA
TGATTTAGTG AATTCTGTGT TTCAGGATAC TTCCCTTCCT AATTATCATT TGATTAGATA
CTTGCAATTT AAATGTTAAG CTGTTTTCAC TGCTGTTTCT GAGTAATAGA AATTCATTCC
TCTCCAAAAG CAATAAAATT CAAGCACATT ATTATGTGAA AAAAAAAAAA AAAAAAAAAA A
(polynucleotide, statherin (STATH)
SEQ ID NO: 6
GAGTGTTTAA ATACATTGGC CCTCTAGGGT AGCACATCAT CTCTTGAAGC TTCACTTCAA
CTTCACTACT TCTGTAGTCT CATCTTGAGT AAAAGAGAAC CCAGCCAACT ATGAAGTTCC
TTGTCTTTGC CTTCATCTTG GCTCTCATGG TTTCCATGAT TGGAGCTGAT TCATCTGAAG
AGTATGGGTA TGGCCCTTAT CAGCCAGTTC CAGAACAACC ACTATACCCA CAACCATACC
AACCACAATA CCAACAATAT ACCTTTTAAT ATCATCAGTA ACTGCAGGAC ATGATTATTG
AGGCTTGATT GGCAAATACG ACTTCTACAT CCATATTCTC ATCTTTCATA CCATATCACA
CTACTACCAC TTTTTGAAGA ATCATCAAAG AGCAATGCAA ATGAAAAACA CTATAATTTA
CTGTATACTC TTTGTTTCAG GATACTTGCC TTTTCAATTG TCACTTGATG ATATAATTGC
AATTTAAACT GTTAAGCTGT GTTCAGTACT GTTTCTGAAT AATAGAAATC ACTTCTCTAA
AAGCAATAAA TTTCAAGCAC ATTTTTACAT AAAAAAAA
Protamine 1 (PRM1)
SEQ ID NO: 7
GACTCACAGC CCACAGAGTT CCACCTGCTC ACAGGTTGGC TGGCTCAGCC AAGGTGGTGC
CCTGCTCTGA GCATTCAGGC CAAGCCCATC CTGCACCATG GCCAGGTACA GATGCTGTCG
CAGCCAGAGC CGGAGCAGAT ATTACCGCCA GAGACAAAGA AGTCGCAGAC GAAGGAGGCG
GAGCTGCCAG ACACGGAGGA GAGCCATGAG GTGCTGCCGC CCCAGGTACA GACCGCGATG
TAGAAGACAC TAATTGCACA AAATAGCACA TCCACCAAAC TCCTGCCTGA GAATGTTACC
AGACTTCAAG ATCCTCTTGC CACATCTTGA AAATGCCACC ATCCAATAAA AATCAGGAGC
CTGCTAAGGA ACAATGCCGC CTGTCAATAA ATGTTGAAAA GTCATCCCAA AAAAAAAAAA
AAAAAA
Transition protein 1 (TNP1)
SEQ ID NO: 8
GCCCCTCATT TTGGCAGAAC TTACCATGTC GACCAGCCGC AAATTAAAGA GTCATGGCAT
GAGGAGGAGC AAGAGCCGAT CTCCTCACAA GGGAGTCAAG AGAGGTGGCA GCAAAAGAAA
ATACCGTAAG GGCAACCTGA AAAGTAGGAA ACGGGGCGAT GACGCCAATC GCAATTACCG
CTCCCACTTG TGAGCCCCCA GCGGGCTCTG CCCTGGTGCG CTTCACACAG CACCAAGCAG
CAACAAGAAC AGCAGAAGGG GAACTGCCAA GGAGACCTGA TGTTAGATCA AAGCCAGAGA
GGAGCCTATG GAATGTGGAT CAAATGCCAG TTGTGACGAA ATGAGGAATG TATATGTTGG
CTGTTTTTCC CCAACATCTC AATAAAACTT TGAAAGCAGA AAAAAAAAAA AAAAA
Protamine 2 (PRM2)
SEQ ID NO: 9
AGACCAGACC AACAGTAACA CCAAGGGCAG GTGGGCAGGC CTCCGCCCTC CTCCCCTACT
CCAGGGCCCA CTGCAGCCTC AGCCCAGGAG CCACCAGATC TCCCAACACC ATGGTCCGAT
ACCGCGTGAG GAGCCTGAGC GAACGCTCGC ACGAGGTGTA CAGGCAGCAG TTGCATGGGC
AAGAGCAAGG ACACCACGGC CAAGAGGAGC AAGGGCTGAG CCCGGAGCAC GTCGAGGTCT
ACGAGAGGAC CCATGGCCAG TCTCACTATA GGCGCAGACA CTGCTCTCGA AGGAGGCTGC
ACCGGATCCA CAGGCGGCAG CATCGCTCCT GCAGAAGGCG CAAAAGACGC TCCTGCAGGC
ACCGGAGGAG GCATCGCAGA GAGTCCCTAG GTGACCCCCT CAACCAGAAC TTTCTTTCCC
AAAAGGCTGC AGAACCAGGA AGAGAACATG CAGAAGGCAC TAAGCTTCCT GGGCCCCTCA
CCCCCAGCTG GAAATTAAGA AAAAGTCGCC CGAAACACCA AGTGAGGCCA TAGCAATTCC
CCTACATCAA ATGCTCAAGC CCCCAGCTGG AAGTTAAGAG AAAGTCACCT GCCCAAGAAA
CACCGAGTGA GGCCATAGCA ACTCCCCTAC ATCAAATGCT CAAGCCCTGA GTTGCCGCCG
AGAAGCCCAC AAGATCTGAG TGAAATGAGC AAAAGTCACC TGCCCAATAA AGCTTGACAA
GACACTC
Kallikrein related peptidase 2 (KLK2)
SEQ ID NO: 10
AGCCCCAAAC TCACCACCTG GCCGTGGACA CCTGTGTCAG CATGTGGGAC CTGGTTCTCT
CCATCGCCTT GTCTGTGGGG TGCACTGGTG CCGTGCCCCT CATCCAGTCT CGGATTGTGG
GAGGCTGGGA GTGTGAGAAG CATTCCCAAC CCTGGCAGGT GGCTGTGTAC AGTCATGGAT
GGGCACACTG TGGGGGTGTC CTGGTGCACC CCCAGTGGGT GCTCACAGCT GCCCATTGCC
TAAAGAAGAA TAGCCAGGTC TGGCTGGGTC GGCACAACCT GTTTGAGCCT GAAGACACAG
GCCAGAGGGT CCCTGTCAGC CACAGCTTCC CACACCCGCT CTACAATATG AGCCTTCTGA
AGCATCAAAG CCTTAGACCA GATGAAGACT CCAGCCATGA CCTCATGCTG CTCCGCCTGT
CAGAGCCTGC CAAGATCACA GATGTTGTGA AGGTCCTGGG CCTGCCCACC CAGGAGCCAG
CACTGGGGAC CACCTGCTAC GCCTCAGGCT GGGGCAGCAT CGAACCAGAG GAGTTCTTGC
GCCCCAGGAG TCTTCAGTGT GTGAGCCTCC ATCTCCTGTC CAATGACATG TGTGCTAGAG
CTTACTCTGA GAAGGTGACA GAGTTCATGT TGTGTGCTGG GCTCTGGACA GGTGGTAAAG
ACACTTGTGG GGTGAGTCAT CCCTACTCCC AACATCTGGA GGGGAAAGGG TGATTCTGGG
GGTCCACTTG TCTGTAATGG TGTGCTTCAA GGTATCACAT CATGGGGCCC TGAGCCATGT
GCCCTGCCTG AAAAGCCTGC TGTGTACACC AAGGTGGTGC ATTACCGGAA GTGGATCAAG
GACACCATCG CAGCCAACCC CTGAGTGCCC CTGTCCCACC CCTACCTCTA GTAAATTTAA
GTCCACCTCA CGTTCTGGCA TCACTTGGCC TTTCTGGATG CTGGACACCT GAAGCTTGGA
ACTCACCTGG CCGAAGCTCG AGCCTCCTGA GTCCTACTGA CCTGTGCTTT CTGGTGTGGA
GTCCAGGGCT GCTAGGAAAA GGAATGGGCA GACACAGGTG TATGCCAATG TTTCTGAAAT
GGGTATAATT TCGTCCTCTC CTTCGGAACA CTGGCTGTCT CTGAAGACTT CTCGCTCAGT
TTCAGTGAGG ACACACACAA AGACGTGGGT GACCATGTTG TTTGTGGGGT GCAGAGATGG
GAGGGGTGGG GCCCACCCTG GAAGAGTGGA CAGTGACACA AGGTGGACAC TCTCTACAGA
TCACTGAGGA TAAGCTGGAG CCACAATGCA TGAGGCACAC ACACAGCAAG GATGACGCTG
TAAACATAGC CCACGCTGTC CTGGGGGCAC TGGGAAGCCT AGATAAGGCC GTGAGCAGAA
AGAAGGGGAG GATCCTCCTA TGTTGTTGAA GGAGGGACTA GGGGGAGAAA CTGAAAGCTG
ATTAATTACA GGAGGTTTGT TCAGGTCCCC CAAACCACCG TCAGATTTGA TGATTTCCTA
GCAGGACTTA CAGAAATAAA GAGCTATCAT GCTGTGGTTT ATTATGGTTT GTTACATTGA
TAGGATACAT ACTGAAATCA GCAAACAAAA CAGATGTATA GATTAGAGTG TGGAGAAAAC
AGAGGAAAAC TTGCAGTTAC GAAGACTGGC AACTTGGCTT TACTAAGTTT TCAGACTGGC
AGGAAGTCAA ACCTATTAGG CTGAGGACCT TGTGGAGTGT AGCTGATCCA GCTGATAGAG
GAACTAGCCA GGTGGGGGCC TTTCCCTTTG GATGGGGGGC ATATCTGACA GTTATTCTCT
CCAAGTGGAG ACTTACGGAC AGCATATAAT TCTCCCTGCA AGGATGTATG ATAATATGTA
CAAAGTAATT CCAACTGAGG AAGCTCACCT GATCCTTAGT GTCCAGGGTT TTTACTGGGG
GTCTGTAGGA CGAGTATGGA GTACTTGAAT AATTGACCTG AAGTCCTCAG ACCTGAGGTT
CCCTAGAGTT CAAACAGATA CAGCATGGTC CAGAGTCCCA GATGTACAAA AACAGGGATT
CATCACAAAT CCCATCTTTA GCATGAAGGG TCTGGCATGG CCCAAGGCCC CAAGTATATC
AAGGCACTTG GGCAGAACAT GCCAAGGAAT CAAATGTCAT CTCCCAGGAG TTATTCAAGG
GTGAGCCCTT TACTTGGGAT GTACAGGCTT TGAGCAGTGC AGGGCTGCTG AGTCAACCTT
TTATTGTACA GGGGATGAGG GAAAGGGAGA GGATGAGGAA GCCCCCCTGG GGATTTGGTT
TGGTCTTGTG ATCAGGTGGT CTATGGGGCT ATCCCTACAA AGAAGAATCC AGAAATAGGG
GCACATTGAG GAATGATACT GAGCCCAAAG AGCATTCAAT CATTGTTTTA TTTGCCTTCT
TTTCACACCA TTGGTGAGGG AGGGATTACC ACCCTGGGGT TATGAAGATG GTTGAACACC
CCACACATAG CACCGGAGAT ATGAGATCAA CAGTTTCTTA GCCATAGAGA TTCACAGCCC
AGAGCAGGAG GACGCTGCAC ACCATGCAGG ATGACATGGG GGATGCGCTC GGGATTGGTG
TGAAGAAGCA AGGACTGTTA GAGGCAGGCT TTATAGTAAC AAGACGGTGG GGCAAACTCT
GATTTCCGTG GGGGAATGTC ATGGTCTTGC TTTACTAAGT TTTGAGACTG GCAGGTAGTG
AAACTCATTA GGCTGAGAAC CTTGTGGAAT GCAGCTGACC CAGCTGATAG AGGAAGTAGC
CAGGTGGGAG CCTTTCCCAG TGGGTGTGGG ACATATCTGG CAAGATTTTG TGGCACTCCT
GGTTACAGAT ACTGGGGCAG CAAATAAAAC TGAATCTTGT TTTCAGACCT TAAAAAAAAA
AAAAAAAAAA AA
Microseminoprotein beta (MSMB)
SEQ ID NO: 11
GTACCTGTCT ATAAGGAGTC CTGCTTATCA CAATGAATGT TCTCCTGGGC AGCGTTGTGA
TCTTTGCCAC CTTCGTGACT TTATGCAATG CATCATGCTA TTTCATACCT AATGAGGGAG
TTCCAGGAGA TTCAACCAGG AAATGCATGG ATCTCAAAGG AAACAAACAC CCAATAAACT
CGGAGTGGCA GACTGACAAC TGTGAGACAT GCACTTGCTA CGAAACAGAA ATTTCATGTT
GCACCCTTGT TTCTACACCT GTGGGTTATG ACAAAGACAA CTGCCAAAGA ATCTTCAAGA
AGGAGGACTG CAAGTATATC GTGGTGGAGA AGAAGGACCC AAAAAAGACC TGTTCTGTCA
GTGAATGGAT AATCTAATGT GCTTCTAGTA GGCACAGGGC TCCCAGGCCA GGCCTCATTC
TCCTCTGGCC TCTAATAGTC AATGATTGTG TAGCCATGCC TATCAGTAAA AAGATTTTTG
AGCAAACACT TGAAAAAAAA AAA
Transglutaminase 4 (TGM 4)
SEQ ID NO: 12
GGACCGACTG TGTGGAAGCA CCAGGCATCA GAGATAGAGT CTTCCCTGGC ATTGCAGGAG
AGAATCTGAA GGGATGATGG ATGCATCAAA AGAGCTGCAA GTTCTCCACA TTGACTTCTT
GAATCAGGAC AACGCCGTTT CTCACCACAC ATGGGAGTTC CAAACGAGCA GTCCTGTGTT
CCGGCGAGGA CAGGTGTTTC ACCTGCGGCT GGTGCTGAAC CAGCCCCTAC AATCCTACCA
CCAACTGAAA CTGGAATTCA GCACAGGGCC GAATCCTAGC ATCGCCAAAC ACACCCTGGT
GGTGCTCGAC CCGAGGACGC CCTCAGACCA CTACAACTGG CAGGCAACCC TTCAAAATGA
GTCTGGCAAA GAGGTCACAG TGGCTGTCAC CAGTTCCCCC AATGCCATCC TGGGCAAGTA
CCAACTAAAC GTGAAAACTG GAAACCACAT CCTTAAGTCT GAAGAAAACA TCCTATACCT
TCTCTTCAAC CCATGGTGTA AAGAGGACAT GGTTTTCATG CCTGATGAGG ACGAGCGCAA
AGAGTACATC CTCAATGACA CGGGCTGCCA TTACGTGGGG GCTGCCAGAA GTATCAAATG
CAAACCCTGG AACTTTGGTC AGTTTGAGAA AAATGTCCTG GACTGCTGCA TTTCCCTGCT
GACTGAGAGC TCCCTCAAGC CCACAGATAG GAGGGACCCC GTGCTGGTGT GCAGGGCCAT
GTGTGCTATG ATGAGCTTTG AGAAAGGCCA GGGCGTGCTC ATTGGGAATT GGACTGGGGA
CTACGAAGGT GGCACAGCCC CATACAAGTG GACAGGCAGT GCCCCGATCC TGCAGCAGTA
CTACAACACG AAGCAGGCTG TGTGCTTTGG CCAGTGCTGG GTGTTTGCTG GGATCCTGAC
TACAGTGCTG AGAGCGTTGG GCATCCCAGC ACGCAGTGTG ACAGGCTTCG ATTCAGCTCA
CGACACAGAA AGGAACCTCA CGGTGGACAC CTATGTGAAT GAGAATGGCG AGAAAATCAC
CAGTATGACC CACGACTCTG TCTGGAATTT CCATGTGTGG ACGGATGCCT GGATGAAGCG
ACCGGATCTG CCCAAGGGCT ACGACGGCTG GCAGGCTGTG GACGCAACGC CGCAGGAGCG
AAGCCAGGGT GTCTTCTGCT GTGGGCCATC ACCACTGACC GCCATCCGCA AAGGTGACAT
CTTTATTGTC TATGACACCA GATTCGTCTT CTCAGAAGTG AATGGTGACA GGCTCATCTG
GTTGGTGAAG ATGGTGAATG GGCAGGAGGA GTTACACGTA ATTTCAATGG AGACCACAAG
CATCGGGAAA AACATCAGCA CCAAGGCAGT GGGCCAAGAC AGGCGGAGAG ATATCACCTA
TGAGTACAAG TATCCAGAAG GCTCCTCTGA GGAGAGGCAG GTCATGGATC ATGCCTTCCT
CCTTCTCAGT TCTGAGAGGG AGCACAGACG ACCTGTAAAA GAGAACTTTC TTCACATGTC
GGTACAATCA GATGATGTGC TGCTGGGAAA CTCTGTTAAT TTCACCGTGA TTCTTAAAAG
GAAGACCGCT GCCCTACAGA ATGTCAACAT CTTGGGCTCC TTTGAACTAC AGTTGTACAC
TGGCAAGAAG ATGGCAAAAC TGTGTGACCT CAATAAGACC TCGCAGATCC AAGGTCAAGT
ATCAGAAGTG ACTCTGACCT TGGACTCCAA GACCTACATC AACAGCCTGG CTATATTAGA
TGATGAGCCA GTTATCAGAG GTTTCATCAT TGCGGAAATT GTGGAGTCTA AGGAAATCAT
GGCCTCTGAA GTATTCACGT CTTTCCAGTA CCCTGAGTTC TCTATAGAGT TGCCTAACAC
AGGCAGAATT GGCCAGCTAC TTGTCTGCAA TTGTATCTTC AAGAATACCC TGGCCATCCC
TTTGACTGAC GTCAAGTTCT CTTTGGAAAG CCTGGGCATC TCCTCACTAC AGACCTCTGA
CCATGGGACG GTGCAGCCTG GTGAGACCAT CCAATCCCAA ATAAAATGCA CCCCAATAAA
AACTGGACCC AAGAAATTTA TCGTCAAGTT AAGTTCCAAA CAAGTGAAAG AGATTAATGC
TCAGAAGATT GTTCTCATCA CCAAGTAGCC TTGTCTGATG CTGTGGAGCC TTAGTTGAGA
TTTCAGCATT TCCTACCTTG TGCTTAGCTT TCAGATTATG GATGATTAAA TTTGATGACT
TATATGAGGG CAGATTCAAG AGCCAGCAGG TCAAAAAGGC CAACACAACC ATAAGCAGCC
AGACCCACAA GGCCAGGTCC TGTGCTATCA CAGGGTCACC TCTTTTACAG TTAGAAACAC
CAGCCGAGGC CACAGAATCC CATCCCTTTC CTGAGTCATG GCCTCAAAAA TCAGGGCCAC
CATTGTCTCA ATTCAAATCC ATAGATTTCG AAGCCACAGA GTCTCTCCCT GGAGCAGCAG
ACTATGGGCA GCCCAGTGCT GCCACCTGCT GACGACCCTT GAGAAGCTGC CATATCTTCA
GGCCATGGGT TCACCAGCCC TGAAGGCACC TGTCAACTGG AGTGCTCTCT CAGCACTGGG
ATGGGCCTGA TAGAAGTGCA TTCTCCTCCT ATTGCCTCCA TTCTCCTCTC TCTATCCCTG
AAATCCAGGA AGTCCCTCTC CTGGTGCTCC AAGCAGTTTG AAGCCCAATC TGCAAGGACA
TTTCTCAAGG GCCATGTGGT TTTGCAGACA ACCCTGTCCT CAGGCCTGAA CTCACCATAG
AGACCCATGT CAGCAAACGG TGACCAGCAA ATCCTCTTCC CTTATTCTAA AGCTGCCCCT
TGGGAGACTC CAGGGAGAAG GCATTGCTTC CTCCCTGGTG TGAACTCTTT CTTTGGTATT
CCATCCACTA TCCTGGCAAC TCAAGGCTGC TTCTGTTAAC TGAAGCCTGC TCCTTCTTGT
TCTGCCCTCC AGAGATTTGC TCAAATGATC AATAAGCTTT AAATTAAACT CTACTTCAAA
AAAAAAAAAA AAAAAAAAAA AAAAAAA
Matrix metallopeptidase 10 (stromelysin 2) (MMP10)
SEQ ID NO: 13
AGAAGCCCAG TAGACAAAGA AGGTAAGGGC AGTGAGAATG ATGCATCTTG CATTCCTTGT
GCTGTTGTGT CTGCCAGTCT GCTCTGCCTA TCCTCTGAGT GGGGCAGCAA AAGAGGAGGA
CTCCAACAAG GATCTTGCCC AGCAATACCT AGAAAAGTAC TACAACCTCG AAAAGGATGT
GAAACAGTTT AGAAGAAAGG ACAGTAATCT CATTGTTAAA AAAATCCAAG GAATGCAGAA
GTTCCTTGGG TTGGAGGTGA CAGGGAAGCT AGACACTGAC ACTCTGGAGG TGATGCGCAA
GCCCAGGTGT GGAGTTCCTG ACGTTGGTCA CTTCAGCTCC TTTCCTGGCA TGCCGAAGTG
GAGGAAAACC CACCTTACAT ACAGGATTGT GAATTATACA CCAGATTTGC CAAGAGATGC
TGTTGATTCT GCCATTGAGA AAGCTCTGAA AGTCTGGGAA GAGGTGACTC CACTCACATT
CTCCAGGCTG TATGAAGGAG AGGCTGATAT AATGATCTCT TTTGCAGTTA AAGAACATGG
AGACTTTTAC TCTTTTGATG GCCCAGGACA CAGTTTGGCT CATGCCTACC CACCTGGACC
TGGGCTTTAT GGAGATATTC ACTTTGATGA TGATGAAAAA TGGACAGAAG ATGCATCAGG
CACCAATTTA TTCCTCGTTG CTGCTCATGA ACTTGGCCAC TCCCTGGGGC TCTTTCACTC
AGCCAACACT GAAGCTTTGA TGTACCCACT CTACAACTCA TTCACAGAGC TCGCCCAGTT
CCGCCTTTCG CAAGATGATG TGAATGGCAT TCAGTCTCTC TACGGACCTC CCCCTGCCTC
TACTGAGGAA CCCCTGGTGC CCACAAAATC TGTTCCTTCG GGATCTGAGA TGCCAGCCAA
GTGTGATCCT GCTTTGTCCT TCGATGCCAT CAGCACTCTG AGGGGAGAAT ATCTGTTCTT
TAAAGACAGA TATTTTTGGC GAAGATCCCA CTGGAACCCT GAACCTGAAT TTCATTTGAT
TTCTGCATTT TGGCCCTCTC TTCCATCATA TTTGGATGCT GCATATGAAG TTAACAGCAG
GGACACCGTT TTTATTTTTA AAGGAAATGA GTTCTGGGCC ATCAGAGGAA ATGAGGTACA
AGCAGGTTAT CCAAGAGGCA TCCATACCCT GGGTTTTCCT CCAACCATAA GGAAAATTGA
TGCAGCTGTT TCTGACAAGG AAAAGAAGAA AACATACTTC TTTGCAGCGG ACAAATACTG
GAGATTTGAT GAAAATAGCC AGTCCATGGA GCAAGGCTTC CCTAGACTAA TAGCTGATGA
CTTTCCAGGA GTTGAGCCTA AGGTTGATGC TGTATTACAG GCATTTGGAT TTTTCTACTT
CTTCAGTGGA TCATCACAGT TTGAGTTTGA CCCCAATGCC AGGATGGTGA CACACATATT
AAAGAGTAAC AGCTGGTTAC ATTGCTAGGC GAGATAGGGG GAAGACAGAT ATGGGTGTTT
TTAATAAATC TAATAATTAT TCATCTAATG TATTATGAGC CAAAATGGTT AATTTTTCCT
GCATGTTCTG TGACTGAAGA AGATGAGCCT TGCAGATATC TGCATGTGTC ATGAAGAATG
TTTCTGGAAT TCTTCACTTG CTTTTGAATT GCACTGAACA GAATTAAGAA ATACTCATGT
GCAATAGGTG AGAGAATGTA TTTTCATAGA TGTGTTATTA CTTCCTCAAT AAAAAGTTTT
ATTTTGGGCC TGTTCCTTAA AAAAAAAAAA AAAAAAA
Stanniocalcin 1 (STC1)
SEQ ID NO: 14
CAGTTTGCAA AAGCCAGAGG TGCAAGAAGC AGCGACTGCA GCAGCAGCAG CAGCAGCGGC
GGTGGCAGCA GCAGCAGCAG CGGCGGCAGC AGCAGCAGCA GCGGAGGCAC CGGTGGCAGC
AGCAGCATCA CCAGCAACAA CAACAAAAAA AAATCCTCAT CAAATCCTCA CCTAAGCTTT
CAGTGTATCC AGATCCACAT CTTCACTCAA GCCAGGAGAG GGAAAGAGGA AAGGGGGGCA
GGAAAAAAAA AAAACCCAAC AACTTAGCGG AAACTTCTCA GAGAATGCTC CAAAACTCAG
CAGTGCTTCT GGTGCTGGTG ATCAGTGCTT CTGCAACCCA TGAGGCGGAG CAGAATGACT
CTGTGAGCCC CAGGAAATCC CGAGTGGCGG CTCAAAACTC AGCTGAAGTG GTTCGTTGCC
TCAACAGTGC TCTACAGGTC GGCTGCGGGG CTTTTGCATG CCTGGAAAAC TCCACCTGTG
ACACAGATGG GATGTATGAC ATCTGTAAAT CCTTCTTGTA CAGCGCTGCT AAATTTGACA
CTCAGGGAAA AGCATTCGTC AAAGAGAGCT TAAAATGCAT CGCCAACGGG GTCACCTCCA
AGGTCTTCCT CGCCATTCGG AGGTGCTCCA CTTTCCAAAG GATGATTGCT GAGGTGCAGG
AAGAGTGCTA CAGCAAGCTG AATGTGTGCA GCATCGCCAA GCGGAACCCT GAAGCCATCA
CTGAGGTCGT CCAGCTGCCC AATCACTTCT CCAACAGATA CTATAACAGA CTTGTCCGAA
GCCTGCTGGA ATGTGATGAA GACACAGTCA GCACAATCAG AGACAGCCTG ATGGAGAAAA
TTGGGCCTAA CATGGCCAGC CTCTTCCACA TCCTGCAGAC AGACCACTGT GCCCAAACAC
ACCCACGAGC TGACTTCAAC AGGAGACGCA CCAATGAGCC GCAGAAGCTG AAAGTCCTCC
TCAGGAACCT CCGAGGTGAG GAGGACTCTC CCTCCCACAT CAAACGCACA TCCCATGAGA
GTGCATAACC AGGGAGAGGT TATTCACAAC CTCACCAAAC TAGTATCATT TTAGGGGTGT
TGACACACCA GTTTTGAGTG TACTGTGCCT GGTTTGATTT TTTTAAAGTA GTTCCTATTT
TCTATCCCCC TTAAAGAAAA TTGCATGAAA CTAGGCTTCT GTAATCAATA TCCCAACATT
CTGCAATGGC AGCATTCCCA CCAACAAAAT CCATGTGACC ATTCTGCCTC TCCTCAGGAG
AAAGTACCCT CTTTTACCAA CTTCCTCTGC CATGTTTTTC CCCTGCTCCC CTGAGACCAC
CCCCAAACAC AAAACATTCA TGTAACTCTC CAGCCATTGT AATTTGAAGA TGTGGATCCC
TTTAGAACGG TTGCCCCAGT AGAGTTAGCT GATAAGGAAA CTTTATTTAA ATGCATGTCT
TAAATGCTCA TAAAGATGTT AAATGGAATT CGTGTTATGA ATCTGTGCTG GCCATGGACG
AATATGAATG TCACATTTGA ATTCTTGATC TCTAATGAGC TAGTGTCTTA TGGTCTTGAT
CCTCCAATGT CTAATTTTCT TTCCGACACA TTTACCAAAT TGCTTGAGCC TGGCTGTCCA
ACCAGACTTT GAGCCTGCAT CTTCTTGCAT CTAATGAAAA ACAAAAAGCT AACATCTTTA
CGTACTGTAA CTGCTCAGAG CTTTAAAAGT ATCTTTAACA ATTGTCTTAA AACCAGAGAA
TCTTAAGGTC TAACTGTGGA ATATAAATAG CTGAAAACTA ATGTACTGTA CATAAATTCC
AGAGGACTCT GCTTAAACAA AGCAGTATAT AATAACTTTA TTGCATATAG ATTTAGTTTT
GTAACTTAGC TTTATTTTTC TTTTCCTGGG AATGGAATAA CTATCTCACT TCCAGATATC
CACATAAATG CTCCTTGTGG CCTTTTTTAT AACTAAGGGG GTAGAAGTAG TTTTAATTCA
ACATCAAAAC TTAAGATGGG CCTGTATGAG ACAGGAAAAA CCAACAGGTT TATCTGAAGG
ACCCCAGGTA AGATGTTAAT CTCCCAGCCC ACCTCAACCC AGAGGCTACT CTTGACTTAG
ACCTATACTG AAAGATCTCT GTCACATCCA ACTGGAAATT CCAGGAACCA AAAAGAGCAT
CCCTATGGGC TTGGACCACT TACAGTGTGA TAAGGCCTAC TATACATTAG GAAGTGGCAG
TTCTTTACTC GTCCCCTTTC ATCGGTGCCT GGTACTCTGG CAAATGATGA TGGGGTGGGA
GACTTTCCAT TAAATCAATC AGGAATGAGT CAATCAGCCT TTAGGTCTTT AGTCCGGGGG
ACTTGGGGCT GAGAGAGTAT AAATAACCCT GGGCTGTCCA GCCTTAATAG ACTTCTCTTA
CATTTTCGTC CTGTAGCACG CTGCCTGCCA AAGTAGTCCT GGCAGCTGGA CCATCTCTGT
AGGATCGTAA AAAAATAGAA AAAAAGAAAA AAAAAAGAAA GAAAGAGGGA AAAAGAGCTG
GTGGTTTGAT CATTTCTGCC ATGATGTTTA CAAGATGGCG ACCACCAAAG TCAAACGACT
AACCTATCTA TGAACAACAG TAGTTTCTCA GGGTCACTGT CCTTGAACCC AACAGTCCCT
TATGAGCGTC ACTGCCCACC AAAGGTCAAT GTCAAGAGAG GAAGAGAGGG AGGAGGGGTA
GGACTGCAGG GGCCACTCCA AACTCGCTTA GGTAGAAACT ATTGGTGCTT GACTCTCACT
AGGCTAAACT CAAGATTTGA CCAAATCGAG TGATAGGGAT CCTGGTGGGA GGAGAGAGGG
CACATCTCCA GAAAAATGAA AAGCAATACA ACTTTACCAT AAAGCCTTTA AAACCAGTAA
CGTGCTGCTC AAGGACCAAG AGCAATTGCA GCAGACCCAG CAGCAGCAGC AGCAGCACAA
ACATTGCTGC CTTTGTCCCC ACACAGCCTC TAAGCGTGCT GACATCAGAT TGTTAAGGGC
ATTTTTATAC TCAGAACTGT CCCATCCCCA GGTCCCCAAA CTTATGGACA CTGCCTTAGC
CTCTTGGAAA TCAGGTAGAC CATATTCTAA GTTAGACTCT TCCCCTCCCT CCCACACTTC
CCACCCCCAG GCAAGGCTGA CTTCTCTGAA TCAGAAAAGC TATTAAAGTT TGTGTGTTGT
GTCCATTTTG CAAACCCAAC TAAGCCAGGA CCCCAATGCG ACAAGTAGTT CATGAGTATT
CCTAGCAAAT TTCTCTCTTT CTTCAGTTCA GTAGATTTCC TTTTTTCTTT TCTTTTTTTT
TTTTTTTTTT TTTGGCTGTG ACCTCTTCAA ACCGTGGTAC CCCCCCTTTT CTCCCCACGA
TGATATCTAT ATATGTATCT ACAATACATA TATCTACACA TACAGAAAGA AGCAGTTCTC
ACAATGTTGC TAGTTTTTTG CTTCTCTTTC CCCCACCCTA CTCCCTCCAA TTCCCCCTTA
AACTTCCAAA GCTTCGTCTT GTGTTTGCTG CAGAGTGATT CGGGGGCTGA CCTAGACCAG
TTTGCATGAT TCTTCTCTTG TGATTTGGTT GCACTTTAGA CATTTTTGTG CCATTATATT
TGCATTATGT ATTTATAATT TAAATGATAT TTAGGTTTTT GGCTGAGTAC TGGAATAAAC
AGTGAGCATA TCTGGTATAT GTCATTATTT ATTGTTAAAT TACATTTTTA AGCTCCATGT
GCATATAAAG GTTATGAAAC ATATCATGGT AATGACAGAT GCAAGTTATT TTATTTGCTT
ATTTTTATAA TTAAAGATGC CATAGCATAA TATGAAGCCT TTGGTGAATT CCTTCTAAGA
TAAAAATAAT AATAAAGTGT TACGTTTTAT TGGTTTCAAA AAAAAAAAAA AAAAAAA
Matrix metallopeptidase 3 (MMP3)
SEQ ID NO: 15
AAAGCAAGGA TGAGTCAAGC TGCGGGTGAT CCAAACAAAC ACTGTCACTC TTTAAAAGCT
GCGCTCCCGA GGTTGGACCT ACAAGGAGGC AGGCAAGACA GCAAGGCATA GAGACAACAT
AGAGCTAAGT AAAGCCAGTG GAAATGAAGA GTCTTCCAAT CCTACTGTTG CTGTGCGTGG
CAGTTTGCTC AGCCTATCCA TTGGATGGAG CTGCAAGGGG TGAGGACACC AGCATGAACC
TTGTTCAGAA ATATCTAGAA AACTACTACG ACCTCAAAAA AGATGTGAAA CAGTTTGTTA
GGAGAAAGGA CAGTGGTCCT GTTGTTAAAA AAATCCGAGA AATGCAGAAG TTCCTTGGAT
TGGAGGTGAC GGGGAAGCTG GACTCCGACA CTCTGGAGGT GATGCGCAAG CCCAGGTGTG
GAGTTCCTGA TGTTGGTCAC TTCAGAACCT TTCCTGGCAT CCCGAAGTGG AGGAAAACCC
ACCTTACATA CAGGATTGTG AATTATACAC CAGATTTGCC AAAAGATGCT GTTGATTCTG
CTGTTGAGAA AGCTCTGAAA GTCTGGGAAG AGGTGACTCC ACTCACATTC TCCAGGCTGT
ATGAAGGAGA GGCTGATATA ATGATCTCTT TTGCAGTTAG AGAACATGGA GACTTTTACC
CTTTTGATGG ACCTGGAAAT GTTTTGGCCC ATGCCTATGC CCCTGGGCCA GGGATTAATG
GAGATGCCCA CTTTGATGAT GATGAACAAT GGACAAAGGA TACAACAGGG ACCAATTTAT
TTCTCGTTGC TGCTCATGAA ATTGGCCACT CCCTGGGTCT CTTTCACTCA GCCAACACTG
AAGCTTTGAT GTACCCACTC TATCACTCAC TCACAGACCT GACTCGGTTC CGCCTGTCTC
AAGATGATAT AAATGGCATT CAGTCCCTCT ATGGACCTCC CCCTGACTCC CCTGAGACCC
CCCTGGTACC CACGGAACCT GTCCCTCCAG AACCTGGGAC GCCAGCCAAC TGTGATCCTG
CTTTGTCCTT TGATGCTGTC AGCACTCTGA GGGGAGAAAT CCTGATCTTT AAAGACAGGC
ACTTTTGGCG CAAATCCCTC AGGAAGCTTG AACCTGAATT GCATTTGATC TCTTCATTTT
GGCCATCTCT TCCTTCAGGC GTGGATGCCG CATATGAAGT TACTAGCAAG GACCTCGTTT
TCATTTTTAA AGGAAATCAA TTCTGGGCTA TCAGAGGAAA TGAGGTACGA GCTGGATACC
CAAGAGGCAT CCACACCCTA GGTTTCCCTC CAACCGTGAG GAAAATCGAT GCAGCCATTT
CTGATAAGGA AAAGAACAAA ACATATTTCT TTGTAGAGGA CAAATACTGG AGATTTGATG
AGAAGAGAAA TTCCATGGAG CCAGGCTTTC CCAAGCAAAT AGCTGAAGAC TTTCCAGGGA
TTGACTCAAA GATTGATGCT GTTTTTGAAG AATTTGGGTT CTTTTATTTC TTTACTGGAT
CTTCACAGTT GGAGTTTGAC CCAAATGCAA AGAAAGTGAC ACACACTTTG AAGAGTAACA
GCTGGCTTAA TTGTTGAAAG AGATATGTAG AAGGCACAAT ATGGGCACTT TAAATGAAGC
TAATAATTCT TCACCTAAGT CTCTGTGAAT TGAAATGTTC GTTTTCTCCT GCCTGTGCTG
TGACTCGAGT CACACTCAAG GGAACTTGAG CGTGAATCTG TATCTTGCCG GTCATTTTTA
TGTTATTACA GGGCATTCAA ATGGGCTGCT GCTTAGCTTG CACCTTGTCA CATAGAGTGA
TCTTTCCCAA GAGAAGGGGA AGCACTCGTG TGCAACAGAC AAGTGACTGT ATCTGTGTAG
ACTATTTGCT TATTTAATAA AGACGATTTG TCAGTTATTT TATCTT
(polynucleotide, matrix metallopeptidase 11 (MMP11)
SEQ ID NO: 16
AAGCCCAGCA GCCCCGGGGC GGATGGCTCC GGCCGCCTGG CTCCGCAGCG CGGCCGCGCG
CGCCCTCCTG CCCCCGATGC TGCTGCTGCT GCTCCAGCCG CCGCCGCTGC TGGCCCGGGC
TCTGCCGCCG GACGCCCACC ACCTCCATGC CGAGAGGAGG GGGCCACAGC CCTGGCATGC
AGCCCTGCCC AGTAGCCCGG CACCTGCCCC TGCCACGCAG GAAGCCCCCC GGCCTGCCAG
CAGCCTCAGG CCTCCCCGCT GTGGCGTGCC CGACCCATCT GATGGGCTGA GTGCCCGCAA
CCGACAGAAG AGGTTCGTGC TTTCTGGCGG GCGCTGGGAG AAGACGGACC TCACCTACAG
GATCCTTCGG TTCCCATGGC AGTTGGTGCA GGAGCAGGTG CGGCAGACGA TGGCAGAGGC
CCTAAAGGTA TGGAGCGATG TGACGCCACT CACCTTTACT GAGGTGCACG AGGGCCGTGC
TGACATCATG ATCGACTTCG CCAGGTACTG GCATGGGGAC GACCTGCCGT TTGATGGGCC
TGGGGGCATC CTGGCCCATG CCTTCTTCCC CAAGACTCAC CGAGAAGGGG ATGTCCACTT
CGACTATGAT GAGACCTGGA CTATCGGGGA TGACCAGGGC ACAGACCTGC TGCAGGTGGC
AGCCCATGAA TTTGGCCACG TGCTGGGGCT GCAGCACACA ACAGCAGCCA AGGCCCTGAT
GTCCGCCTTC TACACCTTTC GCTACCCACT GAGTCTCAGC CCAGATGACT GCAGGGGCGT
TCAACACCTA TATGGCCAGC CCTGGCCCAC TGTCACCTCC AGGACCCCAG CCCTGGGCCC
CCAGGCTGGG ATAGACACCA ATGAGATTGC ACCGCTGGAG CCAGACGCCC CGCCAGATGC
CTGTGAGGCC TCCTTTGACG CGGTCTCCAC CATCCGAGGC GAGCTCTTTT TCTTCAAAGC
GGGCTTTGTG TGGCGCCTCC GTGGGGGCCA GCTGCAGCCC GGCTACCCAG CATTGGCCTC
TCGCCACTGG CAGGGACTGC CCAGCCCTGT GGACGCTGCC TTCGAGGATG CCCAGGGCCA
CATTTGGTTC TTCCAAGGTG CTCAGTACTG GGTGTACGAC GGTGAAAAGC CAGTCCTGGG
CCCCGCACCC CTCACCGAGC TGGGCCTGGT GAGGTTCCCG GTCCATGCTG CCTTGGTCTG
GGGTCCCGAG AAGAACAAGA TCTACTTCTT CCGAGGCAGG GACTACTGGC GTTTCCACCC
CAGCACCCGG CGTGTAGACA GTCCCGTGCC CCGCAGGGCC ACTGACTGGA GAGGGGTGCC
CTCTGAGATC GACGCTGCCT TCCAGGATGC TGATGGCTAT GCCTACTTCC TGCGCGGCCG
CCTCTACTGG AAGTTTGACC CTGTGAAGGT GAAGGCTCTG GAAGGCTTCC CCCGTCTCGT
GGGTCCTGAC TTCTTTGGCT GTGCCGAGCC TGCCAACACT TTCCTCTGAC CATGGCTTGG
ATGCCCTCAG GGGTGCTGAC CCCTGCCAGG CCACGAATAT CAGGCTAGAG ACCCATGGCC
ATCTTTGTGG CTGTGGGCAC CAGGCATGGG ACTGAGCCCA TGTCTCCTCA GGGGGATGGG
GTGGGGTACA ACCACCATGA CAACTGCCGG GAGGGCCACG CAGGTCGTGG TCACCTGCCA
GCGACTGTCT CAGACTGGGC AGGGAGGCTT TGGCATGACT TAAGAGGAAG GGCAGTCTTG
GGCCCGCTAT GCAGGTCCTG GCAAACCTGG CTGCCCTGTC TCCATCCCTG TCCCTCAGGG
TAGCACCATG GCAGGACTGG GGGAACTGGA GTGTCCTTGC TGTATCCCTG TTGTGAGGTT
CCTTCCAGGG GCTGGCACTG AAGCAAGGGT GCTGGGGCCC CATGGCCTTC AGCCCTGGCT
GAGCAACTGG GCTGTAGGGC AGGGCCACTT CCTGAGGTCA GGTCTTGGTA GGTGCCTGCA
TCTGTCTGCC TTCTGGCTGA CAATCCTGGA AATCTGTTCT CCAGAATCCA GGCCAAAAAG
TTCACAGTCA AATGGGGAGG GGTATTCTTC ATGCAGGAGA CCCCAGGCCC TGGAGGCTGC
AACATACCTC AATCCTGTCC CAGGCCGGAT CCTCCTGAAG CCCTTTTCGC AGCACTGCTA
TCCTCCAAAG CCATTGTAAA TGTGTGTACA GTGTGTATAA ACCTTCTTCT TCTTTTTTTT
TTTTTAAACT GAGGATTGTC ATTAAACACA GTTGTTTTCT AAAAAAAAAA AAAAAA
Cytochrome P450 family 2 subfamily B member 7 pseudogene
(CYP2B7P1)
SEQ ID NO: 17
CTGGAACCAT GGAGCTCAGC GTCCTCCTCT TCCTTGCACT CCTCACAGGC CTCTTGCTAC
TCCTGGTTCA GCGTCACCCT AACTCCCATG GCACCCTCCC ACCAGGGCCC CGCCCTCTGC
CCCTTTTGGG GAACCTTCTG CAGATGGACA GAAGAGGCCT ACTCAAATCC TTTCTGAGGT
TCCGAGAGAA ATATGGGGAC GTCTTCACGG TACACCTGGG ACCGAGGCCC GTGGTCATGC
TGTGTGGAGT AGAGGCCATA CGGGAGGCCC TGGTGGACAA CGCTGAGGCC TTCTCTGGCC
GGGGAAAAAT CGTCATCATG GACCCAGTCT ACCAGGGATA TGGCATGCTC TTTGCCAATG
GAAACCGCTG GAAGGTGCTT CGGCGATTCT CTGTGACCAC CATGAGGGAC TTCGGGATGG
GAAAGCGGAG TGTGGAGGAG CGGATTCAGG ACGAGGCTCA GTGTCTGATA GAGGAACTTC
GGAAATCCAA GGGAGCCCTC GTGGACCCCA CCTTCCTCTT CCATTCCATT ACCGCCAACA
TCATCTGCTC CATCATCTTT GGAAAACGCT TCCACTACCA AGATCAAGAG TTCCTGAAGA
CGCTGAACTT GTTCTGCCAG AGTTTCTTAC TCATCAGCTC TATATCCAGC CAGCTGTTTG
AGCTCTTCTC TGGCTTCTTG AAATACTTTC CTGGGGCACA CAGGCAAGTT TACAAAAACC
TACAGGAAAT CAATGCTTAC ATTGGCCACA GTGTGGAGAA GCACCGTGAA ACCCTGGACC
CCAGCGCCCC CAGGGACCTC ATCGACACCT ACCTGCTCCA CATGGAAAAA GAGAAATCCA
ACCCACACAG TGAATTCAGC CACCAGAACC TCATCATCAA CACGCTCTCG CTCTTCTTTG
CTGGCACTGA GACCACCAGC ACCACTCTCC GCTACGGCTT CCTGCTCATG CTCAAATACC
CTCATGTCGC AGAGAGAGTC TACAAGGAGA TTGAACAGGT GGTTGGCCCA CATCGCCCTC
CAGCGCTTGA TGACCGAGCC AAAATGCCAT ACACAGAGGC AGTCATCCGT GAGATTCAGA
GATTTGCTGA CCTTCTCCCC ATGGGTGTGC CCCACATTGT CACCCAACAC ACCAGCTTCT
GAGGGTACAC CATCCCCAAG GACACGGAAG TATTTCTCAT CCTGAGCACT GCTCTCCGTG
ACCCACACTA CTTTGAAAAA CCAGACGCCT TCAATCCTGA CCACTTTCTG GATGCCAATG
GGGCACTGAA AAAGAATGAA GCTTTTATCC CCTTCTCCTT AGGGAAGCGG ATTTGTCTTG
GTGAAGGCAT TGCCCGTGCG GAATTGTTCC TCTTCTTCAC CACCATCCTC CAGAACTTCT
CCGTGGCCAG CCCCGTGGCT CCTGAAGACA TCGATCTGAC ACCCCAGGAG TGTGGTGTGG
GCAAAATACC CCCAACATAC CAGATCTGCT TCCTGCCCCG CTGAAGGGGC TGAGGGAAGG
GGGTCAAAGG ATTCCAGGGT CATTCAGTGT CCCCACCTCT GTAGATAATG GCTCTGACTC
CCTGCAACTT CCTGCCTCTG AGAGACCTGC TGCAAGCCAG CTTCCTTCCC TTCCATGGCA
CCAGTTGTCT GAGGTCGCAG TGCAAATGAG TGGAGGAGTG AGATTATTGA AAATTATAAT
ATACAAAATT ATATATATAT ATTTTGAGAC AGAGTCTCAC TCAGTTGCCC AGGCTGGAGT
GCAGTGGCGT GATCTCGGCT CACTGCAACC TCCACCCCCG GGGTTCAAGA AATTCTCCTG
CCTCAGCCTC CCTAGTAGCT GGGATTACAG GTGTGTGCTA CCATGCCTGG CTAATTTTTG
TATTTTTAGT AGAGATGGGG TTTCACCGTG TTGGCCAGGC TGATCTCAAA CTCCTGAACT
CAAGTGATTC ACCCACCTTA GCCTCCCAAA GTGCTGGGAT TACAGGTGTG AGTCACCATG
CCCGGCCATG TATATATATA ATTTTAAAAA TTAAGATGAA ATTCACATAA AATAAAATTA
GCCATTTTAA AGTGTACAAT TTAGTGGTGT GTGGTTCATT CACAAAGCTG TACAACCACC
ACCATCTAGT TCCAAACATT TTCTTTTTTT CTGAGACGGA GTCTCACTCT GTCACCCAGG
TTCGAGTTCA GTGGTCTTGA ACTCCTGATG TCAGGTGATT CTCCTAGTTC CAAATGTTTT
CATTATCTCC CCCCAACAAA ACCCATACCT ATCAAGCTGT CACTCCCCAT ACCCCATTCT
CTTTTTCATC TCAGCCCCTG TCAATCTGGT TTTTGTCCTT ATGGACTTAC CAATTCTGAA
TATTTCCTAT AAACAGAATC ACACAATATT TGATTTTTTT TTTAAAACTA AGCCTTGCTC
TGTCTCCCAG GCTGGAGTGC TGTGGCGTGA TTTTGGTTCA CTGCAACCTC CGCCTTCCAA
GTTCAAGAGA TTCTCCTGCC TCAGCTTCCA AGTAGCTGGG ATTACAGGCA TGTGGTACCA
CGCCTGGCTA ATTTTCTTGT ATTTTTAGTA GGGACATGTT GGCCAGGCTG GTTGTGAGCT
CCTGGCCTCA GGTGATCCAC ACGCCTCAGT GTCCCAGAGT GCTGATATTA CAGGCGTAAT
ATGTGATCTT TTGTGTCTGG TTCCTTTCAC GTTGAACGCT ATTTTTGAGG TTCGTGCCTG
TTGTAGACCA CAGTCACACA CTGCTGTAGT CTTCCCCCAT CCTCATTCCC AGCTGCCTCC
TCCTACTGTT TCCCTCTATC AAAAAGCCTC CTTGGCGCAG GTTCCCTGAG CTGTGGGATT
CTGCACTGGT GCTTTGGATT CCCTGATATG TTCCTTCAAA TCCACTGAGA ATTAAATAAA
CATCGCTAAA GCATGACCTC CCCACGTCAA AAAAAAAAAA AAAAAAAAAA AAAAAAAAAA
AAAAAAAAAA AAAAAAAAAA AAAAAAAAAA AAAAAAAAAA AAAAAAAAAA AAAAAAAAAA
Lactobacillus gasseri
SEQ ID NO: 18
CAATGGACGC AAGTCTGATG GAGCAACGCC GCGTGAGTGA AGAAGGGTTT CGACTCGTAA
AGCTCTGTTG GTAGTGAAGA AAGATAGAGG TAGTAACTGG CCTTTATTTG ACGGTAATTA
CTTAGAAAGT CACGGCTAAC TACGTGCCAG CAGCCGCGGT AATACGTAGG TGGCAAGCGT
TGTCCGGATT TATTGGGCGT AAAGCGAGTG CAGGCGGTTC AATAAGTCTG ATGTGAAAGC
CTTCGGCTCA ACCGGAGAAT TGCATCAGAA ACTGTTGAAC TTGAGTGCAG AAGAGGAGAG
TGGAACTCCA TGTGTAGCGG TGGAATGCGT AGATATATGG AAGAACACCA GTGGCGAAGG
CGGCTCTCTG GTCTGCAACT GACGCTGAGG CTCGAAAGCA TGGGTAGCGA ACAGGATTAG
ATACCCTGGT AGTCCATGCC GTAAACGATG AGTGCTAAGT GTTGGGAGGT TTCCGCCTCT
CAGTGCTGCA GCTAACGCAT TAAGCACTCC GCCTGGGGAG TACGACCGCA AGGTTGAAAC
TCAAAGGAAT TGACGGGGGC CCGCACAAGC GGTGGAGCAT GTGGTTTAAT TCGAAGCAAC
GCGAAGAACC TTACCAGGTC TTGACATCCA GTGCAAGCCT AAGAGATTAG GAGTTCCCTT
CGGGGACGCT GAGACAGGTG GTGCATGGCT GTCGTCAGCT CGTGTCGTGA GATGTTGGGT
TAAGTCCCGC AACGAGCGCA ACCCTTGTCA TTAGTTGCCA TCATTAAGTT GGGCACTCTA
ATGAGACTGC CGGTGACAAA CCGGAGGAAG GTGGGGATGA CGTCAAGTCA TCATGCCCCT
TATGACCTGG GCTACACACG TGCTACAATG GACGGTACAA CGAGAAGCGA ACCTTCGAAG
GCAAGCGGAT CTCTGAAAGC CGTTCTCAGT TCGGACTGTA GGCTGCAACT CGCCTACACG
AAGCTGGAAT CGCTAGTAAT CGCGGATCAG CACGCCGCGG TGAATACGTT CCCGGG
Lactobacillus crispatus
SEQ ID NO: 19
CGGCGTGCCT AATACATGCA AGTCGAGCGA GCGGAACTAA CAGATTTACT TCGGTAATGA
CGTTAGGAAA GCGAGCGGCG GATGGGTGAG TAACACGTGG GGAACCTGCC CCATAGTCTG
GGATACCACT TGGAAACAGG TGCTAATACC GGATAAGAAA GCAGATCGCA TGATCAGCTT
TTNAAAGGCG GCGTAAGCTG TCGCTATGGG ATGGCCCCGC GGTGCATTAG CTAGTTGGTA
AGGTAAAGGC TTACCAAGGC GATGATGCAT AGCCGAGTTG AGAGACTGAT CGGCCACATT
GGGACTGAGA CACGGCCCAA ACTCCTACGG GAGGCAGCAG TAGGGAATCT TCCACAATGG
ACGCAAGTCT GATGGAGCAA CGCCGCGTGA GTGAAGAAGG TTTTCGGATC GTAAAGCTCT
GTTGTTGGTG AAGAAGGATA GAGGTAGTAA CTGGCCTTTA TTTGACGGTA ATCAACCAGA
AAGTCACGGC TAACTACGTG CCAGCAGCCG CGGTAATACG TAGGTGGCAA GCGTTGTCCG
GATTTATTGG GCGTAAAGCG AGCGCAGGCG GAAGAATAAG TCTGATGTGA AAGCCCTCGG
CTTAACCGAG GAACTGCATC GGAAACTGTT TTTCTTGAGT GCAGAAGAGG AGAGTGGAAC
TCCATGTGTA GCGGTGGAAT GCGTAGATAT ATGGAAGAAC ACCAGTGGCG AAGGCGGCTC
TCTGGTCTGC AACTGACGCT GAGGCTCGAA AGCATGGGTA GCGAACAGGA TTAGATACCC
TGGTAGTCCA TGCCGTAAAC GATGAGTGCT AAGTGTTGGG AGGTTTCCGC CTCTCAGTGC
TGCAGCTAAC GCATTAAGCA CTCCGCCTGG GGAGTACGAC CGCAAGGTTG AAACTCAAAG
GAATTGACGG GGGCCCGCAC AAGCGGTGGA GCATGTGGTT TAATTCGAAG CAACGCGAAG
AACCTTACCA GGTCTTGACA TCTAGTGCCA TTTGTAGAGA TACAAAGTTC CCTTCGGGGA
CGCTAAGACA GGTGGTGCAT GGCTGTCGTC AGCTCGTGTC GTGAGATGTT GGGTTAAGTC
CCGCAACGAG CGCAACCCTT GTTATTAGTT GCCAGCATTA AGTTGGGCAC TCTAATGAGA
CTGCCGGTGA CAAACCGGAG GAAGGTGGGG ATGACGTCAA GTCATCATGC CCCTTATGAC
CTGGGCTACA CACGTGCTAC AATGGGCAGT ACAACGAGAA GCGAGCCTGC GAAGGCAAGC
GAATCTCTGA AAGCTGTTCT CAGTTCGGAC TGCAGTCTGC AACTCGACTG CACGAAGCTG
Hemoglobin delta (HBD)
SEQ ID NO: 20
ACTGCTGTCA ATGCCCTGTG
Hemoglobin delta (HBD)
SEQ ID NO: 21
ACCTTCTTGC CATGAGCCTT
Solute carrier family 4 (anion exchanger), member 1 (Diego blood group)
(SLC4A1)
SEQ ID NO: 22
AACTGGACAC TCAGGACCAC
Solute carrier family 4 (anion exchanger), member 1 (Diego blood group)
(SLC4A1)
SEQ ID NO: 23
GGATGTCTGG GTCTTCATAT TCCT
Glycophorin A (MNS blood group) (GYPA)
SEQ ID NO: 24
CAGACAAATG ATACGCACAA ACG
Glycophorin A (MNS blood group) (GYPA)
SEQ ID NO: 25
CCAATAACAC CAGCCATCAC C
Follicular dendritic cell secreted protein (FDCSP)
SEQ ID NO: 26
CTCTCAAGAC CAGGAACGAG AA
Follicular dendritic cell secreted protein (FDCSP)
SEQ ID NO: 27
GGGCAGATTC AGGTATTGGA ATAG
Histatin 3 (HTN3)
SEQ ID NO: 28
AAGCATCATT CACATCGAGG CTAT
Histatin 3 (HTN3)
SEQ ID NO: 29
ATGCGGTATG ACAAATGAGA ATACAC
Statherin
SEQ ID NO: 30
CTTGAGTAAA AGAGAACCC AGCCA
Statherin
SEQ ID NO: 31
TTCTGGAACT GGCTGATAAG GG
Protamine 1 (PRM1)
SEQ ID NO: 32
GCCAGGTACA GATGCTGTCG CAG
Protamine 1 (PRM1)
SEQ ID NO: 33
GTGTCTTCTA CATCTCGGTC TG
Transition protein 1 (TNP1)
SEQ ID NO: 34
GATGACGCCA ATCGCAATTA CC
Transition protein 1 (TNP1)
SEQ ID NO: 35
CCTTCTGCTG TTCTTGTTGC TG
Protamine 2 (PRM2)
SEQ ID NO: 36
CGTGAGGAGC CTGAGCGA
Protamine 2 (PRM2)
SEQ ID NO: 37
CGATGCTGCC GCCTGT
Kallikrein related peptidase 2 (KLK2)
SEQ ID NO: 38
TTCTCTCCAT CGCCTTGTCT G
Kallikrein related peptidase 2 (KLK2)
SEQ ID NO: 39
AGTGTGCCCA TCCATGACTG
Microsemino protein beta (MSMB)
SEQ ID NO: 40
CTTTGCCACC TTCGTGACTT TATG
Microsemino protein beta (MSMB)
SEQ ID NO: 41
ACAGTTGTCA GTCTGCCACT
Transglutaminase 4 (TGM 4)
SEQ ID NO: 42
TGAGAAAGGC CAGGGCG
Transglutaminase 4 (TGM 4)
SEQ ID NO: 43
AATCGAAGCC TGTCACACTG C
Matrix metallopeptidase 10 (stromelysin 2) (MMP10)
SEQ ID NO: 44
CCCACTCTAC AACTCATTCA CAGAG
Matrix metallopeptidase 10 (stromelysin 2) (MMP10)
SEQ ID NO: 45
GGTTCCTCAG TAGAGGCAGG
Stanniocalcin 1 (STC1)
SEQ ID NO: 46
CTGCCCAATC ACTTCTCCAA CA
Stanniocalcin 1 (STC1)
SEQ ID NO: 47
TTTCTCCATC AGGCTGTCTC T
Matrix metallopeptidase 3 (MMP3)
SEQ ID NO: 48
CCATGCCTAT GCCCCTG
Matrix metallopeptidase 3 (MMP3)
SEQ ID NO: 49
GTCCCTGTTG TATCCTTTGT CC
(Matrix metallopeptidase 11 (MMP11)
SEQ ID NO: 50
CAAGACTCAC CGAGAAGGGG
(Matrix metallopeptidase 11 (MMP11)
SEQ ID NO: 51
GCCTTGGCTG CTGTTGTGT
Cytochrome P450 family 2 subfamily B member 7 pseudogene
(CYP2B7P1)
CCGTGAGATT CAGAGATTTG CTGAC
Cytochrome P450 family 2 subfamily B member 7 pseudogene
(CYP2B7P1)
SEQ ID NO: 53
TGAGAAATAC TTCCGTGTCC TTGG
Lactobacillus gasseri
SEQ ID NO: 54
CAGAGCAAGC GGAAGCACA
Lactobacillus gasseri/Lactobacillus crispatus
SEQ ID NO: 55
TTGCTTACTT ACTGCTCCCC G
Lactobacillus crispatus
SEQ ID NO: 56
GAGAAAGCCA AGCGGAAGC
Lactobacillus gasseri/Lactobacillus crispatus
SEQ ID NO: 57
TTGCTTACTT ACTGCTCCCC G

1. A method for determining the type of a biological sample, comprising the steps of
detecting RNA from the sample associated with any one or more of HBD, SLC4A1, GYPA, FDCSP, HTN3, STATH, PRM1, TNP1, PRM2, KLK2, MSMB, TGM4, MMP10, STC1, MMP3, MMP11, CYP2B7P, Lactobacillus gasseri (L.gass) and Lactobacillus crispatus (L.crisp) and
determining whether the sample is circulatory blood, saliva, spermatozoa, seminal fluid, menstrual fluid or vaginal material.

2. The method of claim 1, comprising detecting an RNA associated with one or more of SEQ ID Nos: 1 to 19.

3. The method of claim 1, wherein the step of detecting the RNA includes the use of one or more primers specific for any one or more of HBD, SLC4A1, GYPA, FDCSP, HTN3, STATH, PRM1, TNP1, PRM2, KLK2, MSMB, TGM4, MMP10, STC1, MMP3, MMP11, CYP2B7P, Lactobacillus gasseri (L.gass) and Lactobacillus crispatus (L.crisp).

4. The method of claim 3, wherein the one or more primers are selected from SEQ ID Nos: 20 to 57.

5. The method of claim 1, further comprising determining if the biological sample is circulatory blood, comprising the step of detecting RNA associated with HBD using primers of SEQ ID No: 20 and 21, and/or SLC4A1 using primers of SEQ ID No:22 and 23 and/or GYPA using primers of SEQ ID No: 24 and 25.

6. The method of claim 1, further comprising determining if the biological sample is saliva, comprising the step of detecting RNA associated with FDCSP using primers of SEQ ID No: 26 and 27, and/or HTN3 using primers of SEQ ID No: 28 and 29, and/or STATH using primers of SEQ ID No: 30 and 31.

7. The method of claim 1, further comprising determining if the biological sample is spermatozoa, comprising the step of detecting RNA associated with PRM1 using primers of SEQ ID No:32 and 33 and/or TNP1 using primers of SEQ ID No:34 and 35 and or PRM2 using primers of SEQ ID No: 36 and 37.

8. The method of claim 1, further comprising determining if the biological sample is seminal fluid, comprising the step of detecting RNA associated with KLK2 using primers of SEQ ID No:38 and 39, and/or MSMB using primers of SEQ ID No:40 and 41 and/or TGM4 using primers of SEQ ID No: 42 and 43.

9. The method of claim 1, further comprising determining if the biological sample is menstrual fluid, comprising the step of detecting RNA associated with MMP10 using primers of SEQ ID No:44 and 45, and/or STC1 using primers of SEQ ID No:46 and 47 and/or MMP3 using primers of SEQ ID No:48 and 49 and/or MMP11 using primers of SEQ ID No. 50 and 51.

10. The method of claim 1, further comprising determining if the biological sample is vaginal material, comprising the step of detecting RNA associated with CYP2B7P using primers of SEQ ID No:52 and 53 and/or L.gass using primers of SEQ ID No: 54 and 55 and/or L.crisp of SEQ ID No: 56 and 57.

11. The method of claim 1, further comprising testing for the presence of RNA of all of HBD, SLC4A1, GYPA, FDCSP, HTN3, STATH, PRM1, TNP1, PRM2, KLK2, MSMB, TGM4, MMP10, STC1, MMP3, MMP11, CYP2B7P, Lactobacillus gasseri (L.gass) and Lactobacillus crispatus (L.crisp) in the biological sample.

12. The method of claim 1, further comprising detecting the presence of RNA of any one or more of HTN3 and FDCSP; and/or SLC4A1, HBD, STC1 and MMP10 and/or TNP1, PRM1, KLK2, MSMB and CYP2B79.

13. The method of claim 3, wherein the primers are labelled.

14. The method of claim 13, wherein the primers are labelled with a fluorescence label, biotin, radioactive or non-radioactive label.

15. The method of claim 1, wherein the RNA is detected using an amplification method.

16. The method of claim 15, wherein the amplification method is selected from the group comprising polymerase chain reaction (PCR), reverse transcriptase PCR (RT-PCR), quantitative reverse transcriptase PCR (qRT-PCR), multiplex PCR, multiplex ligation-dependent probe amplification (MLPA) or quantitative PCR (Q-PCR).

17. A kit for use in the method of claim 1, the kit comprising at least one primer pair selected from SEQ ID Nos: 20 and 21, 22 and 23, 24 and 25, 26 and 27, 28 and 29, 30 and 31, 32 and 33, 34 and 35, 36 and 37, 38 and 39, 40 and 41, 42 and 43, 44 and 45, 46 and 47, 48 and 49, 50 and 51, 52 and 53, 54 and 55, and 56 and 57.
